SEQUENCE LISTING (84 sequences)

SEQ ID NO: 1 - protein, 135 aa, Rhizobium sp.
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 2 - protein, 135 aa, synthetic construct
  REGION 1..135: note = Rhavi-A5T
FDASNFKDFSSIASASSSWQNQSGSTMIIQVDSFGAVSGQYVNRAQGTGCQNSPYPLTGRVNGAFIAFSVGWNAATENCNSATGWTGYAQVNGNATEIVTSWNLAYEGGSGPAIEQGQDTFQYVPTTENKSLLKD

SEQ ID NO: 3 - protein, 185 aa, synthetic construct
  REGION 1..185: note = SBD1 (silac acid binding domain 1)
VIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 4 - protein, 192 aa, unidentified
  REGION 1..192: note = SBD2 NanL (81-272)
IPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 5 - protein, 191 aa, Streptococcus pneumoniae
  REGION 1..191: note = SBD3 NanB (40-230)
SPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 6 - protein, 189 aa, Streptococcus pneumoniae
  REGION 1..189: note = SBD4 NanC 82-270
EETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 7 - protein, 381 aa, Salmonella sp.
  REGION 1..381: note = NanH Salmonella
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 8 - protein, 404 aa, Salmonella sp.
  REGION 1..404: note = NanH2
MKKFIKILKVLSMAIVLSACNINGIFASNLNTTNEPQKTTVFNKNDNTWNAQYFRIPSLQTLADGTMLAFSDIRYNGAEDHAYIDIGAAKSTDNGQTWDYKTVMENDRIDSTFSRVMDSTTVVTDTGRIILIAGSWNKNGNWASSTTSLRSDWSVQMVYSDDNGETWSDKVDLTTNKARIKNQPSNTIGWLAGVGSGIVMSDGTIVMPIQIALRENNANNYYSSVIYSKDNGETWTMGNKVPDPKTSENMVIELDGALIMSSRNDGKNYRASYISYDMGSTWEVYDPLHNKISTGNGSGCQGSFIKVTAKDGHRLGFISAPKNTKGGYVRDNITVYMIDFDDLSKGIRELCSPYPEDGNSSGGGYSCLSFNDGKLSILYEANGNIEYKDLTDYYLSIENNKKLK

SEQ ID NO: 9 - protein, 396 aa, unidentified
  REGION 1..396: note = NanH3 Trypanosoma cruzi 4-399
GSSRVELFKRQSSKVPFEKDGKVTERVVHSFRLPALVNVDGVMVAIADARYETSFDNSLIDTVAKYSVDDGETWETQIAIKNSRASSVSRVVDPTVIVKGNKLYVLVGSYNSSRSYWTSHGDARDWDILLAVGEVTKSTAGGKITASIKWGSPVSLKEFFPAEMEGMHTNQFLGGAGVAIVASNGNLVYPVQVTNKKKQVFSKIFYSEDEGKTWKFGKGRSAFGCSEPVALEWEGKLIINTRVDYRRRLVYESSDMGNTWLEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPA

SEQ ID NO: 10 - protein, 1035 aa, Streptococcus pneumoniae
  REGION 1..1035: note = NanA sialidase amino acid sequence
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 11 - protein, 781 aa, Vibrio cholerae
  REGION 1..781: note = NanH sialidase amino acid sequence
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 12 - protein, 192 aa, Vibrio cholerae
  REGION 1..192: note = SBD of Vibrio cholerae NanH sialidase (aa 25-216)
ALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 13 - protein, 328 aa, synthetic construct
  REGION 1..328: note = Rhavi-SBD1
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 14 - protein, 335 aa, synthetic construct
  REGION 1..335: note = Rhavi-SBD2
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 15 - protein, 334 aa, synthetic construct
  REGION 1..334: note = Rhavi-SBD3
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 16 - protein, 332 aa, synthetic construct
  REGION 1..332: note = Rhavi-SBD4
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 17 - protein, 328 aa, synthetic construct
  REGION 1..328: note = SBD1-Rhavi
MVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 18 - protein, 335 aa, synthetic construct
  REGION 1..335: note = SBD2-Rhavi
MIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATGGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 19 - protein, 334 aa, synthetic construct
  REGION 1..334: note = SBD3-Rhavi
MSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 20 - protein, 332 aa, synthetic construct
  REGION 1..332: note = SBD4-Rhavi
MEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 21 - protein, 175 aa, unidentified
  REGION 1..175: note = RIB
AEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPS

SEQ ID NO: 22 - protein, 405 aa, unidentified
  REGION 1..405: note = Sip
DTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 23 - protein, 914 aa, synthetic construct
  REGION 1..914: note = Rhavi-Rib-Sip-SBD1
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 24 - protein, 914 aa, synthetic construct
  REGION 1..914: note = Rhavi-Sip-Rib-SBD1
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDTDPNSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 25 - protein, 922 aa, synthetic construct
  REGION 1..922: note = Rib-Sip-Rhavi-SBD1
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 26 - protein, 918 aa, synthetic construct
  REGION 1..918: note = Sip-Rib-Rhavi-SBD1
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 27 - protein, 921 aa, synthetic construct
  REGION 1..921: note = Rhavi-Rib-Sip-SBD2
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 28 - protein, 921 aa, synthetic construct
  REGION 1..921: note = Rhavi-Sip-Rib-SBD2
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 29 - protein, 920 aa, synthetic construct
  REGION 1..920: note = Rhavi-Rib-Sip-SBD3
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 30 - protein, 920 aa, synthetic construct
  REGION 1..920: note = Rhavi-Sip-Rib-SBD3
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 31 - protein, 918 aa, synthetic construct
  REGION 1..918: note = Rhavi-Rib-Sip-SBD3
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 32 - protein, 918 aa, synthetic construct
  REGION 1..918: note = Rhavi-Sip-Rib-SBD3
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 33 - protein, 929 aa, synthetic construct
  REGION 1..929: note = Rib-Sip-Rhavi-SBD2
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 34 - protein, 925 aa, synthetic construct
  REGION 1..925: note = Sip-Rib-Rhavi-SBD2
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 35 - protein, 928 aa, synthetic construct
  REGION 1..928: note = Rib-Sip-Rhavi-SBD3
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 36 - protein, 924 aa, synthetic construct
  REGION 1..924: note = Sip-Rib-Rhavi-SBD2
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSSPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 37 - protein, 7 aa, synthetic construct
GGGGSSS

SEQ ID NO: 38 - skipped

SEQ ID NO: 39 - protein, 5 aa, synthetic construct
  REGION 1..>5: note = The entire sequence of amino acids 1-5 can be repeated one or more times.
GGGGS

SEQ ID NO: 40 - protein, 6 aa, synthetic construct
GGGGGG

SEQ ID NO: 41 - protein, 15 aa, synthetic construct
GGGGSGGGGSGGGGS

SEQ ID NO: 42 - protein, 30 aa, synthetic construct
GGGGSGGGGSGGGGSGGGGSGGGGSGGGGS

SEQ ID NO: 43 - protein, 18 aa, synthetic construct
KESGSVSSEQLAQFRSLD

SEQ ID NO: 44 - protein, 14 aa, synthetic construct
EGKSSGSGSESKST

SEQ ID NO: 45 - skipped

SEQ ID NO: 46 - protein, 8 aa, synthetic construct
GGGGGGGG

SEQ ID NO: 47 - protein, 12 aa, synthetic construct
GSAGSAAGSGEF

SEQ ID NO: 48 - protein, 5 aa, synthetic construct
  REGION 1..>5: note = The entire sequence of amino acids 1-5 can be repeated one or more times.
EAAAK

SEQ ID NO: 49 - protein, 7 aa, synthetic construct
  REGION 2..>6: note = The entire sequence of amino acids 2-6 can be repeated one or more times.
AEAAAKA

SEQ ID NO: 50 - protein, 46 aa, synthetic construct
AEAAAKEAAAKEAAAKEAAAKALEAEAAAKEAAAKEAAAKEAAAKA

SEQ ID NO: 51 - protein, 7 aa, synthetic construct
  REGION 2..>6: note = The entire sequence of amino acids 2-6 can be repeated one or more times.
  REGION 1..>7: note = The entire sequence of amino acids 1-7 can be repeated one or more times.
AEAAAKA

SEQ ID NO: 52 - protein, 12 aa, synthetic construct
AEAAAKEAAAKA

SEQ ID NO: 53-56 - skipped (4 entries)

SEQ ID NO: 57 - protein, 14 aa, synthetic construct
APAPAPAPAPAPAP

SEQ ID NO: 58 - protein, 21 aa, synthetic construct
GAPGGGGGAAAAAGGGGGGAP

SEQ ID NO: 59 - protein, 39 aa, synthetic construct
GAPGGGGGAAAAAGGGGGGAPGGGGGAAAAAGGGGGGAP

SEQ ID NO: 60 - protein, 57 aa, synthetic construct
GAPGGGGGAAAAAGGGGGGAPGGGGGAAAAAGGGGGGAPGGGGGAAAAAGGGGGGAP

SEQ ID NO: 61 - skipped

SEQ ID NO: 62 - protein, 5 aa, synthetic construct
GGGGS

SEQ ID NO: 63 - protein, 5 aa, synthetic construct
EAAAK

SEQ ID NO: 64 - skipped

SEQ ID NO: 65 - protein, 6 aa, synthetic construct
HHHHHH

SEQ ID NO: 66 - protein, 10 aa, synthetic construct
MSYYHHHHHH

SEQ ID NO: 67-69 - skipped (3 entries)

SEQ ID NO: 70 - protein, 934 aa, synthetic construct
  REGION 1..934: note = Rib-Sip-Rhavi-SBD4
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNLEHHHHHH

SEQ ID NO: 71 - protein, 930 aa, synthetic construct
  REGION 1..930: note = Sip-Rib-Rhavi-SBD4
MDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNAAAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSGGGGSSSFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTNLEHHHHHH

SEQ ID NO: 72 - protein, 317 aa, synthetic construct
  REGION 1..317: note = Rhavi-Rib
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIP

SEQ ID NO: 73 - protein, 548 aa, synthetic construct
  REGION 1..548: note = Rhavi-Sip
MFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKDGGGGSSSDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVFVADQKVSLNTISEGMTPEAATTIVSPMKTYSSAPALKSKEVLAQEQAVSQVAANEQVSPAPVKSITSEVPAAKEEVKPTQTSVSQLTTVSPASVAAETPAPVAKVAPVRTVAAPRVASAKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFN

SEQ ID NO: 74 - protein, 364 aa, synthetic construct
  REGION 1..364: note = Rib-SBD1
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 75 - protein, 371 aa, synthetic construct
  REGION 1..371: note = Rib-SBD3
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAIPEGILMEKNNVDIAEGQGYSLDQEAGAKYVKAMTQGTIILSYKSTSENGIQSLFSVGNSTAGNQDRHFHIYITNSGGIGIELRNTDGVFNYTLDRPASVRALYKGERVFNTVALKADAANKQCRLFANGELLATLDKDAFKFISDITGVDNVTLGGTKRQGKIAYPFGGTIGDIKVYSNALSDEELIQATG

SEQ ID NO: 76 - protein, 370 aa, synthetic construct
  REGION 1..370: note = Rib-SBD3
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAASPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQ

SEQ ID NO: 77 - protein, 368 aa, synthetic construct
  REGION 1..368: note = Rib-SBD4
MAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPSAAAEETPVLEKNNVTLTGGGENVTKELKDKFTSGDFTVVIKYNQSSEKGLQALFGISNSKPGQQNSYVDVFLRDNGELGMEARDTSSNKNNLVSRPASVWGKYKQEAVTNTVAVVADSVKKTYSLYANGTKVVEKKVDNFLNIKDIKGIDYYMLGGVKRAGKTAFGFNGTLENIKFFNSALDEETVKKMTTN

SEQ ID NO: 78 - DNA, 405 bp, Rhizobium sp.
  source 1..405: mol_type = unassigned DNA
tttgatgcgagtaactttaaagatttcagctctattgcgagcgccagtagctcttggcagaatcagagtggcagcaccatgattatccaggtggattctttcggcaacgttagtggccagtatgttaatcgtgcgcagggcacgggttgccagaactctccgtacccgctgaccggccgcgtgaatggcacgtttatcgccttcagcgttggctggaacaattctaccgaaaactgtaatagtgcaaccggctggacgggttatgcgcaggtgaacggtaacaataccgaaattgttacgagctggaatctggcctatgaaggcggttctggcccggcaatcgaacagggtcaggatacctttcagtacgttccgaccacggaaaacaaaagcctgctgaaagat

SEQ ID NO: 79 - skipped

SEQ ID NO: 80 - DNA, 555 bp, unidentified
  source 1..555: mol_type = unassigned DNA
gtgatagaaaaagaagatgttgaaaccaatgcttcaaatggtcagagagttgatttatcaagtgaactagataaactaaagaaacttgaaaacgcaacagttcacatggagtttaagccagatgccaaggccccagcattctataatctcttttctgtgtcaagtgctactaaaaaagatgagtacttcactatggcagtttacaataatactgctactctagaggggcgtggttcggatgggaaacagttttacaataattacaacgatgcacccttaaaagttaaaccaggtcagtggaattctgtgactttcacagttgaaaaaccgacagcagaactacctaaaggccgagtgcgcctctacgtaaacggggtattatctcgaacaagtctgagatctggcaatttcattaaagatatgccagatgtaacgcatgtgcaaatcggagcaaccaagcgtgccaacaatacggtttgggggtcaaatctacagattcggaatctcactgtgtataatcgtgctttaacaccagaagaggtacaaaaacgtagt

SEQ ID NO: 81 - DNA, 576 bp, unidentified
  source 1..576: mol_type = unassigned DNA
attccggaaggcattctgatggaaaagaacaatgttgatatagccgaagggcagggatacagcctggaccaggaggctggcgccaaatatgtaaaggctatgacccagggtacaattatcctatcctataaatcaacaagtgaaaatggaatccagtctctgttcagcgtaggaaacagtaccgcgggcaatcaggacaggcatttccacatctatataacaaactctggcggcataggcatagaacttagaaacacggacggtgtcttcaattatacattggacagaccggcatcagtgcgtgctctgtataaaggagaacgggtattcaatacagttgcattaaaagccgatgcagcaaataaacagtgcagattatttgcaaatggcgagcttctcgcaacactggataaagatgcgtttaagtttatcagtgacattacaggcgtagataacgttacattaggcggcacgaagaggcaggggaagattgcatatccgttcgggggaacgattggtgacattaaggtatatagtaatgcactgtccgacgaagaattgatacaggcaacagga

SEQ ID NO: 82 - DNA, 576 bp, unidentified
  source 1..576: mol_type = unassigned DNA
attcccgaaggcatcctgatggagaaaaataacgttgatattgccgagggccagggctattctttggatcaagaggccggtgccaagtatgtaaaagcaatgacacaagggaccattattctgtcatacaaatcgacctcagagaatggcatccaatcgttattctcagtcgggaatagtactgctggtaatcaggaccgtcattttcatatctatattacaaactcggggggcattggaatcgagcttcgtaacacagatggcgtattcaactatactttggaccgcccggcgtctgttcgcgcgctgtataaaggggagcgcgtgttcaatacggttgctttgaaggcggacgccgccaataagcagtgtcgcttgttcgcaaatggcgaattgttagctacgcttgacaaggacgcatttaagtttatttccgacatcactggggtggacaatgtaacgttgggtggaaccaaacgccagggcaaaattgcatacccgttcggagggacaatcggcgacattaaagtctatagcaacgcgctgagtgacgaagagcttatccaggctaccgga

SEQ ID NO: 83 - DNA, 573 bp, unidentified
  source 1..573: mol_type = unassigned DNA
tctcctatttttcaaggaggttcatatcaactgaacaataagagtatagatatcagctctttgttattagataaattgtctggagagagtcagacagtagtaatgaaatttaaagcagataaaccaaactctcttcaagctttgtttggcctatctaatagtaaagcaggctttaaaaataattacttttcaattttcatgagagattctggtgagataggtgtagaaataagagacgcccaaaagggaataaattatttattttccagaccagcttcattatggggaaaacataaaggacaggcagttgaaaatacactagtatttgtatctgattctaaagataaaacatacacaatgtatgttaatggaatagaagtgttctctgaaacagttgatacatttttgccaatttcaaatataaatggtatagataaggcaacactaggagctgttaatcgtgaaggtaaggaacattacctcgcaaaaggaagtattgatgaaatcagtctatttaacaaagcaattagtgatcaggaagtttcaactattcccttgtcaaatccatttcag

SEQ ID NO: 84 - DNA, 567 bp, unidentified
  source 1..567: mol_type = unassigned DNA
gaagaaactcctgtcttagaaaaaaataatgttactttaacagggggcggagaaaatgttactaaagagttaaaggataaatttactagcggtgactttactgtagtgattaagtacaatcagtcaagtgagaaaggcttacaagctctgtttggaatatctaattccaaacccggtcaacaaaatagttatgtagatgtgttccttagagacaatggtgagttggggatggaagcgcgtgatacttcttccaataaaaataacctagtatccagacctgcttcagtttggggtaagtacaaacaagaggctgtgactaacactgttgcagtagtagcagattcagtcaaaaaaacatattctttatacgcaaatggtacaaaagtagtagaaaagaaagtggataatttcctaaacatcaaggatattaaaggtattgattactatatgcttgggggagtgaaacgtgcaggaaaaacggcgtttggttttaacggaacactagaaaatatcaaattctttaatagtgcattggatgaagaaactgttaaaaagatgacaacaaac

(19) United States
(12) Patent Application Publication
(10) Pub. No.: US 2026/0115270 A1
(43) Pub. Date: Apr. 30, 2026

(54) MULTIPLE ANTIGEN PRESENTING SYSTEM (MAPS) CROSS-LINKED USING A BIFUNCTIONAL FUSION PROTEIN AND ITS USE IN VACCINES PROTEIN AND ITS USE IN VACCINES

(71) Applicant: THE CHILDREN'S MEDICAL CENTER CORPORATION, Boston, MA (US)

(72) Inventors: Richard MALLEY, Beverly, MA (US); Fan ZHANG, West Roxbury, MA (US); Yingjie LU, West Roxbury, MA (US)

(73) Assignee: THE CHILDREN'S MEDICAL CENTER CORPORATION, Boston, MA (US)

(21) Appl. No.: 19/120,643

(22) PCT Filed: Oct. 13, 2023
(86) PCT No.: PCT/US2023/076909

§ 371 Date: Apr. 11, 2025

Related U.S. Application Data
(60) Provisional application No. 63/416,482, filed on Oct. 14, 2022.

(51) Int. Cl. A61K 39/09; A61K 39/00; A61K 39/116; A61P 37/04; C07K 14/195

(52) U.S. Cl. CPC A61K 39/092; A61K 39/116; A61P 37/04; C07K 14/195; A61K 2039/6031; A61K 2039/70; C07K 2319/00

(57) ABSTRACT

Technologies for the prevention and/or treatment of pathogenic or microbial infections. Compositions relate to compositions, vaccines and methods comprising an immunogenic multiple antigen presenting system (MAPS), where two or more biotinylated polysaccharide antigens are interconnected or joined together by one or more fusion proteins comprising (i) a sialic acid binding domain (SBD) and (ii) a biotin-binding moiety (BBM), thereby facilitating the joining multiple polysaccharide antigens together in the complex to form a cross-linked MAPS-X immunogenic complex. The polysaccharide antigens that are linked can be on the same polysaccharide macromolecule or on distinct polysaccharide macromolecules.

### CROSS-REFERENCED TO RELATED APPLICATIONS

This application is a 371 National Phase Entry of International Patent Application which claims benefit under 35 U.S.C. § 119(e) of U.S. Provisional Application No. 63/416,482 filed Oct. 14, 2022, the contents of which is incorporated herein by reference in its entirety.

### FIELD OF THE INVENTION

The present invention relates to technologies, compositions, and methods for the prevention and/or treatment of bacterial infections.

### SEQUENCE LISTING

The instant application contains a Sequence Listing which was created on Oct. 12, 2023 with the filename “701039-000103WOPT_SL.xml” and has a file size of 111, 716 bytes.

### BACKGROUND OF INVENTION

Sialic acid is a monosaccharide that is widely present in animals and, to a lesser extent, in microorganisms, including fungi, bacteria and viruses. Sialic acid modification is commonly found in glycoproteins and glycolipids in mammalian cells and is important for cell function. However, certain bacteria and virus can incorporate sialic acid onto their surface capsule, such as polysaccharides in the case of bacteria or glycoproteins in the case of virus, so that they can evade host immune defense during infection by using sialic acid (a self-antigen for the host) as a shield. For the same reason, it is difficult to generate immune responses to sialic acid-containing bacterial capsular polysaccharides or viral glycoproteins when used as vaccine targets. In most cases, such sialic acid modification is part of the important epitopes to which antibodies are most effective in functionally removing the target bacterial or viral pathogens.

Thus, there is a need to develop a method to overcome the “immune suppression” caused by sialic acid modification in bacterial or viral components and induce robust immune responses to these molecules during vaccination.

### SUMMARY OF THE INVENTION

The technology disclosed herein relates to a multiple antigen presenting system (MAPS) immunogenic complex that comprises a bifunctional fusion protein intermediate that cross-links antigenic polysaccharides in the complex.

Herein, the technology relates to a cross-linked MAPS immunogenic complex, herein referred to a “MAPS-X immunogenic complex”, which comprises a bifunctional fusion protein which non-covalently associates with two antigenic polysaccharides (referred to herein as PS1 and PS2) in the complex, therefore forming a large immunogenic complex where 2 or more polysaccharides (e.g., PS1, PS2. PS3, PS4, PS5, PS6 etc.) are connected via the bifunctional fusion protein. In all aspects of the technology disclosed herein, a bifunctional fusion protein that connects the two polysaccharide antigens comprises at least (i) a biotin binding moiety (BBM) and (ii) a sialic acid binding domain (SBD) as disclosed herein, and is referred to herein as a “SBD-BBM fusion” protein. Additionally, an added advantage of the presence to the bifunctional SBD-BBM fusion protein is that the sialic acid-binding domain (SBD) protein can reduce or overcome the immune suppression of native sialic acid on the surface of bacterial or other pathogenic polysaccharides.

Accordingly, herein the inventors have developed a MAPS-X immunogenic complex where two or more biotinylated antigenic polysaccharides are interconnected or joined together by one or more SBD-BBM fusion proteins. As the number of biotinylated polysaccharide antigens and number of SBD-BBM fusion proteins increases, it increases the network or arrangement of connected polysaccharides via the bifunctional SBD-BBM fusion protein. As such, the MAPS-X immunogenic complex as disclosed herein is modified from, and an improvement of the Multiple Antigen Presenting System (MAPS) as previously disclosed in U.S. Pat. No. 10,766,932, which is incorporated herein in its entirety by reference, which discloses a fusion protein comprising a biotin-binding protein and antigen which can non-covalently associate to only one polysaccharide at a time, therefore allowing multiple protein antigens to be associated with one polysaccharide antigen, but in contrast to the current invention, not facilitating the joining multiple polysaccharides together in the complex. Accordingly, and without wishing to be bound by theory, the disclosed MAPS-X system provides advantages over existing MAPS complexes, such advantages include but not are limited to, (i) generation of a larger size of a MAPS-X immunogenic complex based on the joining of multiple polysaccharides, where a larger sized complexes are advantageous for generating an immune response in a subject to the polysaccharides and/or any protein antigen present on the immune complex, and (ii) suppressing the immune suppression by sialic acid molecules on the surface of bacterial polysaccharides.

In some embodiments, and as an added benefit, the non-covalent association of the SBD to a sialic acid on the antigenic polysaccharide blocks the immune suppressive function of sialic acids on an antigenic polysaccharide. That is, without being limited to theory, a sialic acid-binding domain (SBD) can bind to a sialic acid present on the surface of a polysaccharide and essentially mask (e.g., shield) the immune tolerance of the antigenic polysaccharide. Stated differently, the binding of the SBD-BBM of the fusion protein to sialic acid on a polysaccharide reduces the hosts' exposure to the sialic acids, which are normally recognized as a self-antigen by the host. With reduced exposure due to the masked sialic acid molecules, the host recognizes the polysaccharide as foreign and thus the binding of SBD of the SBD-BBM fusion protein on the polysaccharide increases the immunogenicity of the polysaccharide to the host. Herein the inventors have demonstrated that a SBD-BBM fusion protein in a MAPS-X complex as disclosed herein can quench the ability of sialic acids on the surface of capsular polysaccharides to evade a host immune system, and therefore improve the host's immune response to the polysaccharide when administered to the subject. That is, the technology disclosed herein relates to the use of a fusion protein comprising a sialic acid-binding domain (SBD) and BBM to overcome the immune suppression of native sialic acid on the surface of a polysaccharide, e.g., a polysaccharide with sialic acids on the surface, such as but not limited to GBS polysaccharides. Surprisingly, the inventors have also discovered that use of a fusion protein comprising a sialic acid-binding domain (SBD) can also increase the immunogenicity of a plurality of different polysaccharide, even if the polysaccharide does not comprise sialic acid molecules on it surface.

Aspects of the technology described herein relate to methods, compositions and vaccines comprising a MAPS-X immunogenic complex comprising a bi-functional fusion protein which comprises a sialic acid-binding domain (SBD) and a biotin-binding moiety (BBM), referred to herein as SBD-BBM fusion protein, where the fusion protein non-covalently associates to at least first antigenic biotinylated polysaccharide via the BBM of the fusion protein, and at least a second antigenic polysaccharide via the SBD of the fusion protein, therefore indirectly joining the first and second polysaccharide antigens. In some embodiments, both the first and second polysaccharide antigens are biotinylated. In some embodiments, the first or second polysaccharide antigen, or both, comprises a sialic acid moiety. Thus, the SBD-BBM serves as a cross-linking moiety to link two polysaccharide antigens together (see, e.g., FIGS. 1B and 1E).

The MAPS-X complex as disclosed herein comprises biotinylated polysaccharides and a bifunctional SBD-BBM fusion protein. In brief, a bifunctional SBD-BBM serves as both a carrier protein and a linking protein, which can link two polysaccharide chains together, thereby forming a cross-linked MAPS-X complex. Stated differently, a MAPS-X complex comprises at least two polysaccharide antigens, which can be (i) on the same polysaccharide macromolecule or (ii) on different polysaccharide macromolecules, where the two polysaccharide antigens are linked together via the SBD-BBM fusion protein which non-covalently associates with each of the two polysaccharide antigens. As such, the SBD-BBM fusion protein serves as a linking protein that is also functions as a carrier protein to link two polypeptide antigens.

In some embodiments, if the two polypeptide antigens, referred herein and throughout the specification as PS1 and PS2 respectively, are located on the same macromolecule, e.g., on the same polymer chain, or on different branches of a polymer of the same macromolecule, the SBD-BBM fusion protein serves an intra-macromolecule linkage role to form a MAPS-X immunogenic complex. In alternative embodiments, if the two polypeptide antigens are located on a different macromolecule, e.g., one PS1 is from a distinct polysaccharide macromolecule, and the other PS2 is a distinct polysaccharide macromolecule (e.g., from a different pathogen and/or a specific serotype from the macromolecule used in P1), the SBD-BBM fusion protein serves an inter-macromolecule linkage role, to form a MAPS-X immunogenic complex with more than one polysaccharide macromolecule.

Accordingly, term “polysaccharide antigen” as used herein refers to a region or portion of a polysaccharide macromolecule that comprises biotin and/or sialic acid molecules. Thus, two “polysaccharide antigens” can be present either (i) on the same polysaccharide molecule (e.g., the same macromolecule of PS polymer chain, including any branches), thereby forming links within the same polysaccharide polymer macromolecule, or alternatively, (ii) each “polysaccharide antigen” can be present in two distinct polysaccharide polymer macromolecules, thereby enabling the joining different macromolecules. As such, in some embodiments, a MAPS-X complex can comprise intra-linkage of polysaccharide antigens within the same polysaccharide polymer macromolecule, and/or inter-linkage of polysaccharide antigens from two or more distinct polysaccharide polymer macromolecules.

In some embodiments, the SBD-BBM fusion protein does not comprise a polypeptide antigen. In alternative embodiments, the SBD-BBM fusion protein can further comprise at least one polypeptide antigen. Accordingly, aspects of the technology disclosed herein also relates to a bi-functional fusion protein comprising, in any order, (i) a sialic acid-binding domain (SBD), (ii) a biotin-binding moiety (BBM), and (iii) at least 1, or at least 2, or at least 3, at least 4, at least 5, or more than 5 polypeptide antigens, and is broadly referred to herein as SBD-[Ag]n-BBM fusion protein, where n is the number of antigenic polypeptide located between the SBD and BBM. It is envisioned that the antigen in the SBD-[Ag]n-BBM fusion protein can be located at the N-terminal, or the C-terminal of the fusion protein, or between the SBD and the BBM. For example, exemplary fusion proteins are disclosed in Table 1 and 2 herein.

Disclosed herein is data demonstrating use of a bi-functional SBD-BBM fusion protein fusion where the SBD moiety can non-covalently bind to a sialic acid on a polysaccharide antigen and blocks its exposure to (and suppression on) antigen-presenting cells until the antigens are internalized and processed for proper epitope-presentation, while the BBM can non-covalently bind to a biotinylated polysaccharide, or other biotinylated scaffold, ligands, or other molecules. The technology disclosed herein also describes the use of sialic acid-binding domain to overcome immune suppression caused by sialic acid modification in bacterial or viral components and induce robust immune responses to both the antigenic polysaccharides and any polypeptide antigens in a MAPS-X immunogenic complexes comprising bifunctional SBD-BBM fusion proteins during vaccination.

In some embodiments as disclosed herein, SBD-BBM fusion protein fusion is useful for joining antigenic polysaccharides that do not comprise sialic acid on their surfaces. For example, in some embodiments, the SBD-BBM fusion protein can non-covalently associate with biotin on a first biotinylated polysaccharide, and the SBD can form non-covalent associate with a second biotinylated polysaccharide, even if the second biotinylated polysaccharide does not comprise sialic acid moieties. Without wishing to be bound by theory, such an interaction is due to the association of the SBD with a positively charged second biotinylated polysaccharide.

In some embodiments, a bifunctional SBD-BBM fusion protein useful in the MAPS-X immunogenic complex as disclosed herein comprises, in any order (i) at least one SBD selected from any of SBD1, SBD2, SBD3, SBD4, NanH, NanH2, NanH3, VcNanH, or having an amino acid sequence with at least 80%, or at least 85%, or at least 90%, or at least 95% sequence identity to any of: SEQ ID NOs: 2 or 112-120, and a BBM. In some embodiments, a bifunctional SBD-BBM fusion protein useful in the MAPS-X immunogenic complex as disclosed herein comprises, in any order (i) at least SBD having an amino acid sequence with at least 80%, or at least 85%, or at least 90%, or at least 95% sequence identity to SEQ ID NO: 2 (SBD1), or at least 80%, or at least 85%, or at least 90%, or at least 95% sequence identity to SEQ ID NO: 112 (SBD2), and (ii) a BBM which is Rhizavidin having an amino acid sequence with at least 80%, or at least 85%, or at least 90%, or at least 95% sequence identity to SEQ ID NO: 1 or SEQ ID NO: 8.

In all aspects of the bifunctional SBD-BBM fusion protein disclosed herein, BBM is a rhizavidin polypeptide having an amino acid sequence comprising SEQ ID NO: 1 or 2, or an amino acid sequence having at least 80%, or at least 85%, or at least 90%, or at least 95% sequence identity to SEQ ID NO: 1 or SEQ ID NO: 2.

For illustrative purposes only, referring to FIG. 1B, another aspect of the present invention relates to methods, compositions and vaccines comprising a MAPS-X immunogenic complex, wherein a composition at least one species of MAPS-X immunogenic complex, wherein each species of the MAPS-X immunogenic complex comprises; (i) at least a first biotinylated polysaccharide antigen (PS1) comprising at least one biotin molecule, (ii) at least a second polysaccharide antigen (PS2) comprising at least one sialic acid molecule, and (iii) at least one SBD-BBM bifunctional fusion protein as disclosed herein, each fusion protein comprises at least a biotin-biotin moiety (BBM) and a sialic acid binding domain (SBD). When the BBM of at least one SBD-BBM fusion protein non-covalently associates with at least PS1 and/or a SBD non-covalently associates with at least one sialic acid on PS2, this results in PS1 and PS2 being indirectly linked (or joined) via the non-covalent association with the SBD-BBM fusion proteins(s), which serves as a cross-linking intermediate protein to form a MAPS-X immunogenic complex.

Where the first (PS1) and second polysaccharide antigen (PS2) are located on the same macromolecule, e.g., on the same polymer chain, or on different branches of a polymer of the same macromolecule, the SBD-BBM fusion protein serves an intra-macromolecule linkage role to form a MAPS-X immunogenic complex. Where the PS1 and PS2 are located on a different macromolecule, e.g., PS1 is located on a specific polysaccharide macromolecule, and the other PS2 is located a distinct polysaccharide macromolecule (e.g., from a different pathogen and/or a specific serotype to the PS1 macromolecule), the SBD-BBM fusion protein serves an inter-macromolecule linkage role, to form a MAPS-X immunogenic complex with more than one polysaccharide macromolecule.

Optionally, where the MAPS-X complex comprises more than one bifunctional SBD-BBM fusion proteins, e.g., a 2^{nd} a bifunctional SBD-BBM fusion protein (FP2), a SBD of at least a second fusion protein (FP2) non-covalently associates with at least one sialic acid molecule on the first biotinylated polysaccharide antigen (PS1) and the BBM of this second fusion protein non-covalently associates with at least biotin molecule on the second biotinylated polysaccharide antigen (PS2), therefore further joining the PS1 and PS2 via the association with the SBD-BBM fusion proteins(s) serving as a cross-linking intermediate.

For illustrative purposes only, referring to FIG. 1E shows an exemplary embodiment of MAPS-X immunogenic complex species, which forms a larger complex as compared to a MAPS complex that does not comprise a bifunctional SBD-BBM fusion protein. For illustrative purposes only, shown is a MAPS-X complex of 5 polysaccharide antigens (PS1-PS5), a first SBD-BBM fusion protein (FP1) can associate with PS1 and PS2, and a second SBD-BBM fusion protein (FP2) can associate with PS2 and PS3, and a third SBD-BBM fusion protein (FP3) can associate with, e.g., PS3 and PS4, and a fourth SBD-BBM fusion protein (FP4) (FP2) can associate with, e.g., PS4 and PS5, etc. thereby forming a MAPS-X immunogenic complex, where the polysaccharide antigens are joined or cross-linked via the SBD-BBM fusion protein intermediate. Some SBD-BBM fusion proteins can also associate with polysaccharides already existing in the complex, for example, a fifth SBD-BBM fusion protein (FP5) can associate with PS5 and PS1, a sixth SBD-BBM fusion protein (FP6) can associate with PS5 and PS3, strengthening the MAPS-X complex. The polysaccharide antigens, e.g., PS1-PS5 can be from the same subtype (or serotype) of bacteria or pathogen. In alternative embodiments, each polysaccharide antigen (e.g., PS1, PS2, PS3, PS4, PS5, PS6 etc.) can be from a different subtypes of bacteria or pathogen. Accordingly, due to the presence of a plurality of bifunctional SBD-BBM fusion proteins which can non-covalently associate with two polysaccharide antigens, a MAPS-X immunogenic complex as disclosed herein can cross-link a plurality of polysaccharide antigens in the complex, therefore forming larger complexes than prior MAPS complexes which comprise biotin-binding fusion proteins that can non-covalently associate with only one polysaccharide at a time.

In some embodiments, the MAPS-X immunogenic complex can comprise the following non-covalent associations; PS1-(SBD-BBM fusion protein)-PS2, where the BBM of the SBD-BBM fusion protein non-covalently associates with a biotin on the first polysaccharide antigen (PS1), and the SBD of the SBD-BBM fusion protein non-covalently associates with a sialic acid on the second polysaccharide antigen (PS2), to form a cross-linked MAPS-X immunogenic complex. In some embodiments, the MAPS-X immunogenic complex can comprise the following non-covalent associations: PS1-(SBD-BBM fusion protein)-PS2, where the SBD of the SBD-BBM fusion protein non-covalently associates with a sialic acid on the first polysaccharide antigen (PS1), and the BBM of the SBD-BBM fusion protein non-covalently associates with a biotin on the second polysaccharide antigen (PS2), to form a MAPS-X immunogenic complex. As either the first (PS1), or second antigenic polysaccharides antigen (PS2), or both, can have both biotin molecules and sialic acid molecules on their surface, the immune complex can comprise both non-covalent associations: PS1-(SBD-BBM fusion protein)-PS2 and PS1-(SBD-BBM fusion protein)-PS2 (see, e.g., FIG. 1B). It is also envisioned that the SBD-BBM fusion protein further comprises at least one, or at least 2, or at least 3 or more antigenic polypeptide antigens, and can optionally be located between a BBM and a SBD polypeptide, or in any order.

For exemplary purposes only, the inventors demonstrate herein in the Examples the generation of a MAPS-X immunogenic complex comprising (i) a bi-functional fusion protein comprising a sialic acid-binding domain (SBD) and a biotin-binding moiety (BBM), and (ii) comprises at least one biotinylated polysaccharide antigen from Group B Streptococcus (GBS) or Streptococcus agalactiae, where the biotinylated GBS polypeptide comprises sialic acid molecules (e.g., see FIG. 2B). Also demonstrated herein in the Examples is the generation of a MAPS-X immunogenic complex using a bi-functional fusion protein comprising a sialic acid-binding domain (SBD) and a biotin-binding moiety (BBM), where the MAPS-X immunogenic complex further comprises at least one biotinylated polysaccharide from Streptococcus pneumoniae, where the biotinylated Streptococcus pneumoniae polysaccharide does not comprise sialic acid molecules.

Additionally, the inventors also demonstrate herein the generation of a MAPS-X immunogenic complex using a bi-functional fusion protein comprising a sialic acid-binding domain (SBD) and a biotin-binding moiety (BBM), where the bi-functional fusion protein further comprises at least one polypeptide from GBS.

In some embodiments, it is envisioned that any MAPS-X immunogenic complex can be modified and cross-linked with a bifunctional SBD-BBM fusion protein as disclosed herein, including a SBD-Antigen fusion protein as disclosed herein, for example any MAPS-X immunogenic complex as disclosed in in U.S. Pat. No. 10,766,932, and other Multiple presenting antigen systems (MAPS), such as those disclosed in in International Applications: WO/2020/056127, WO/2020/056202, WO/2014/124228, WO/2023/039223, U.S. Ser. No. 11/560,410B2, WO/2018/183475, WO/2018/217564, WO/2023/102359, WO/2023/102359A9, WO/2012/155007, WO/2023/192997A2WO/2013/134656, WO/2023/039108, WO/2012/155053, WO/2023/172741A2, WO/2014/124228, WO/2020/056127, WO/2017/192801, WO/2020/056202, WO/2018/237221, WO/2023/039223, U.S. Ser. No. 11/305,001, US20220362367, US20210008192, US20200121777, US20140154287, U.S. Ser. No. 10/766,932, US20210332090, US20230233667, U.S. Ser. No. 11/560,410, U.S. Ser. No. 10/611,805, US20160090404, U.S. Ser. No. 10/017,548, U.S. Pat. No. 9,499,593, US20140154286, US20200407404, US20190119335, US20150374811, U.S. Ser. No. 11/576,958, US20230081705, US20230089151, US20200087361, US20190119332, U.S. Ser. No. 11/013,793, U.S. Ser. No. 11/701,416, US20210346487, US20200222522, U.S. Ser. No. 11/612,647, US20220072118, US20230091255, or US20150374811, or WO2023/102359 or PCT applications: PCT/2023/76822 and PCT/2023/76878, each of which are incorporated herein in their entirety by reference. In some embodiments, the bifunctional SBD-BBM fusion can also be used in a composition with other polysaccharide based vaccines, where the antigenic polysaccharide is from any bacteria known to a skilled artisan that has sialic acid on the surface of the polysaccharide.

In all aspects of the embodiments disclosed herein the BBM is Rhizavidin and comprises an amino acid sequence having at least 80%, or at least 85%, or at least 90%, or at least 95% sequence identity to SEQ ID NO: 1 or SEQ ID NO:2. In some embodiments, the SBD comprises an amino acid sequence having at least 80%, or at least 85%, or at least 90%, or at least 95% sequence identity to SEQ ID NO: 2. In some embodiments, the bi-functional fusion protein comprises an amino acid sequence having at least 80%, or at least 85%, or at least 90%, or at least 95% sequence identity to SEQ ID NO: 3.

Another aspect of the present invention relates to, a MAPS-X immune composition comprising at least one species immunogenic complex, wherein each species of the MAPS-X immunogenic complex comprises; (i) at least a first biotinylated polysaccharide antigen (PS1), comprising at least one sialic acid molecule and at least one biotin molecule, (ii) at least a second biotinylated polysaccharide antigen (PS2), optionally comprising at least one sialic acid molecule and at least one biotin molecule, and (iii) at least one fusion protein, each fusion protein comprises at least a biotin-biotin moiety (BBM) and a sialic acid binding moiety (SBD), and wherein a BBM of at least one fusion protein non-covalently associates with at least one biotin molecule on the PS1 and the SBD non-covalently associates with at least one sialic acid on the PS2, and/or wherein a SBD of at least one fusion protein non-covalently associates with at least one sialic acid molecule on the PS1 and the BBM non-covalently associates with at least biotin molecule on the PS2, and wherein the PS1, PS2 and the fusion protein form a MAPS-X immunogenic complex by the non-covalent association of at least one fusion protein to both the first biotinylated polysaccharide antigen and the second biotinylated polysaccharide antigen.

In some embodiments, the immunogenic complex can comprise the following non-covalent associations; PS1:BBM-SBD:PS2, where : represents a non-covalent interation and where the BBM of the SBD-BBM fusion protein non-covalently associates with a biotin on the first polysaccharide (PS1), and the SBD of the SBD-BBM fusion protein non-covalently associates with a sialic acid on the second polysaccharide antigen (PS2), to form an MAPS-X immunogenic complex. In some embodiments, the immunogenic complex can comprise the following non-covalent associations: PS1-:BBM-SBD:PS2, where the SBD of the SBD-BBM fusion protein non-covalently associates with a sialic acid on the first polysaccharide antigen (PS1), and the BBM of the SBD-BBM fusion protein non-covalently associates with a biotin on the second polysaccharide antigen (PS2), to form a MAPS-X immunogenic complex. As the first (PS1) and second antigenic polysaccharide antigens (PS2) can both have biotin molecules and sialic acid molecules on their surface, the immune complex can comprise both non-covalent associations: PS1-:BBM-SBD:PS2 and PS1:SBD-BBM:PS2 (see, e.g., FIG. 1B).

In some embodiments, the PS1 and PS2 for each immunogenic complex of each species is from the same bacterial serotype. In some embodiments, the PS1 and PS2 for each immunogenic complex for each species is from the different bacterial serotypes.

Another aspect of the present invention relates to, methods of making or producing the immune composition as disclosed herein, a pharmaceutical composition comprising the immune compositions as disclosed herein and/or a fusion protein disclosed herein, a vaccine, e.g., a multivalent vaccine, comprising the immune composition as disclosed herein, a method to induce an immune response in a subject by administering any one or more of: the immune compositions as disclosed herein and/or a fusion protein as disclosed herein, wherein the immune response is an antibody or B cell response.

### BRIEF DESCRIPTION OF THE DRAWINGS

FIG. 1A-1F are schematic drawings that show an exemplary MAPS-X immunogenic complexes using the bi-functional SBD-BBM fusion protein, including, but not limited to a SBD-[GBS-Ag]n-BBM fusion protein. FIG. 1A is a schematic drawing showing the generation of a MAPS composition comprising a rhizavidin-antigen fusion protein which non-covalently associates with a biotinylated polysaccharide in an existing MAPS complex as disclosed in 10,766,932. FIG. 1B is an exemplary MAPS-X immunogenic complex disclosed herein, comprising 5 bifunctional Rhavi-SBD fusion proteins that join two biotinylated polysaccharide antigens (PS1 and PS2), where the BBM-SBD fusion proteins function as a cross-linking intermediate molecules. Additionally, the SBD can bind to the sialic acid on the antigenic polysaccharides to prevent the sialic acid-mediated immune suppression, as well as to enlarge the MAPS-X complex. Moreover, in the embodiment shown in FIG. 1B a cross-linked multiple antigen presenting system (MAPS-X) complex comprises at least a first polysaccharide antigen (PS1) and a second polysaccharide antigen (PS2) and at least 5 SBD-BBM fusion proteins, where the SBD of a first fusion protein interacts with and non-covalently associates to sialic acid on a first polysaccharide antigen (PS1), and the Rhavi of the same first fusion protein interacts with and non-covalently associates to a biotin located on a second polysaccharide antigen (PS2), and vice versa (e.g., the Rhavi of a second fusion protein interacts with and non-covalently associates to biotin on a first polysaccharide antigen (PS1), and the SBD of the same second fusion protein interacts with and non-covalently associates, or binds to a sialic acid located on a second polysaccharide antigen (PS2) to form a MAPS-X immunogenic complex. In some embodiments, the PS1 and PS2 can be the same or different pathogenic polysaccharides, for example, the PS1 and PS2 can be selected from one of the serotypes of the same pathogen, or different pathogens. In some embodiments, the SBD-BBM can further comprise one, or two polypeptide antigens. FIG. 1C is a schematic showing exemplary embodiments of a MAPS-X immunogenic complex, showing (i) a single bifunctional SBD-BBM fusion protein non-covalently associating with the PS1 and PS2, (ii) two (or a plurality of) bifunctional SBD-BBM fusion proteins non-covalently associating with the PS1 and PS2, and (iii) the SBD-BBM can optionally comprise a polypeptide antigen, and shows an exemplary MAPS-X with two (or a plurality of) different species of bifunctional SBD-[Ag]-BBM fusion proteins non-covalently associating with the PS1 and PS2. FIG. 1D is a schematic showing exemplary embodiments of MAPS-X immunogenic complex species, with FIG. 1D(i) showing the MAPS-X complex of FIG. 1C(ii) or FIG. 1C(iii) further comprising a plurality of BBM-[Ag] fusion proteins (which can be the same or different species), which can non-covalently associate with one PS. FIG. 1D(ii) shows a MAPS-X complex of FIG. 1C(ii) or FIG. 1C(iii) further comprising a plurality of SBD-[Ag] fusion proteins (which can be the same or different species), which can non-covalently associate with only one PS at a time. FIG. 1D(iii) shows the MAPS-X complex of FIG. 1C(ii) or FIG. 1C(iii) further comprising a plurality of both BBM-[Ag] fusion proteins and SBD-[Ag] fusion proteins, where these non-bifunctional fusion proteins (e.g., only bind to one polysaccharide at a time) can comprise the same polypeptide antigen, or a different polypeptide antigen to that present in the bifunctional SBD-[Ag]-BBM fusion protein. FIG. 1E is a schematic showing an exemplary embodiment of MAPS-X immunogenic complex species, which forms a larger complex as compared to a MAPS complex that does not comprise a bifunctional SBD-BBM fusion protein. For illustrative purposes only, shown is a MAPS-X complex of 5 polysaccharide antigens (PS1-PS5), a first SBD-BBM fusion protein (FP1) can associate with PS1 and PS2, and a second SBD-BBM fusion protein (FP2) can associate with PS2 and PS3, and a third SBD-BBM fusion protein (FP3) can associate with, e.g., PS3 and PS4, and a fourth SBD-BBM fusion protein (FP2) can associate with, e.g., PS4 and PS5, etc. thereby forming a multi-bifunctional fusion protein and GBS polysaccharide complex (e.g., a MAPS-X immunogenic complex). Some SBD-BBM fusion proteins can also associate with polysaccharide antigens already existing in the complex, for example, a fifth SBD-BBM fusion protein (FP5) can associate with PS5 and PS1, a sixth SBD-BBM fusion protein (FP6) can associate with PS5 and PS3, strengthening the MAPS-X complex. The polysaccharide antigens, e.g., PS1-PS5 can be from the same pathogen, and be the same subtype, and or can be from different pathogens. In alternative embodiments, each PS (e.g., PS1, PS2, PS3, PS4, PS5, PS6 etc.) can be from a different subtype of the same pathogen. Accordingly, due to the presence of a plurality of bifunctional SBD-BBM fusion proteins which can non-covalently associate with two polysaccharide antigens, a MAPS-X immunogenic complex as disclosed herein can cross-link a plurality of polysaccharides in the complex, therefore forming larger complexes than prior MAPS complexes which comprise biotin-binding fusion proteins that can non-covalently associate with only one polysaccharide at a time (e.g., see FIG. 1A). FIG. 1F is a schematic showing an exemplary embodiment of a 3 valent (3V) MAPS-X vaccine composition comprising three different MAPS-X immunogenic complex species, each species comprising a different biotinylated polysaccharide. Each species of the MAPS-X immunogenic complex can comprise the same, or a different SBD-BBM (including the presence of a polypeptide antigen (e.g., a SBD-[GBS-Ag]-BBM fusion protein) and can be a distinct MAPS-X complex to other species of MAPS-X immunogenic complex in the vaccine composition. Additionally, each MAPS-X immunogenic complex within the composition can comprise the same species of SBD-[Ag]-BM fusion protein, or different species within the complex (e.g., see FIG. 1C(ii) and 1C(ii)). Additionally, each MAPS-X can also further comprise at least one BBM-Ag and/or at least one SBD-Ag fusion proteins (not shown).

FIG. 2A-2B are schematic drawings that shows an exemplary MAPS-X vaccine using the bi-functional fusion protein comprising a SBD-BBM fusion protein that further comprises a polypeptide antigen (e.g., the MAPS-X complex comprises a SBD-[Antigen]n-BBM fusion protein). FIG. 2A shows an exemplary SBD-fusion protein comprising a Rhizavidin-antigen-SBD fusion protein and a biotinylated polysaccharide, e.g., a biotinylated GBS polysaccharide. GBS capsular polysaccharides comprise sialic acids, which are optimal for capsule polymerization and expression, as well as an epitope for opsonic antibodies. FIG. 2B shows the formation of a multiple antigen presenting system (MAPS) complex comprising at least a first polysaccharide antigen (PS1) and a second polysaccharide antigen (PS2) and at least two SBD-[Antigen]-BBM fusion proteins, where the SBD of a first fusion protein interacts with and non-covalently binds to sialic acid on a first polysaccharide antigen (PS1), and the Rhavi of the same first fusion protein interacts with and non-covalently binds to a biotin located on a second polysaccharide antigen (PS2), and vice versa (e.g., the Rhavi of a second fusion protein interacts with and non-covalently binds to biotin on a first polysaccharide (PS1), and the SBD of the same second fusion protein interacts with and non-covalently binds to a sialic acid located on a second polysaccharide antigen (PS2)) to form a MAPS-X immunogenic complex. In some embodiments, an SBD-[Antigen]-BBM fusion protein can comprise one, or two, or 3, or 4 or 5 or more than 5 polypeptide antigens, as disclosed herein.

FIG. 3A-3B shows exemplary data of the incorporation of the Rhavi-SBD fusion protein significantly enhances antibody responses to GBS polysaccharides from serotypes Ib, II and III. The data shows utilization of the SBD to reversibly shield the sialic acid molecule on GBS polysaccharide during immunization. FIG. 3A is a schematic drawing showing that the Rhavi-SBD protein will bind to/shield sialic acid molecules on GBS PS. The Rhavi-SBD fusion protein will create a cross-linking effect that both further blocks the access of the unbound sialic acid molecules to cell surface receptors and enlarges the size of the final MAPs complexes. In FIG. 3B, it shows the results of complex formation on a Superdex200 10/30 column. Comprising the same polysaccharide, rhavi-SBD MAPS complex (e.g., MAPS-X) (black curve) has a larger size (indicated by the position of the elution peak) and more compact structure (indicated by the width of the elution peak) compared to rhavi MAPS (red curve) or rhavi MAPS associated with SBD protein (blue curve).

FIG. 4. shows that incorporation of the bifunctional Rhavi-SBD fusion protein significantly enhances antibody responses to GBS polysaccharides from serotypes Ib, II and III, as opposed to Rhavi-antigen.

FIG. 5 shows that the bifunctional Rhavi-SBD fusion protein, or SBD a stand-along protein enhances anti-GBS polysaccharide responses for GBS subtypes 1b, II, and III.

FIG. 6 shows anti-GBS polysaccharide antibody response of the MAPS complex comprising an SBD-[Ag]-BBM fusion protein as compared to a Rhavi-antigen fusion protein, demonstrating that incorporation of SBD in the SBD-[Ag]-BBM fusion carrier protein significantly enhances antibody to GBS polysaccharide. The antibody response to GBS polysaccharides from serotypes Ib, II and III polysaccharides (GBS1b, GBSII or GBSIII) was assessed in a MAPS complex comprising Rhavi-antigen (e.g., a fusion protein comprising Rhavi-0435 antigen) or a MAPS-X complex comprising a SBD-[Antigen]-BBM fusion protein (e.g., Rhavi-0435-SBD fusion protein), showing that incorporation of Rhavi-(antigen)-SBD fusion protein in the MAPS complex, as opposed to Rhavi-antigen significantly enhances antibody responses to the GBS polysaccharides.

FIG. 7 shows the antibody response to pneumococcal polysaccharides from serotypes 1, 14, and 19A after immunization with a MAPS-X complex comprising the SBD-Rhavi fusion protein comprising the 0435 polypeptide antigen, as compared to a MAPS complex comprising Rhavi-0435 fusion protein. It shows that MAPS-X complex, compared to the original MAPS complex, induces significantly enhanced antibody responses to pneumococcal polysaccharides.

FIG. 8 shows immunization with 6V MAPS-X immunogenic composition induces a robust functional antibody response to GBS polysaccharides from serotypes Ia, Ib, II, III, V and VII. Right: OPK (opsonic killing assay) of anti-GBS antibodies to specific antigen polypeptides. Rabbits were immunized with a 6V Rhavi-SBD MAPS vaccine (0.4 g PS/dose). OPK titer of pre-immune sera is below the lower limit of detection (20) for all serotypes.

FIG. 9 shows the OPK (opsonic killing assay) of anti-GBS antibodies to specific antigen polypeptides PI-2a, Sip, Rib, AlpC and Alp1-3 on serotypes Ia, Ib, II, III, V and VII of Streptococcus agalactiae.

FIG. 10 shows that P1 sera from rabbits immunized with Rhavi-[Sip-Rib]-SBD MAPS-X have enhanced killing activity against GBS serotypes II (strains 28 and SA9), III and V.

FIG. 11 shows that mice immunized with 7V MAPS-X immunogenic composition induced an anti-polysaccharide IgG immune response to polysaccharides from serotypes Ia, Ib, II, III, IV, V and VII of Streptococcus agalactiae.

FIG. 12 shows mice administered the 7V MAPS-X immunogenic composition induced a robust immune response to Rib and Sip proteins, as determined by the anti-Rib and anti-Sip IgG responses.

### DETAILED DESCRIPTION OF CERTAIN EMBODIMENTS

The present disclosure relates, generally, to compositions, systems, and methods of use thereof, of herein referred to a “MAPS-X immunogenic complex”, The technology relates to a cross-linked MAPS immunogenic complex, which comprises a bifunctional fusion protein which non-covalently associates with two polysaccharide antigens in the complex, therefore forming a large immunogenic complex where 2 or more polysaccharide antigens are connected via the bifunctional fusion protein. In all aspects of the technology disclosed herein, a bifunctional fusion protein that connects the two polysaccharides comprises at least (i) a biotin binding moiety (BBM) and (ii) a sialic acid binding domain (SBD) as disclosed herein, and is referred to herein as a “SBD-BBM fusion” protein. Additionally, an added advantage of the presence to the bifunctional SBD-BBM fusion protein is that the sialic acid-binding domain (SBD) can reduce or overcome the immune suppression of native sialic acid on the surface of bacterial or other pathogenic polysaccharides.

Accordingly, herein the inventors have developed a MAPS-X immunogenic complex where two or more biotinylated polysaccharide antigens are interconnected or joined together by one or more SBD-BBM fusion proteins. As disclosed herein, the biotinylated polysaccharide antigens can be on the same polysaccharide macromolecule, therefore enabling intra-macromolecule linking of polymer polysaccharide chains, and/or in some embodiments, the polysaccharide antigens are on different (i.e., distinct) polysaccharide macromolecules, therefore enabling inter-macromolecule linking of polymer chains from different polysaccharide macromolecules.

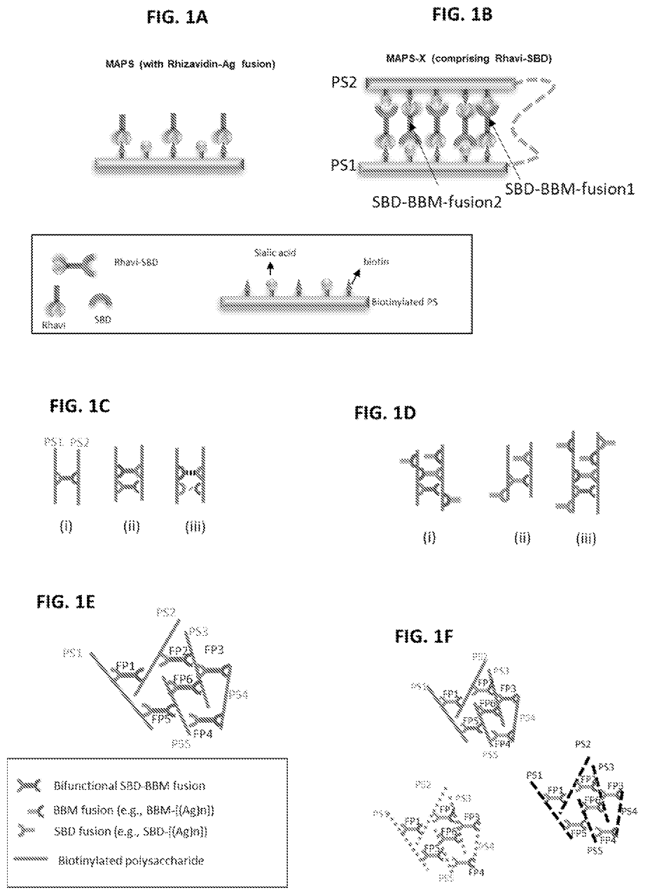

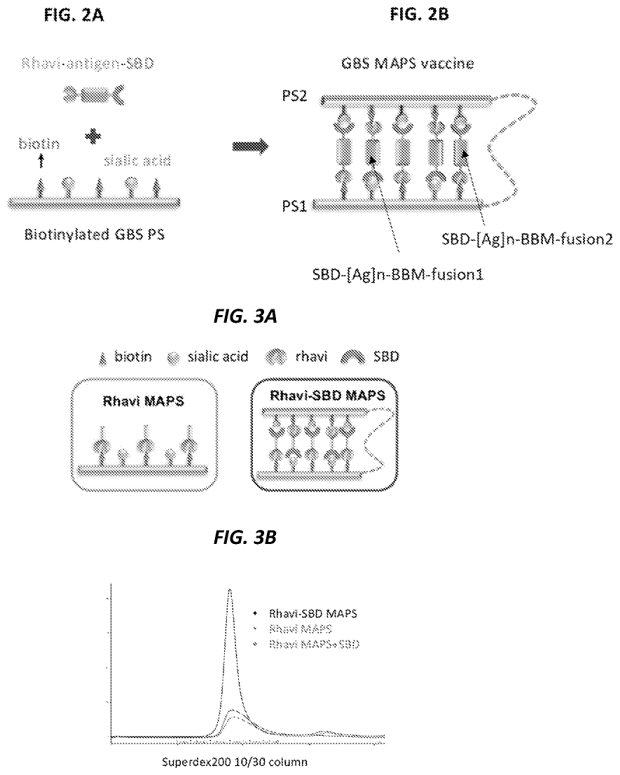

The present disclosure relates, generally, to compositions, systems, and methods of use thereof, of a MAPS-X immunogenic complex. Some aspects of the technology relate to compositions, including, a vaccines, comprising a plurality of MAPS-X immunogenic complexes, wherein each MAPS-X immunogenic complex comprises: (a) at least one biotinylated polysaccharide antigen; and (b) at least one bifunctional SBD-BBM fusion protein; and wherein the biotinylated polysaccharide antigen is non-covalently associated with the least one SBD-BBM fusion protein to form a MAPS-X immunogenic complex. In some embodiments, the MAPS-X immunogenic complex comprises at least two biotinylated polysaccharide antigens, e.g. PS1 and PS2, e.g., as illustrated in an exemplary MAPS-X in FIG. 1B.

Such complexes can be used, e.g., to induce and/or increase an immunoprotective response in subjects at risk of or suffering from an infection from a pathogen of interest, e.g., a pathogen from which the polysaccharide is derived.

Accordingly, aspects of the technology disclosed herein relates to methods, compositions and vaccines comprising a MAPS-X immunogenic complex using a bi-functional fusion protein comprising a sialic acid-binding domain (SBD) and a biotin-binding moiety (BBM), where the bi-functional fusion protein further comprises at least one antigenic polypeptide, as disclosed herein.

### I. MAPS-X Immunogenic Complexes

Aspects of the technology relate to a MAPS-X immunogenic complex, wherein each MAPS-X immunogenic complex comprises: (a) a first biotinylated antigenic polysaccharide antigen (PS1); and (b) at least one bifunctional SBD-BBM fusion protein, and wherein the biotinylated polysaccharide antigen is non-covalently associated with the biotin on at least one SBD-BBM fusion proteins form an immunogenic complex.

For illustrative purposes only, referring to FIG. 1B, a MAPS-X immunogenic complex comprises; (i) at least a first biotinylated polysaccharide antigen (PS1) comprising at least one biotin molecule, (ii) at least a second biotinylated polysaccharide antigen (PS2), wherein PS2 can optionally comprise at least one biotin molecule, or at least one sialic acid molecule, or both, and (iii) at least one bifunctional SBD-BBM fusion protein (e.g., SBD-BBM fusion protein).

In some embodiments, for illustrative purposes only, where at least two bifunctional SBD-BBM fusion protein are used, a BBM of at least a first fusion protein (SBD-BBM fusion1) non-covalently associates with at least one biotin molecule on the first biotinylated polysaccharide antigen (PS1) and the SBD non-covalently associates with, e.g., at least one sialic acid on the second biotinylated polysaccharide antigen (PS2), and the SBD of a second SBD-BBM fusion protein (SBD-BBM fusion2) non-covalently associates with at least one sialic acid molecule on the first biotinylated polysaccharide antigen (PS1) and the BBM of the second fusion protein non-covalently associates with at least biotin molecule on the second biotinylated polysaccharide antigen (PS2). This results in the PS1 and PS2 forming a MAPS-complex by the non-covalent association via the first and second bifunctional SBD-BBM fusion proteins.

Referring to FIG. 1B tor illustrative purposes only, another aspect of the present invention relates to methods, compositions and vaccines comprising a MAPS-X immunogenic complex, wherein a composition at least one species of MAPS-X immunogenic complex, wherein each species of the MAPS-X immunogenic complex comprises; (i) at least a first biotinylated polysaccharide antigen (PS1) comprising at least one biotin molecule, (ii) at least a second polysaccharide antigen (PS2), and (iii) at least one SBD-BBM bifunctional fusion protein as disclosed herein, each fusion protein comprises at least a biotin-biotin moiety (BBM) and a sialic acid binding domain (SBD), and wherein a BBM of at least one SBD-BBM fusion protein non-covalently associates with at least one biotin molecule on the first biotinylated polysaccharide antigen (PS1) and the SBD non-covalently associates with the PS2 (e.g., if a sialic acid is present, the SBD can associate at least one sialic acid on the second biotinylated polysaccharide antigen (PS2)), therefore joining the PS1 and PS1 via the association with the SBD-BBM fusion proteins(s) serving as a cross-linking intermediate. In some embodiments, PS1 and PS2 have sialic acids. In some embodiments, PS1 and PS2 are both biotinylated but do not have sialic acids on.

Optionally, where the MAPS-X complex comprises more than one bifunctional SBD-BBM fusion proteins, e.g., a 2^{nd} a bifunctional SBD-BBM fusion protein (FP2), a SBD of at least a second fusion protein (FP2) non-covalently associates with at least one sialic acid molecule on the first biotinylated polysaccharide antigen (PS1) and the BBM of this second fusion protein non-covalently associates with at least biotin molecule on the second biotinylated polysaccharide antigen (PS2), therefore further joining the PS1 and PS1 via the association with the SBD-BBM fusion proteins(s) serving as a cross-linking intermediate.

For illustrative purposes only, referring to FIG. 1E shows an exemplary embodiment of MAPS-X immunogenic complex species, which forms a larger complex as compared to a MAPS complex that does not comprise a bifunctional SBD-BBM fusion protein. For illustrative purposes only, shown is a MAPS-X complex of 5 polysaccharides (PS1-PS5), a first SBD-BBM fusion protein (FP1) can associate with PS1 and PS2, and a second SBD-BBM fusion protein (FP2) can associate with PS2 and PS3, and a third SBD-BBM fusion protein (FP3) can associate with, e.g., PS3 and PS4, and a fourth SBD-BBM fusion protein (FP2) can associate with, e.g., PS4 and PS5, etc. thereby forming a MAPS-X immunogenic complex, where the polysaccharides are joined or cross-linked via the SBD-BBM fusion protein intermediate. Some SBD-BBM fusion proteins can also associate with polysaccharides already existing in the complex, for example, a fifth SBD-BBM fusion protein (FP5) can associate with PS5 and PS1, a sixth SBD-BBM fusion protein (FP6) can associate with PS5 and PS3, strengthening the MAPS-X complex. The polysaccharides, e.g., PS1-PS5 be from the same subtype (or serotype) of bacteria or pathogen. In alternative embodiments, each PS (e.g., PS1, PS2, PS3, PS4, PS5, PS6 etc.) can be from a different subtypes of bacteria or pathogen. Accordingly, due to the presence of a plurality of bifunctional SBD-BBM fusion proteins which can non-covalently associate with two polysaccharide antigens, a MAPS-X immunogenic complex as disclosed herein can cross-link a plurality of polysaccharide antigens in the complex, therefore forming larger complexes than prior MAPS complexes which comprise biotin-binding fusion proteins that can non-covalently associate with only one polysaccharide antigen at a time.

It is also envisioned that the SBD-BBM fusion protein further comprises at least one, or at least 2, or at least 3 or more antigenic polypeptide antigens, and can optionally be located between a BBM and a SBD polypeptide, or in any order.

In some embodiments where both the first (PS1) and second antigenic polysaccharides (PS2) have both biotin molecules and sialic acid molecules on their surface, the MAPS-X immune complex can comprise the following non-covalent associations: PS1-(SBD-BBM fusion protein1)-PS2 and PS1-(SBD-BBM fusion protein2)-PS2 (see, e.g., FIG. 1B and FIG. 1E). It is envisioned that the SBD-BBM fusion protein further comprises at least one, or at least 2, or at least 3 or more antigenic polypeptide antigens. The position of a pathogen polypeptide antigen in the fusion protein is flexible, and can optionally be located anywhere between a BBM and a SBD polypeptide, or at the N- or C-terminus, as disclosed in the fusion proteins listed in Table 3. It is envisioned that as a plurality of SBD-BBM fusion protein are used in each MAPS-X complex, each polysaccharide can be non-covalently attached to more than 1 (e.g., 1, 2, 3, 4, 5, 6, 7, 8 or more) different SBD-BBM fusion proteins, therefore cross-linking the polysaccharides in the complex via the bifunctional SBD-BBM fusion proteins. Accordingly, due to the presence of a plurality of bifunctional SBD-[Ag]-BBM fusion proteins, each of which can non-covalently associate with two polysaccharide antigens, a MAPS-X immunogenic complex as disclosed herein can cross-link a plurality of polysaccharides in the MAPS-X immunogenic complex, forming larger complexes than prior MAPS complexes which comprise biotin-binding fusion proteins that can non-covalently associate with only one polysaccharide at a time. Such cross-linking and generation of larger MAPS-X immunogenic complex induces a larger immunological response to both the polysaccharide and/or GBS antigen on administration to a subject.

### II. Bifunctional SBD-BBM Fusion Proteins

### a) SBD Proteins

In some embodiments, a SBD-BBM fusion protein comprises a sialic acid binding domain (SBD). In some embodiments, a SBD-BBM fusion protein of a MAPS-X immunogenic complex comprises a SBD, and one or more polypeptide antigens. In some embodiments, a SBD-BBM fusion protein comprises a SBD and two or more polypeptide antigens.

As used herein, a “sialic acid binding domain (SBD)” or “sialic acid binding molecule (SBM)” are used interchangeably, and refers to a portion, fragment of variant of a sialic acid binding protein that binds or has affinity for a sialic acid moiety on the surface of a polysaccharide. It should be understood that any polypeptide or molecules which exhibit an affinity for sialic acid, bind to or otherwise couple to or associate with sialic acid moieties is encompassed in the term SBD. Thus the term “sialic acid binding molecule” may encompass any fragment, which retains an ability to bind to or otherwise couple or associate with a sialic acid moiety.

In some embodiments, a bifunctional SBD-BBM fusion protein useful in the MAPS-X immunogenic complex as disclosed herein comprises, in any order (i) at least one BBM and (ii) a SBD. A SBD of a bifunctional fusion protein as disclosed herein may comprise a single molecule capable of binding sialic acid (a monomeric or monovalent molecule, for example) or, alternatively, two or more sialic acid binding molecules (which may all be the same or different—a polymeric or multivalent molecule, for example).

In some embodiments, a SBD-BBM fusion protein comprises one or more of the polypeptides listed in Table 1.

TABLE 1
Exemplary Polypeptide Components
of SBD-BBM Fusion Proteins.

| | Amino Acid | |
|---|---|---|
| | SEQ ID NO | DNA SEQ |
| Component | numbers | ID NO: |
| Rhavi | 1 | 78 |
| Rhavi(A5T) | 2 | |
| SBD1 NanA (121-305) | 3 | 80 |
| SBD2 NanL (81-272) | 4 | 81 or 82 |
| | | (codon |
| | | optimized) |
| SBD3 NanB (40-230) | 5 | 83 |
| SBD4 NanC 82-270 | 6 | 84 |
| NanH (Salmonella) | 7 | |
| NanH2(Salmonella) | 8 | |
| NanH3 Trypanosoma cruzi | 9 | |
| 4-399 | | |
| SBD NanA (full-length) | 10 | |
| NanH (Vibrio cholera) | 11 | |
| NanH-Truncated (25-216aa) | 12 | |
| (Vibrio cholera) | | |
| Rhavi-SBD1 | 13 | |
| Rhavi-SBD2 | 14 | |
| Rhavi-SBD3 | 15 | |
| Rhavi-SBD4 | 16 | |
| SBD1-Rhavi | 17 | |
| SBD2-Rhavi | 18 | |
| SBD3-Rhavi | 19 | |
| SBD4-Rhavi | 20 | |

In some embodiments, a SBD-BBM fusion protein useful in the MAPS-X immunogenic complex as disclosed herein comprises, in any order (i) at least one BBM and (ii) SBD selected from any of SBD1, SBD2, SBD3, SBD4, NanH, NanH2, NanH3, VcNanH, or having an amino acid sequence with at least 80%, or at least 85%, or at least 90%, or at least 95% sequence identity to any of: SEQ ID NOs: 3-12.

In some embodiments, a SBD-BBM fusion protein useful in the MAPS-X immunogenic complex as disclosed herein comprises, or consists essentially of, in any order (i) at least one BBM and (ii) SBD having an amino acid sequence with at least 80%, or at least 85%, or at least 90%, or at least 95% sequence identity to SEQ ID NO: 3 (SBD1). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 4 (SBD2). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 5 (SBD3). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 6 (SBD4). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 7 (NanH-Salmonella). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 8 (Nan-H2 Salmonella). In some embodiments, a SBD-BBM fusion protein useful in the MAPS-X immunogenic complex as disclosed herein comprises, in any order (i) at least one BBM and (ii) a SBD having an amino acid sequence with at least 80%, or at least 85%, or at least 90%, or at least 95% sequence identity to SEQ ID NO: 9 (Nan-H3 Salmonella). In some embodiments, a SBD-BBM fusion protein useful in the MAPS-X immunogenic complex as disclosed herein comprises, in any order (i) at least one BBM and (ii) a SBD having an amino acid sequence with at least 80%, or at least 85%, or at least 90%, or at least 95% sequence identity to SEQ ID NO: 12 (NanH-truncated Vibro Cholera). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 9 (Nan-H3 Trypanosoma Cruzi). In some embodiments, a SBD-BBM fusion protein useful in the MAPS-X immunogenic complex as disclosed herein comprises, in any order (i) at least one BBM and (ii) SBD having an amino acid sequence with at least 80%, or at least 85%, or at least 90%, or at least 95% sequence identity to SEQ ID NO: 11 or SEQ ID NO: 12 (Nan-H Vibrio cholera).

The term “sialic acid” as used herein, embraces all forms of N- or O-substituted neuraminic acid and includes all synthetic, naturally occurring and/or modified forms thereof. Sialic acids may be found as components of cell surface molecules, glycoproteins and glycolipids. Sialic acids are present at the end (terminal regions) of sugar chains connected to cell membranes and/or proteins. The sialic acid family encompasses a number (approximately 50) of derivatives that may result from acetylation, glycosylation, lactonisation and methylation at C4, C5, C7, C8 and C9. All such derivatives are to be embraced by the term “sialic acid”. Sialic acids are found linked a(2,3) or a(2,6) to Gal and GalNAc or a(2,8) or a(2,9) to another sialic acid. Accordingly, while the term “sialic acid” is used throughout this specification, it encompasses all derivatives, analogues or variants (either naturally occurring or synthetically generated) thereof as well as monomers, dimers, trimers, oligomers, polymers or concatamers comprising the same.

### Sialic Acid Binding Domain (SBD):

A sialic acid binding domain (SBD) useful as a component of a SBD-BBM fusion protein as disclosed herein exhibits an affinity for sialic acid-including all forms of sialic acid described above and, in particular sialic acid present on the surface of mammalian cells. For example, the molecules of this invention may exhibit an affinity for cell membrane receptors, which comprise sialic acid. Cell receptors of this type may be present on the surface of epithelial cells-including epithelial cells of the mucosal and respiratory tracts. A number of pathogens, including viral and/or bacterial pathogens may express molecules, which exhibit an affinity for sialic acid. Pathogens of this type have evolved to exploit cell surface sialic acid moieties as a means to bind to host cells. Once bound to a cell via, for example a host cell surface bound sialic acid moiety, a pathogen may colonise the cell surface and/or infect/enter the cell.

In particular embodiments, a sialic acid binding domain (SBD) that is a component of a SBD-BBM fusion protein as disclosed herein exhibits affinity for sialic acid present on an antigenic polysaccharide. In particular embodiments, a sialic acid binding domain (SBD) that is a component of a SBD-BBM fusion protein as disclosed herein exhibits affinity for an antigenic polysaccharide that does not comprise a sialic acid, wherein the antigenic polysaccharide has a negative polarity, and/or has a similar polarity to S. pneumococci polysaccharide.

In some embodiments, a SBD of a SBD-BBM fusion protein as disclosed herein may exhibit an affinity for α-2,6-linked sialic acid predominantly present on cells of the human upper respiratory tract. Additionally or alternatively, the sialic acid binding domains may exhibit an affinity for α-2,3-linked sialic acid present on cells of the upper and lower respiratory tracts.

### Exemplary SBDs:

A SBD of a SBD-BBM fusion protein as disclosed herein can comprise one or more moieties that exhibit an affinity for sialic acid.

SBD1 (NanA): In some embodiments, an exemplary SBD for use in a SBD-BBM fusion protein is a fragment of Streptococcus pneumoniae NanA sialidase (NanA), where the amino acid sequence is SEQ ID NO: 3 (1035 amino acids) and has been deposited under accession number P62575. In some embodiments, a SBD of a SBD-BBM fusion protein as disclosed herein comprises amino acids 121-305 the full length NanA polypeptide of amino acids of SEQ ID NO: 10. In some embodiment, a SBD for use in a SBD-BBM fusion protein as disclosed herein comprises an amino acid sequence of SEQ ID NO: 3 (180aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of a SBD polypeptide of SEQ ID NO: 3. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 3. In some embodiments, a SBD polypeptide of the fusion protein is a SBD1 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 3.

SBD2 (NanL): In some embodiments, an exemplary SBD for use in a SBD-BBM fusion protein is a fragment of NanL sialidase (NanL). In some embodiments, a SBD is a fragment of amino acids 81-272 the full length NanL polypeptide. In some embodiment, a SBD for use in a SBD-BBM fusion protein as disclosed herein comprises an amino acid sequence of SEQ ID NO: 4 (192aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185 or 191 consecutive amino acids of a SBD2 polypeptide of SEQ ID NO: 4. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185 or 191 consecutive amino acids of the sequence shown in SEQ ID NO: 4. In some embodiments, a SBD polypeptide of the fusion protein is a SBD2 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185 or 191 consecutive amino acids of SBD2 amino acid sequence shown in SEQ ID NO: 4.

SBD3 (NanB): In some embodiments, an exemplary SBD for use in a SBD-BBM fusion protein is a fragment of NanB sialidase (NanB). In some embodiments, a SBD is a fragment of amino acids 40-230 the full length NanB polypeptide. In some embodiment, a SBD for use in a SBD-BBM fusion protein as disclosed herein comprises an amino acid sequence of SEQ ID NO: 5 (191aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of a SBD3 polypeptide of SEQ ID NO: 5. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of the sequence shown in SEQ ID NO: 5. In some embodiments, a SBD polypeptide of the fusion protein is a SBD3 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of SBD3 amino acid sequence shown in SEQ ID NO: 5.

SBD4 (NanC): In some embodiments, an exemplary SBD for use in a SBD-BBM fusion protein is a fragment of NanC sialidase (NanC). In some embodiments, a SBD is a fragment of amino acids 82-270 the full length NanC polypeptide. In some embodiment, a SBD for use in a SBD-BBM fusion protein as disclosed herein comprises an amino acid sequence of SEQ ID NO: 6 (189aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 190 or 192 consecutive amino acids of a SBD4 polypeptide of SEQ ID NO: 6. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 187 or 189 consecutive amino acids of the sequence shown in SEQ ID NO: 6. In some embodiments, a SBD polypeptide of the fusion protein is a SBD4 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 180, 185, 187 or 189 consecutive amino acids of SBD4 amino acid sequence shown in SEQ ID NO: 6.

NanH: In some embodiments, an exemplary SBD for use in a SBD-BBM fusion protein is a fragment of Salmonella NanH sialidase (NanH). In some embodiments, a SBD is a fragment of the full length NanH polypeptide. In some embodiment, a SBD for use in a SBD-BBM fusion protein as disclosed herein comprises NanH having an amino acid sequence of SEQ ID NO: 7 (381aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH polypeptide of SEQ ID NO: 7. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH polypeptide of SEQ ID NO: 7. In some embodiments, a SBD polypeptide of the fusion protein is a NanH immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH polypeptide of SEQ ID NO: 7.

NanH2: In some embodiments, an exemplary SBD for use in a SBD-BBM fusion protein is a fragment of Salmonella NanH2 sialidase (NanH2). In some embodiments, a SBD is a fragment of the full length NanH2 polypeptide. In some embodiment, a SBD for use in a SBD-BBM fusion protein as disclosed herein comprises NanH2 having an amino acid sequence of SEQ ID NO: 8 (404aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH2 polypeptide of SEQ ID NO: 8. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH2 polypeptide of SEQ ID NO: 8. In some embodiments, a SBD polypeptide of the fusion protein is a NanH2 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 or 380 consecutive amino acids of a NanH2 polypeptide of SEQ ID NO: 8.

NanH3: In some embodiments, an exemplary SBD for use in a SBD-BBM fusion protein is a fragment of NanH3 sialidase (NanH3) from Trypanosoma cruzi. In some embodiments, a SBD is a fragment of amino acids 4-399 the full length NanH3 polypeptide. In some embodiment, a SBD for use in a SBD-BBM fusion protein as disclosed herein comprises an amino acid sequence of SEQ ID NO: 9 (396aa), or an immunogenic variant or fragment thereof.

In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 380, 390 or 396 consecutive amino acids of a NanH2 polypeptide of SEQ ID NO: 9. In some embodiments, a SBD of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 380, 390 or 396 consecutive amino acids of a NanH2 polypeptide of SEQ ID NO: 9. In some embodiments, a SBD polypeptide of the fusion protein is a NanH3 immunogenic polypeptide and comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350 380, 390 or 396 consecutive amino acids of a NanH2 polypeptide of SEQ ID NO: 9.

Similar or homologous sialic acid binding modules present in other organisms are to be encompassed within the scope of the term “SBD” herein. In some embodiments, additional exemplary SBD for use in the fusion proteins as disclosed herein comprise the sialic acid binding domain (SBD) of Vibrio cholerae NanH sialidase (VcNanH). Accordingly, in some embodiments, an exemplary SBD for use in a SBD-BBM fusion protein is a fragment of Vibrio cholerae NanH sialidase (VcNanH sialidase), where the amino acid sequence is deposited under accession umber A5F7A4 and is as SEQ ID NO: 10 (781 amino acids). In some embodiments, a SBD region of VcNanA is from amino acid residue 25 to 216 of SEQ ID NO: 11, and corresponds to amino acid sequence SEQ ID NO: 12.

In some embodiments, a SBD for use in a SBD-BBM fusion protein is a protein or binding moiety which binds to a modified sialic acid, where modifications of sialic acids include diverse forms differing in position 5 of an amino group of neuraminic acid derivatives or an hydroxyl group of 3-deoxy-D-glycero-D-galactononulosonic acid (Kdn), different acylations of the NH₂ at position 5 (glycolyl, acetyl), and various substituent of the different hydroxyl groups including phosphate, sulfate, methyl, acetyl, etc. >50 different derivatives of sialic acids are disclosed in Table 1 of Ghosh, S. (2020). Sialic acids and sialoglycoconjugates in the biology of life, health and disease. Academic Press, which is incorporated herein in its entirety. Two most commonly expressed members of sialic acid family are Neu5Ac and Neu5Gc followed by KDN (2-keto-3-deoxy-nononic acid) and Neu (neuraminic acid). Accordingly, a SBD for use in a SBD-BBM fusion protein is a protein or binding moiety which has affinity for, and binds to any of: Neu5Ac, Neu5Gc, KDN, Neu. In some embodiments, a SBD for use in a SBD-BBM fusion protein is a protein or binding moiety which has affinity for sialic acid that have modifications to core structures of sialic acid, including modifications such as O-acetylation, O-methylation, or introduction of O-lactyl groups, sulfate, or phosphate esters at positions 4, 7, 8, and/or 9.

### (c) Rhizavidin Fusion Proteins

Rhizavidin is a naturally occurring dimeric protein in the avidin protein family, was first discovered in Rhizobium etli, a symbiotic bacterium of the common bean. Rhizavidin has only a 22% amino acid identity with chicken avidin, a protein commonly found in eggs, but with high conservation of amino acid residues involved in biotin binding [Helppolainen et al, 2007]. In some embodiments, the nucleotide sequence of rhizavidin is set forth in SEQ ID NO: 78. In some embodiments, the amino acid sequence of rhizavidin is set forth in SEQ ID NO: 1. SEQ ID NO: 1 has N-terminal 1-44 amino acids removed of the full-length protein, which are predicted to be a signal sequence(s) of rhizavidin. In some embodiments, a SBD-BBM fusion protein comprises rhizavidin. In some embodiments, a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of a rhizavidin polypeptide of SEQ ID NO: 1 or SEQ ID NO: 2.

In some embodiments, a rhizavidin polypeptide of a SBD-BBM fusion protein comprises at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 1. In some embodiments, a rhizavidin polypeptide of the fusion protein comprises an amino acid sequence that has at least 60% or more (including, e.g., at least 65%, 70%, 75%, 80%, 85%, 90%, 95%, 96%, 97%, 98%, 99%, or 100%) sequence identity to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, or 179 consecutive amino acids of the sequence shown in SEQ ID NO: 1. In some embodiments, a rhizavidin polypeptide of the fusion protein comprises an amino acid sequence of SEQ ID NO: 2, which comprises 5 different A>T amino acid modification.

In some embodiments the Rhizavidin and/or SBD of the SBD-BBM fusion protein is lipidated (i.e., a lipidated-Rhizavidin in a SBD-BBM fusion protein). Methods for lipidating Rhizavidin are disclosed in WO/2012/155053A1, and in U.S. Pat. No. 9,499,593, which are incorporated herein in its entirety by reference. Methods for purifying a Rhizavidin for use herein are disclosed in International Application number: WO/2017/192801 which is incorporated herein in its entirety by reference.

In some embodiments, a SBD-BBM fusion protein comprises a biotin-binding moiety (BBM). In some embodiments, a SBD-BBM fusion protein of the immunogenic complex comprises a biotin-binding moiety, and one or more polypeptide antigens. In some embodiments, a SBD-BBM fusion protein comprises a biotin-binding moiety and two or more polypeptide antigens. As used herein, a “biotin-binding moiety” refers to a biotin-binding protein, a biotin-binding fragment thereof, or a biotin-binding molecule thereof.

In some embodiments, the biotin-binding moiety of a SBD-BBM fusion protein comprises rhizavidin or a biotin-binding molecule or biotin-binding fragment thereof, as further described in WO 2012/155053, the contents of which are herein incorporated by reference in their entirety. In some embodiments, a biotin-binding moiety is or comprises a polypeptide having at least 80%, 85%, 90%, 95%, 96%, 97%, 98%, or 99% identity to rhizavidin, or a biotin-binding molecule or biotin-binding fragment thereof. In some embodiments, the biotin-binding moiety comprises a polypeptide of SEQ ID NO: 1 or a biotin-binding molecule or biotin-binding fragment thereof. In some embodiments, the biotin-binding moiety is or comprises a polypeptide having at least 80%, 85%, 90%, 95%, 96%, 97%, 98%, or 99% identity to the sequence of SEQ ID NO: 1 or SEQ ID NO: 2, or biotin-binding molecule or biotin-binding fragment thereof.

In some embodiments, a fusion protein useful in the MAPS-X immunogenic complex as disclosed herein comprises, in any order, (i) at least one SBD selected from any of SBD1, SBD2, SBD3, SBD4, NanH, NanH2, NanH3, VcNanH, or having an amino acid sequence with at least 80%, or at least 85%, or at least 90%, or at least 95% sequence identity to any of: SEQ ID NOs: 3-12 and (ii) a BBM which is Rhizavidin, having an amino acid sequence with at least 80%, or at least 85%, or at least 90%, or at least 95% sequence identity to SEQ ID NO: 1 or SEQ ID NO: 2.

In some embodiments, a fusion protein useful in the MAPS-X immunogenic complex as disclosed herein comprises, in any order, (i) at least one SBD having an amino acid sequence with at least 80%, or at least 85%, or at least 90%, or at least 95% sequence identity to SEQ ID NO: 2 (SBD1), and (ii) a BBM which is Rhizavidin, having an amino acid sequence with at least 80%, or at least 85%, or at least 90%, or at least 95% sequence identity to SEQ ID NO: 1 or SEQ ID NO: 2. In some embodiments, a fusion protein useful in the MAPS-X immunogenic complex as disclosed herein comprises, in any order, (i) at least one SBD having an amino acid sequence with at least 80%, or at least 85%, or at least 90%, or at least 95% sequence identity to SEQ ID NO: 4 (SBD2), and (ii) a BBM which is Rhizavidin, having an amino acid sequence with at least 80%, or at least 85%, or at least 90%, or at least 95% sequence identity to SEQ ID NO: 1 or SEQ ID NO: 2.

### Alternative Biotin-Binding Moieties to Replace Rhizavidin

In some embodiments, a SBD-BBM fusion protein described herein comprises a BBM and is a component of non-covalent Multiple Antigen Presenting System (MAPS) GBS immunogenic complexes. In some embodiments, MAPS-X complexes disclosed herein utilize the high affinity (dissociation constant [KD]≈10-15M) non-covalent binding between biotin and rhizavidin, a biotin-binding protein that has no significant predicted homology with human proteins. In some embodiments, it is envisioned that Rhizavidin polypeptide of a SBD-BBM fusion protein comprising a BBM can be readily replaced with any other biotin-binding protein.

In some embodiments, the BBM is selected from any of rhizavidin, avidin, streptavidin, bradavidin, tamavidin, lentiavidin, zebavidin, NeutrAvidin, CaptAvidin™, or a biotin-binding molecule or biotin-binding fragment thereof, or a combination thereof. In some embodiments, the biotin-binding moiety is rhizavidin, or a biotin-binding molecule or biotin-binding fragment thereof.

In some embodiments the Rhizavidin of the SBD-BBM fusion protein is lipidated (i.e., a lipidated-Rhaviavin a SBD fusion protein). In some embodiments, a rhizavidin can comprise a lipidation sequence at the N-terminus, e.g., MKKVAAFVALSLLMAGC (SEQ ID NO: 85) or an amino acid 85% identity thereto.

In some embodiments, the MAPS-X composition can further comprise a rhizavidin protein comprising SEQ ID NO: 1 or a protein with 85% sequence identity thereto, that comprises a lipidation sequence at the N-terminus, e.g., MKKVAAFVALSLLMAGC (SEQ ID NO: 86) or an amino acid 85% identity thereto, where the rhizavidin protein is not part of a SBD-BBM fusion protein or not fused to other antigen (e.g., Rhavi is not part of a Rhavi-SBD fusion protein, or Rhavi-antigen fusion protein). Lipidated rhizavidin proteins and lapidated rhizavidin fusion proteins are disclosed in US application US2016/0090404, entitled “Modified biotin-binding protein, fusion proteins thereof and applications”, which is incorporated herein in its entirety by reference. As used herein, the term “lipidated biotin-binding protein” refers to a biotin-binding protein that is covalently linked with a lipid. The lipidated biotin-binding proteins are ligands or agonists of Toll like receptor 2. Accordingly, also provided herein are methods for inducing an immune response in subject. The method comprising administering to the subject a composition comprising a lipidated biotin-binding protein.

In another aspect provided herein is a lipidated biotin-binding protein, e.g., a lipidated rhizavidin fusion protein comprising a GBS antigen for use in the MAPS-GBS immunogenic composition as disclosed herein. As used herein, the term “lipidated biotin-binding protein” refers to a biotin-binding protein that is covalently conjugated with a lipid. The lipid moieties could be a diacyl or triacyl lipid.

In some embodiments, a SBD-BBM fusion protein for use in the MAPS-X immunogenic composition as disclosed herein comprises a lipidation sequence. As used herein, the term “lipidation sequence” refers to an amino acid sequence that facilitates lipidation in bacteria, e.g., E. coli, of a polypeptide carrying the lipidating sequence. The lipidation sequence can be present at the N-terminus or the C-terminus of the protein. The lipidation sequence can be linked to the recombinant biotin-binding protein to form a fusion protein, which is in lipidated form when expressed in E. coli by conventional recombinant technology. In some embodiments, a lipidation sequence is located at the N-terminus of the biotin-binding protein.

Any lipidation sequence known to one of ordinary skill in the art can be used. In some embodiments, the lipidating sequence is MKKVAAFVALSLLMAGC (SEQ ID NO: 85) or a derivative or functional portion thereof. Other exemplary lipidating sequences include, but are not limited to, MNSKKLCCICVLFSLLAGCAS (SEQ ID NO: 86), MRYSKLTMLIPCALLLSAC (SEQ ID NO: 87), MFVTSKKMTAAVLAITLAMSLSAC (SEQ ID NO: 88), MIKRVLVVSMVGLSLVGC (SEQ ID NO: 89), and derivatives or functional portions thereof.

In some embodiments, the lipidation sequence can be fused to a SBD-BBM fusion protein via a peptide linker, wherein the peptide linker attaches the lipidating sequence to the biotin-binding protein. In some embodiment, the peptide linker comprises the amino acid sequence VSDP (SEQ ID NO: 90) or AQDP (SEQ ID NO: 91).

In some embodiments, a SBD-BBM fusion protein for use in the MAPS-X immunogenic composition as disclosed herein that is a lipoprotein as described herein have enhanced immunogenicity. Without wishing to be bound by a theory, lipid moieties at the N-terminals of the lipoproteins or lipopeptides contribute to the adjuvant activity. Accordingly, additional embodiments provide immunogenic or vaccine compositions for inducing an immunological response, comprising the isolated biotin-binding lipoprotein, or a suitable vector for in vivo expression thereof, or both, and a suitable carrier, as well as to methods for eliciting an immunological or protective response comprising administering to a host the isolated recombinant biotin-binding lipoprotein, the vector expressing the recombinant biotin-binding lipoprotein, or a composition containing the recombinant lipoprotein or vector, in an amount sufficient to elicit the response.

A MAPS-X immunogenic composition comprising a SBD-BBM fusion protein that is a lipoprotein elicits an immunological response-local or systemic. The response can, but need not, be protective.

### Exemplary SBD-BBM Fusion Proteins

In some embodiments, a SBD-BBM fusion protein useful in the MAPS-X immunogenic complex as disclosed herein comprises, in any order (i) a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 3 (SBD1). In some embodiments, a SBD-BBM fusion protein useful in the MAPS-X immunogenic complex as disclosed herein comprises, in any order (i) a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 4 (SBD2). In some embodiments, a SBD-BBM fusion protein useful in the MAPS-X immunogenic complex as disclosed herein comprises, in any order (i) at least one BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 5 (SBD3). In some embodiments, a SBD-BBM fusion protein useful in the MAPS-X immunogenic complex as disclosed herein comprises, in any order (i) at least a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 6 (SBD4). In some embodiments, a SBD-BBM fusion protein useful in the MAPS-X immunogenic complex as disclosed herein comprises, in any order (i) at least one a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 7 (NanH-Salmonella). In some embodiments, a SBD-BBM fusion protein useful in the MAPS-X immunogenic complex as disclosed herein comprises, in any order (i) at least one a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 2 (Nan-H2 Salmonella). In some embodiments, a SBD-BBM fusion protein useful in the MAPS-X immunogenic complex as disclosed herein comprises, in any order (i) at least one a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 9 (Nan-H3 Trypanosoma Cruzi). In some embodiments, a SBD-BBM fusion protein useful in the MAPS-X immunogenic complex as disclosed herein comprises, in any order (i) at least one a BBM which is Rhizavidin, having an amino acid sequence with at least 80%, or at least 85%, or at least 90%, or at least 95% sequence identity to SEQ ID NO: 1 or SEQ ID NO: 2 and (ii) SBD having an amino acid sequence with at least 80%, or at least 85%, or at least 90%, or at least 95% sequence identity to SEQ ID NO: 11 or SEQ ID NO: 12 (Nan-H Vibrio cholera).

In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 13. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 14. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 15. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 16. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 17. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 18. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 19. In some embodiments, a SBD-BBM fusion protein useful in the MAPS-X immunogenic complex as disclosed herein comprises, or consist of a fusion protein having an amino acid sequence with at least 80%, or at least 85%, or at least 90%, or at least 95% sequence identity to SEQ ID NOs: 20.

### (c) Bifunctional SBD-[GBS-Ag]-Rhavi Fusion Proteins

One aspect of the technology disclosed herein relates to a bi-functional fusion protein comprising, in any order, (i) a sialic acid-binding domain (SBD), (ii) a biotin-binding moiety (BBM), and (iii) at least 1, or at least 2, or at least 3, at least 4, at least 5, or more than 5 polypeptide antigens, e.g., polypeptide antigens from GBS, and is broadly referred to herein as SBD-[Ag]n-BBM fusion protein, where n is the number of antigenic polypeptide located between the SBD and BBM. It is envisioned that the antigen in the SBD-[Ag]n-BBM fusion protein can be located at the N-terminal, or the C-terminal of the fusion protein, or between the SBD and the BBM. For example, exemplary fusion proteins can be arranged in the following order as follows:
BBM-SBD-[Ag]n
BBM-[Ag]n-SBD
[Ag]n-BBM-SBD
[Ag]n-SBD-BBM
SBD-[Ag]n-BBM

In some embodiments, the bi-functional fusion protein comprises at least one antigenic polypeptide located between the sialic acid-binding domain (SBD) and a biotin-binding moiety (BBM), wherein the one antigenic polypeptide is from a pathogen of interest. In some embodiments, the antigenic polypeptide can be from the same pathogen as the antigenic polysaccharide in the MAPS-X immunogenic complex, e.g., if the antigenic polysaccharide is from S. pneumoniae, the polysaccharide can be from S. pneumococcus. In alternative embodiments, the antigenic polypeptide can be from a different pathogen as the antigenic polysaccharide in the MAPS-X immunogenic complex, e.g., if the antigenic polysaccharide is from Salmonella Typhi, the polysaccharide can be from a different pathogen, e.g., S. pneumoniae. In some embodiments, the bi-functional fusion protein comprises at least two antigenic polypeptides located between the sialic acid-binding domain (SBD) and a biotin-binding moiety (BBM).

As disclosed herein, a GBS polypeptide antigen is used as a representative or exemplary polypeptide in a SBD-[Ag]n-BBM fusion protein, where the exemplary GBS polypeptide comprises at least one GBS polypeptide antigen selected from: Rib, Sip, AlpC, Alp1 or Alp3 is disclosed, e.g., See Table 2.

In some embodiments, MAPS-X immunogenic composition can comprise a SBD-[Ag]n-BBM fusion protein as disclosed in Table 2A and 2B, although such fusion proteins are representative of exemplary components of the GBS fusion proteins disclosed herein, and are not to be limited to the specific order of proteins shown, rather the proteins components of the fusion protein can be in any order.

Table 2: Exemplary SBD-[Ag]n-BBM fusion proteins in a MAPS-GBS immunogenic complex, where the fusion protein comprises at least a SBD, and at least a BBM and at least one polypeptide antigen (Ag). Shown is a SBD-[Ag]n-BBM, where n=1, 2, or 3. SBD refers to any SBD protein as disclosed herein, including but not limited to SBD1, SBD2, SBD3, SBD4, NanH, NanH2, NanH3 or VcNanH, or having an amino acid sequence with at least 80%, or at least 85%, or at least 90%, or at least 95% sequence identity to any of SEQ ID NOs: 3-12 disclosed herein. The arrangement of the proteins is illustrative and it is envisioned that any order of the proteins in the GBS fusion protein is encompassed.

| SBD-[Ag]n-BBM fusion | SBD-[Ag]n-BBM fusion | SBD-[Ag]n-BBM fusion |
|---|---|---|
| proteins: SBD-Rhavi fusion | proteins: SBD-Rhavi fusion | proteins: SBD-Rhavi fusion |
| protein, where n = 1, and each | protein, where n = 2, and each | protein, where n = 3, and each |
| are a different antigen | are a different antigen | are a 3 different antigen |
| Rhavi-[Ag1]-SBD | Rhavi-[Ag1]-[Ag2]-SBD | Rhavi-[Ag1]-[Ag2]-[Ag3]-SBD |
| Rhavi-[Ag2]-SBD | Rhavi-[Ag1]-[Ag3]-SBD | Rhavi-[Ag1]-[Ag2]-[Ag4]-SBD |
| Rhavi-[Ag3]-SBD | Rhavi-[Ag1]-[Ag4]-SBD | Rhavi-[Ag1]-[Ag3]-[Ag4]-SBD |
| Rhavi-[Ag4]-SBD | Rhavi-[Ag2]-[Ag3]-SBD | Rhavi-[Ag2]-[Ag3]-[Ag4]-SBD |
| | Rhavi-[Ag2]-[Ag4]-SBD | |
| | Rhavi-[Ag3]-[Ag4]-SBD | |

It is envisioned a MAPS-X immunogenic composition can comprise a SBD-[Ag]n-BBM fusion protein where the antigen polypeptide antigen, e.g., Ag1, Ag2, Ag3 or Ag4 as listed in Table 2 is readily be substituted by one of ordinary skill in the art for any antigenic polypeptide of interest, e.g., but not limited to any one or more of: SP0785, SP1500, SP0435 and Ply, or CP-1 as disclosed in International Applications: WO/2020/056127, WO/2020/056202, WO/2014/124228, and WO/2023/039223, and in US patents or patent application U.S. Ser. No. 11/560,410B2 and/or any polypeptide antigen as disclosed in any of: WO/2018/183475, WO/2018/217564, WO/2023/102359, WO/2023/102359A9, WO/2012/155007, WO/2023/192997A2WO/2013/134656, WO/2023/039108, WO/2012/155053, WO/2023/172741A2, WO/2014/124228, WO/2020/056127, WO/2017/192801, WO/2020/056202, WO/2018/237221, WO/2023/039223, U.S. Ser. No. 11/305,001, US20220362367, US20210008192, US20200121777, US20140154287, U.S. Ser. No. 10/766,932, US20210332090, US20230233667, U.S. Ser. No. 11/560,410, U.S. Ser. No. 10/611,805, US20160090404, U.S. Ser. No. 10/017,548, U.S. Pat. No. 9,499,593, US20140154286, US20200407404, US20190119335, US20150374811, U.S. Ser. No. 11/576,958, US20230081705, US20230089151, US20200087361, US20190119332, U.S. Ser. No. 11/013,793, U.S. Ser. No. 11/701,416, US20210346487, US20200222522, U.S. Ser. No. 11/612,647, US20220072118, US20230091255 US20150374811A1, each of which are incorporated herein in their entirety by reference.

In some embodiments, a polypeptide antigen in a SBD-[Ag]n-BBM fusion protein for use in a MAPS-X immunogenic complex can be selected from any one or more of:
(a) a S. aureus polypeptide antigen, selected from any of: alpha hemolysin (Hla), Clumping factor A (ClfA), Clumping factor B (ClfB), serine-aspirate repeat protein D (SdrD), serine-aspirate repeat protein E (SdrE), Iron regulator surface protein A (IsdA), Iron regulator surface protein B (IsdB), Leukotoxin D (LukD), or Leukotoxin E (LukE) as disclosed in WO/2018/183475A1, or an antigenic fragment thereof, or a polypeptide antigen selected from SA1720 (B1), SA1739 (B2), SA1890 (B3), SA0103 (T1), SA0377 (T2), SA0693 (T3) or SA2105 (T4) comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO: 50, 51, 52, 53, 54, 55 or 56 as disclosed in WO/2023/172741A2 or an antigenic fragment thereof, or an alpha-hemolysin (Hla) polypeptide, as disclosed in U.S. Pat. No. 11,560,410, which is incorporated herein in its entirety by reference;
(b) a Streptococcus pneumoniae polypeptide antigen, selected from any one or more of: a polypeptide comprising, or consisting of any of the polypeptide comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NOs: 1-76 or SEQ ID NO: 153-234, as disclosed in WO/2014/124228, or a polypeptide antigen selected from SP1500 or SP0785, as disclosed in WO/2020/056127, or an antigenic fragment thereof, or an antigenic fragment thereof, or an antigenic peptide comprises an amino acid sequence at least 90%, at least 91%, at least 92%, at least 93%, at least 94%, at least 95%, at least 96%, at least 97%, at least 98%, at least 99%, at least 99.5%, or 100% identical to any SEQ ID NO: 7 or SEQ ID NO: 4 as disclosed in WO/2020/056127, or an antigenic fragment thereof, or a non-hemolytic pneumolysin (Ply) polypeptide or SP0435, or an antigenic polypeptide thereof, as disclosed in WO/2023/039223, or, or an antigenic peptide comprises an amino acid sequence at least 90%, at least 91%, at least 92%, at least 93%, at least 94%, at least 95%, at least 96%, at least 97%, at least 98%, at least 99%, at least 99.5%, or 100% identical to any SEQ ID NO: 6 or SEQ ID NO: 8 as disclosed WO/2023/039223, which is incorporated herein in its entirety by reference;
(c) a Mycobacterium tuberculosis polypeptide antigen, selected from any one or more of: MPT51, ESAT6, CFPIO, MPT64, MPT83, TB9.8, TB 10.4, PPE41, and PE25 as disclosed in WO/2018/217564,
(d) a Salmonella polypeptide antigen, selected from any one or more of: SseB (Salmonella) or a Shigella polysaccharide selected from or IpaB (Shigella) as disclosed in WO/2023/102359, which is incorporated herein in its entirety by reference;
(e) a SARS-coV2 polypeptide antigen, selected from any one or more of: a Spike (S) polypeptide antigen or antigenic fragment thereof, an Envelope (E) polypeptide antigen or antigenic fragment thereof, a Membrane (M) polypeptide antigen or antigenic fragment thereof, or a Nucleocapsid (N) polypeptide or antigenic fragment thereof, e.g., a polypeptide antigen that comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, 18, 21, 24, 27, 30, 33, 36, 39 or 42 as disclosed in WO/2023/039108A1, which is incorporated herein in its entirety by reference, or an antigenic fragment thereof,
(e) a Klebsiella pneumoniae polypeptide antigen, selected from any one or more of: a polypeptide comprising, or consisting of any of the polypeptide selected from any of: FlaBD2, PcrV or a MrkA, as disclosed in U.S. Pat. No. 11,612,647, which is incorporated herein in its entirety by reference;
(f) a Pseudomonas aeruginosa polypeptide antigen, selected from any one or more of: a polypeptide comprising, or consisting of any of the polypeptide selected from any of: PcrV, PopB, PcrH.t

In some embodiments, a fusion protein useful in the MAPS-X immunogenic complex is selected from any of: SBD-[Ag1]-BBM fusion protein, SBD-[Ag1-Ag2]-BBM fusion protein, BBM-[Ag1]-SBD fusion protein, BBM-[Ag1-Ag2]-SBD fusion protein, or variations thereof, where Ag1 and Ag2 represent distinct polypeptide antigens.

In some embodiments, the MAPS-X complex comprises a bifunctional SBD-BBM fusion protein as disclosed herein, and further comprises a non bifunctional fusion protein, e.g., selected from a SBD-[Ag]n fusion protein or a BBM-[Ag]n. The antigens for a SBD-[Ag]n fusion protein or a BBM-[Ag]n fusion protein can be the same, or different to that of a SBD-[Ag]n-BBM fusion protein, as disclosed herein.

### IV. Antigenic Polysaccharides for Use in the MAPS-X

One component of a MAPS-X immunogenic complex as disclosed herein is an antigenic or immunogenic polysaccharide (PS), and can optionally, comprise additional elements that do not negatively impact the antigenic polysaccharide's function of (i) inducing an immune response to the polysaccharide and (ii) presenting at least one polypeptide antigen(s) to the immune system in immunogenic fashion. In some embodiments, the immunogenic polysaccharide is a synthetic polysaccharide.

It is envisioned that the polysaccharide used in the MAPS-X composition is immunogenic, that is, it helps induce a specific immune response, and herein is referred to as an “immunogenic polysaccharide” or “antigenic polysaccharide”. The specific immune response recognizes the particular immunogenic PS and provides a response to the immunogenic complex, and as explained herein, the response includes both a humoral and cell-mediated response.

As used herein, the term “saccharide” refers to a single sugar moiety or monosaccharide unit as well as combinations of two or more single sugar moieties or monosaccharide units covalently linked to form disaccharides, oligosaccharides, and polysaccharides. The term “saccharide” may be used interchangeably with the term “carbohydrate.” The polysaccharide may be linear or branched.

A “monosaccharide” as used herein refers to a single sugar residue in an oligosaccharide. The term “disaccharide” as used herein refers to a polysaccharide composed of two monosaccharide units or moieties linked together by a glycosidic bond. In one embodiment, the polysaccharide is an oligosaccharide (OS). An “oligosaccharide” as used herein refers to a compound containing two or more monosaccharide units or moieties. Within the context of an oligosaccharide, an individual monomer unit or moiety is a monosaccharide which is, or can be, bound through a hydroxyl group to another monosaccharide unit or moiety. Oligosaccharides can be prepared by either chemical synthesis from protected single residue sugars or by chemical degradation of biologically produced polysaccharides. Alternatively, oligosaccharides may be prepared by in vitro enzymatic methods.

In a preferred embodiment, the polysaccharide of a MAPS-X immunogenic complex is a polysaccharide (PS), which refers to a linear or branched polymer of at least 5 monosaccharide units or moieties. For clarity, a larger number of repeating units, wherein n is greater than about 5, such as greater than about 10, will be referred to herein as a polysaccharide.

In one embodiment, the polysaccharide is a cell surface polysaccharide. A cell surface polysaccharide refers to a polysaccharide having at least a portion located on the outermost bacterial cell membrane or bacterial cell surface, including the peptidoglycan layer, cell wall, and capsule. Typically, a cell surface polysaccharide is associated with inducing an immune response in vivo. A cell surface polysaccharide may be a “cell wall polysaccharide” or a “capsular polysaccharide.” A cell wall polysaccharide typically forms a discontinuous layer on the bacterial surface.

In one embodiment, the polysaccharide is a capsular polysaccharide. A capsular polysaccharide refers to a glycopolymer that includes repeating units of one or more monosaccharides joined by glycosidic linkages. A capsular polysaccharide typically forms a capsule-like layer around a bacterial cell. “Capsular polysaccharide” or “capsule polysaccharide” refers to the polysaccharide capsule that is external to the cell wall of most bacterial isolates.

In some embodiments, the immunogenic polysaccharide is a naturally occurring polysaccharide, e.g., a polysaccharide derived or purified from a pathogen, e.g., including, but not limited to, bacterial cells, and can be, for example, a capsular or noncapsular PS. In some embodiments, the immunogenic polysaccharide is derived or purified from eukaryotic cells, e.g., fungi, insect or plant cells. In yet other embodiments, the immunogenic polysaccharide is derived from mammalian cells, such as virus-infected cells or cancer cells. In general, such immunogenic polysaccharides are well known in the art and are encompassed for use in the MAPS-X immunogenic complex as disclosed herein.

In some embodiments, one or more immunogenic biotinylated polysaccharide of the MAPS-X immunogenic complex does not comprise a sialic acid. In some embodiments, a biotinylated immunogenic polysaccharide that does not natively comprise a sialic acid has a general negative polarity. In some embodiments, a biotinylated immunogenic polysaccharide that does not natively comprise a sialic acid can be sialylated, e.g., have one or more sialic acid moieties added.

In some embodiments, a MAPS-X immunogenic complex described herein includes one or more biotinylated immunogenic polysaccharide is selected from a polysaccharide from the group consisting of: S. aureus, Vi polysaccharide, pneumococcal capsular polysaccharides, pneumococcal cell wall polysaccharide, Haemophilus influenzae Type b polysaccharide, Meningococcal polysaccharide, 0-antigens from Gram-negative bacteria and other bacterial capsular or cell wall polysaccharides. In some embodiments, a MAPS-X composition as disclosed herein comprises an immunogenic polysaccharide selected from type 1 capsular polysaccharide (CP1) of Streptococcus pneumoniae, type 5 capsular polysaccharide (CP5) of S. aureus or type 8 capsular polysaccharide (CP8) of S. aureus.

Staphylococcal microorganisms capable of causing invasive disease generally also are capable of producing a capsule polysaccharide (CP) that encapsulates the bacterium and enhances its resistance to clearance by the host innate immune system. The CP serves to cloak the bacterial cell in a protective capsule that renders the bacteria resistant to phagocytosis and intracellular killing. Bacteria lacking a capsule are more susceptible to phagocytosis. Capsular polysaccharides are frequently an important virulence factor for many bacterial pathogens, including Haemophilus influenzae, Streptococcus pneumoniae and Group B streptococci.

In some embodiments, an immunogenic polysaccharide for use in the MAPS-X complex as disclosed herein can comprises N. meningitidis capsular polysaccharides from at least one, two, three or four of the serogroups A, C, W, W135, or Y. In some embodiments, an immunogenic polysaccharide for use in the MAPS-X complex as disclosed herein is selected from the group consisting of: Salmonella typhi Vi capsular polysaccharide, pneumococcal capsular polysaccharides, pneumococcal cell wall polysaccharide, Haemophilus influenzae Type b (Hibb) capsular polysaccharide, Haemophili polysaccharide, Meningococcal polysaccharide, polysaccharides or oligosaccharides from Gram-positive bacteria (e.g., Staphylococcus aureus capsular polysaccharide, Bacillus anthracis polysaccharide), Streptococcus polysaccharides (e.g., Gp A and Gp B), Pseudomonas polysaccharide, fungal polysaccharides (e.g., cryptococcus polysaccharides), viral polysaccharides (e.g., glycoprotein) and other bacterial capsular or cell wall polysaccharides. In some embodiments, an immunogenic polysaccharide is selected from any of the following, dextran, Vi polysaccharide of Salmonella typhi, pneumococcal capsular polysaccharide, pneumococcal cell wall polysaccharide (CWPS), meningococcal polysaccharide, Haemophilus influenzae type b polysaccharide, or any another polysaccharide of viral, prokaryotic, or eukaryotic origin.

It is important that a Capsule Polysaccharide (CP) used in the MAPS-X immunogenic complex as disclosed herein is immunogenic. The molecular weight of a capsule polysaccharides is an important consideration, as a high molecular weight capsule polysaccharide can induce certain antibody immune responses due to a higher valency of the epitopes present on the antigenic surface. In some embodiments, a CP8 or CP5 used in a MAPS-X immunogenic complex as disclosed herein is a high molecular weight capsule polysaccharide type 5 (CP5) and type 8 (CP8) from S. aureus or a CP1 of Streptococcus pneumonia, CP1) of Streptococcus pneumoniae, however, it is envisioned that any high molecular weight capsule polysaccharide from a pathogen can be used as an antigenic polysaccharide in the MAPS-X immunogenic complex as disclosed herein.

In some embodiments, the immunogenic polysaccharide for use in MAPS-X complex as disclosed herein is a branched polymer. In some embodiments, an immunogenic polysaccharide for use in the MAPS-X complex as disclosed herein is a single chain polymer.

In some embodiments, an immunogenic polysaccharide for use in the MAPS-X complex as disclosed herein that can serve as a backbone for one or more protein antigen types are exemplified in Table 3:

TABLE 3
Example immunogenic polysaccharides for the MAPS-X
backbone and associated example antigens

| | | Protein Antigens | |
|---|---|---|---|
| Polysaccharide | | Number of antigens | Antigen origins |
| Dextran | D90 (60-90KD) | Two | Staphylococcus aureus |
| | D150 (150 KD) | Three | Staphylococcus aureus |
| | D270 (270 KD) | Three | Staphylococcus aureus |
| | D500 (425-575 | two; three; six | Staphylococcus aureus |
| | KD) | | |
| Pneumococcal | Serotype 1 | one, two, three, five | pneumococcus, |
| capsular | | | tuberculosis, |
| polysaccharide | | | Staphylococcus aureus |
| | Serotype 3 | Five | pneumococcus, |
| | | | tuberculosis, |
| | | | Staphylococcus aureus |
| | Serotype 5 | one; two; three; five | pneumococcus, |
| | | | tuberculosis, |
| | | | Staphylococcus aureus |
| | Serotype 6B | Two | Pneumococcus, |
| | | | Staphylococcus aureus |
| | Serotype 7 | Three | Pneumococcus, |
| | | | Staphylococcus aureus |
| | Serotype 14 | one; two; three; five | pneumococcus, tuberculosis |
| | Serotype 19 | Three | Pneumococcus, |
| | | | Staphylococcus aureus |
| Staphylococcus aureus | Serotype 5 | one; two; three; five, | Staphylococcus aureus |
| | | six | |
| | Serotype 8 | one; two; three; five, | Staphylococcus aureus |
| | | six | |
| | PNAG | one; two; three; five, | Staphylococcus aureus |
| | | six | |
| | S. aureus 336 | one; two; three; five, | Staphylococcus aureus |
| | antigen | six | |

| Pneumococcal cell wall polysaccharide | Five | Pneumococcus, |
|---|---|---|
| | | Staphylococcus aureus |
| S. typhi Vi polysaccharide | Five | Pneumococcus, |

Staphylococcus aureus

In some embodiments, the immunogenic polysaccharide for use in the MAPS-X complex as disclosed herein comprises at least 10 carbohydrate repeating units, or at least 20, or at least 50, or at least 75, or at least 100, or at least 150, or at least 200, or at least 250, or at least 300, or at least 350, or at least 400, or at least 450, or at least 500, or more than 500 repeating units, inclusive.

In one aspect of the invention, the immunogenic polysaccharide (PS) for use in the MAPS-X complex as disclosed herein can have a molecular mass of <500 kDa or >500 kDa. In another aspect of the invention, the PS has a molecular mass of <70 kDa. In some embodiments, an immunogenic polysaccharide for use in the MAPS-X complex as disclosed herein is a large molecular weight polymer, e.g., a polymer can be of an average molecular weight of between about 425-500 kDa, inclusive, for example, at least 300 kDa, or at least 350 kDa, or at least 400 kDa, or at least 425 kDa, or at least 450 kDa, or at least 500 kDa or greater than 500 kDa, inclusive, but typically less than 500 kDa. In some embodiments, an immunogenic polysaccharide for use in the MAPS-X complex as disclosed herein can be a small molecular weight polymer, e.g., a polymer can be of an average molecular weight of between about 60 kDA to about 90 kDa, for example, at least 50 kDa, or at least 60 kDa, or at least 70 kDa, or at least 80 kDa, or at least 90 kDa, or at least 100 kDa, or greater than 100 kDa, inclusive, but generally less than about 120 kDa.

In some embodiments, the immunogenic polysaccharide for use in the MAPS-X complex as disclosed herein is harvested and purified from a natural source; and in other embodiments, the polysaccharide is synthetic. Methods to produce synthetic polymers, including synthetic polysaccharides, are known to persons of ordinary skill and are encompassed in the compositions and methods as disclosed herein.

In some embodiments, a type 5 and/or type 8 capsular polysaccharide or oligosaccharide included in a MAPS-X immunogenic compositions as disclosed herein has a molecular weight of between 20 kDa and 1000 kDa. In some embodiments, the type 5 and/or type 8 and/or type 1 capsular polysaccharide or oligosaccharide of a MAPS-X immunogenic compositions as disclosed herein has a molecular weight of between 200 kDa and 5000 kDa, or a molecular weight range of between 70 kDa and 300 kDa, or a molecular weight range of between 500 kDa and 2500 kDa.

High molecular weight capsular polysaccharides are able to induce certain antibody immune responses due to a higher valence of the epitopes present on the antigenic surface. The isolation of “high molecular weight capsular polysaccharides” is contemplated for use in the compositions and methods of the present invention. In some embodiments, high molecular weight serotype 5 or 8 capsular polysaccharide can be isolated and purified ranging from 20 kDa to 1000 kDa in molecular weight. In one embodiment, high molecular weight serotype 5 or 8 capsular polysaccharide can be isolated and purified ranging from 50 kDa to 700 kDa in molecular weight, or ranging from 50 kDa to 300 kDa in molecular weight, or ranging from 70 kDa to 300 kDa, or ranging from 90 kDa to 250 kDa, or ranging from 90 kDa to 150 kDa in molecular weight, or ranging from 90 kDa to 120 kDa in molecular weight, or ranging from 80 kDa to 120 kDa in molecular weight. In some embodiments, a type 1, and/or type 5 and/or type 8 capsular polysaccharide or oligosaccharide included in a MAPS-X immunogenic compositions as disclosed herein has a high molecular weight of any of 70 kDa to 100 kDa in molecular weight; 70 kDa to 110 kDa in molecular weight; 70 kDa to 120 kDa in molecular weight; 70 kDa to 130 kDa in molecular weight; 70 kDa to 140 kDa in molecular weight; 70 kDa to 150 kDa in molecular weight; 70 kDa to 160 kDa in molecular weight; 80 kDa to 110 kDa in molecular weight; 80 kDa to 120 kDa in molecular weight; 80 kDa to 130 kDa in molecular weight; 80 kDa to 140 kDa in molecular weight; 80 kDa to 150 kDa in molecular weight; 80 kDa to 160 kDa in molecular weight; 90 kDa to 110 kDa in molecular weight; 90 kDa to 120 kDa in molecular weight; 90 kDa to 130 kDa in molecular weight; 90 kDa to 140 kDa in molecular weight; 90 kDa to 150 kDa in molecular weight; 90 kDa to 160 kDa in molecular weight; 100 kDa to 120 kDa in molecular weight; 100 kDa to 130 kDa in molecular weight; 100 kDa to 140 kDa in molecular weight; 100 kDa to 150 kDa in molecular weight; 100 kDa to 160 kDa in molecular weight; and similar desired molecular weight ranges. Any whole number integer within any of the above ranges is contemplated as an embodiment of the invention.

In one embodiment, the MAPS-X complex has a molecular weight of between about 50 kDa and about 5000 kDa in molecular weight. In one embodiment, the MAPS-X complex has a molecular weight of between about 200 kDa and about 5000 kDa in molecular weight. In one embodiment, the immunogenic MAPS-X complex has a molecular weight of between about 500 kDa and about 2500 kDa. In one embodiment, the immunogenic MAPS-X complex has a molecular weight of between about 500 kDa and about 2500 kDa. In one embodiment, the immunogenic MAPS-X complex has a molecular weight of between about 600 kDa and about 2800 kDa. In one embodiment, the immunogenic MAPS-X complex has a molecular weight of between about 700 kDa and about 2700 kDa. In one embodiment, the immunogenic MAPS-X complex has a molecular weight of between about 1000 kDa and about 2000 kDa; between about 1800 kDa and about 2500 kDa; between about 1100 kDa and about 2200 kDa; between about 1900 kDa and about 2700 kDa; between about 1200 kDa and about 2400 kDa; between about 1700 kDa and about 2600 kDa; between about 1300 kDa and about 2600 kDa; between about 1600 kDa and about 3000 kDa. Any whole number integer within any of the above ranges is contemplated as an embodiment of the MAPS-X immunogenic complex as disclosed herein.

In one embodiment, the serotype 5 or 8 capsular polysaccharide has a degree of O-acetylation between 10-100%. In one embodiment, the degree of O-acetylation is between 50-100%. In one embodiment, the degree of O-acetylation is between 75-100%.

In some embodiments, an immunogenic polysaccharide for use in the MAPS-X complex as disclosed herein can comprise additional polymers, for example, polyethylene glycol-based polymers, poly(ortho ester) polymers, polyacryl carriers, PLGA, polyethylenimine (PEI), polyamidoamine (PAMAM) dendrimers, R-amino ester polymers, polyphosphoester (PPE), liposomes, polymerosomes, nucleic acids, phosphorothioated oligonucleotides, chitosan, silk, polymeric micelles, protein polymers, virus particles, virus-like-particles (VLPs) or other micro-particles. See, e.g., El-Sayed et al., Smart Polymer Carriers for Enhanced Intracellular Delivery of Therapeutic Molecules, 5 Exp. Op. Biol. Therapy, 23 (2005). Biocompatible polymers developed for nucleic acid delivery may be adapted for use as a backbone herein. See, e.g., BIOCOMPATIBLE POL. NUCL. ACID. DELIV. (Domb et al., eds., John Wiley & Sons, Inc. Hoboken, NJ, 2011).

For example, VLPs resemble viruses, but are non-infectious because they do not contain any viral genetic material. The expression, including recombinant expression, of viral structural proteins, such as envelope or capsid components, can result in the self-assembly of VLPs. VLPs have been produced from components of a wide variety of virus families including Parvoviridae (e.g., adeno-associated virus), Retroviridae (e.g., HIV), and Flaviviridae (e.g., Hepatitis B or C viruses). VLPs can be produced in a variety of cell culture systems including mammalian cell lines, insect cell lines, yeast, and plant cells. Recombinant VLPs are particularly advantageous because the viral component can be fused to recombinant antigens as described herein.

### (a) Sialic Acid Polysaccharides:

In some embodiments, an antigenic polysaccharide in a MAPS-X immunogenic complex comprises a “sialic acid”. In some embodiments, PS1 or PS2, or both comprises a sialic acid. In some embodiments, neither PS1 or PS2 comprise a sialic acid. The term “sialic acid” as used herein, embraces all forms of N- or O-substituted neuraminic acid and includes all synthetic, naturally occurring and/or modified forms thereof. Sialic acids may be found as components of cell surface molecules, glycoproteins and glycolipids. Most often, sialic acids are present at the end (terminal regions) of sugar chains connected to cell membranes and/or proteins. The sialic acid family encompasses a number (approximately 50) of derivatives that may result from acetylation, glycosylation, lactonization and methylation at C4, C5, C7, C8 and C9. All such derivatives are to be embraced by the term “sialic acid”.

Furthermore, sialic acids are found linked a(2,3) or a(2,6) to Gal and GalNAc or a(2,8) or a(2,9) to another sialic acid. Accordingly, it is important to understand that while the term “sialic acid” is used throughout this specification, it encompasses all derivatives, analogues or variants (either naturally occurring or synthetically generated) thereof as well as monomers, dimers, trimers, oligomers, polymers or concatemers comprising the same.

In some embodiments, the immunogenic polysaccharide is sialylated polysaccharide, e.g., comprises at least one sialic acid moiety, as disclosed herein. In some embodiments, the native level of sialic acid on a polysaccharide is modified, e.g., the percentage increased. In one embodiment, a capsular polysaccharides of a MAPS-X complex can comprise their natural sialic acid level, such as about 100% or greater than about 95%. In another embodiment, the capsular polysaccharides may be desialylated up to about 40% (sialylation level greater than about 60%), such as up to about 35% (sialylation level greater than about 65%), up to about 30% (sialylation level greater than about 70%), up to about 25% (sialylation level greater than about 75%), up to about 20% (sialylation level greater than about 80%), up to about 15% (sialylation level greater than about 85%), up to about 10% (sialylation level greater than about 90%), and up to about 5% (sialylation level greater than about 95%).

It should be noted that 100% sialic acid level corresponds to about 1.0 mM sialic acid per mM of polysaccharide. Therefore, the capsular polysaccharides may have about 1.0 mM sialic acid per mM of polysaccharide, such as at least about 0.95 mM sialic acid per mM of polysaccharide. In a further embodiment, the capsular polysaccharide may have at least about 0.6 mM sialic acid per mM of polysaccharide, such as at least about 0.65 mM sialic acid per mM of polysaccharide, at least about 0.7 mM sialic acid per mM of polysaccharide, at least about 0.75 mM sialic acid per mM of polysaccharide, at least about 0.8 mM sialic acid per mM of polysaccharide, at least about 0.85 mM sialic acid per mM of polysaccharide, at least about 0.9 mM sialic acid per mM of polysaccharide, or at least about 0.95 mM sialic acid per mM of polysaccharide.

The terminal sialic residues of some capsular polysaccharide (CP) serotypes are partially 0-acetylated (OAc) (Lewis, A. L., et al., Proceedings of the National Academy of Sciences USA, 101 (30): 1 1 123-8 (2004)). GBS Serotypes 1b, III, IV, V, VI, and IX are partially O-acetylated (up to −40%), whereas serotypes 1a, II, and VII have little or no O-acetylation (less than about 5%) (Lewis 2004). In one embodiment of the invention, the capsular polysaccharides comprise their natural 0-acetylation level (about 0% to about 40%). In another embodiment, the capsular polysaccharides may be de-O-acetylated (less than about 5%). The degree of O-acetylation of the polysaccharide or oligosaccharide can be determined by any method known in the art, for example, by proton NMR (Lemercinier, X., et al., Carbohydrate Research, 296:83-96 (1996); Jones, C, et al., Journal of Pharmaceutical and Biomedical Analysis, 30: 1233-1247 (2002); Int′l Patent Appl. Pub. Nos. WO 2005/033148 and WO 00/56357). Another commonly used method is described by Hestrin, S., J. Biol. Chem., 180:249-261 (1949).

It should also be noted that 100% O-acetate corresponds to about 1.0 mM O-acetate per mM of saccharide repeating unit. Accordingly, partially 0-acetylated polysaccharides comprise at least about 0.1, 0.2, 0.3, 0.35 or about 0.4 mM O-acetate per mM saccharide repeating unit. A de-O-acetylated polysaccharide comprises less than about 0.01, 0.02, 0.03, 0.04, or 0.05 mM O-acetate per mM saccharide repeating unit.

(b) Poly N-Acetylated Glucosamine (PNAG) polysaccharides: In some embodiments, an antigenic polysaccharide in a MAPS-X immunogenic complex comprises PNAG. PNAG is a polysaccharide intercellular adhesion and is composed of a polymer of β-(1→6)-linked glucosamine, optionally substituted with N-acetyl and/or O-succinyl constituents. This polysaccharide is present in both S. aureus and S. epidermidis and can be isolated from either source (Joyce et al 2003, Carbohydrate Research 338; 903; Maira-Litran et al 2002, Infect. Imun. 70; 4433). For example, PNAG may be isolated from S. aureus strain MN8m (WO 04/43407). The preparation of dPNAG is described in WO 04/43405, which is incorporated herein in its entirety by reference. The term PNAG as used herein also encompasses a polysaccharide previously known as poly-N-succinyl-β-(1→6)-glucosamine (PNSG) (Maira-Litran et al 2002, Infect. Imun. 70; 4433).

PNAG may be of different sizes varying from over 400 kDa to between 75 and 400 kDa to between 10 and 75 kDa to oligosaccharides composed of up to 30 repeat units (of (0-(1→6)-linked glucosamine, optionally substituted with N-acetyl and O-succinyl constituents). Any size of PNAG polysaccharide or oligosaccharide may be used in an MAPS-X immunogenic complex of the invention, for example a size of over 40 kDa can be used. Sizing may be achieved by any method known in the art, for instance by microfluidisation, ultrasonic irradiation or by chemical cleavage (WO 03/53462, EP497524, EP497525). Size ranges of PNAG are for example 40-400 kDa, 50-350 kDa, 40-300 kDa, 60-300 kDa, 50-250 kDa and 60-200 kDa. PNAG can have different degree of acetylation due to substitution on the amino groups by acetate. PNAG produced in vitro is almost fully substituted on amino groups (95-100%). Alternatively, a deacetylated PNAG can be used having less than 50%, 40%, 30%, 20%, 10% or 5% N-acetylation. Use of a deacetylated PNAG allows opsonic killing of Gram positive bacteria, optionally S. aureus and/or S. epidermidis (WO 04/43405). In an embodiment, the PNAG has a size between 40 kDa and 300 kDa and is deacetylated so that less than 50%, 40%, 30%, 20%, 10% or 5% of amino groups are N acetylated. In an embodiment, the PNAG is not O-succinylated or is O-succinylated on less than 25, 20, 15, 10, 5, 2, 1 or 0.1% of residues. The term deacetylated PNAG (dPNAG) refers to a PNAG polysaccharide or oligosaccharide in which less than 50%, 40%, 30%, 20%, 10% or 5% of the amino groups are acetylated.

As used herein, the term PNAG encompasses both acetylated and deacetylated forms of the saccharide. In an embodiment, PNAG is deacetylated to form dPNAG, by chemically treating the native polysaccharide. For example, the native PNAG is treated with a basic solution such that the pH rises to above 10. For instance, the PNAG is treated with 0.1-5M, 0.2-4M, 0.3-3M, 0.5-2M, 0.75-1.5M or 1M NaOH, KOH or NH₄OH. Treatment is for at least 10 or 30 minutes, or 1, 2, 3, 4, 5, 10, 15 or 20 hours at a temperature of 20-100, 25-80, 30-60 or 30-50 or 3545° C. dPNAG may be prepared as described in WO 04/43405.

In one embodiment, the MAPS-X immunogenic conjugate generates an antibody that is functional as measured by killing bacteria in either an animal efficacy model or via an opsonophagocytic killing assay.

(c) Polysaccharides from Specific Pathogens
(i) S. pneumoniae Polysaccharides

In some embodiments an immunogenic polysaccharide for use in the MAPS-X complex as disclosed herein can be a pneumococcal polysaccharide, e.g., a capsular polysaccharide from Streptococcus pneumoniae from any of the over 93 serotypes of pneumococcus that have been identified to date, for example, including but not limited to serotypes 1, 2, 3, 4, 5, 6A, 6B, 6C, 6D, 7F, 8, 9N, 9V, 10A, 11A, 12F, 14, 15B, 17F, 18C, 19A, 19F, 20, 22F, 23F, and 33F. Additional pneumococcal serotypes may be identified and included in the present MAPS-X immunogenic composition as described herein. More than one pneumococcal polysaccharide can be included as the polysaccharide in a MAPS-X immunogenic complex as disclosed herein. In some embodiments, one species of MAPS-X in a vaccine composition comprises a pneumococcal polysaccharide from one serotype, and another species of MAPS-X in a vaccine composition as disclosed herein comprises a pneumococcal polysaccharide from a different serotype. In some embodiments, an immunogenic polysaccharide for use in the MAPS-X complex as disclosed herein is Type 1 capsular polysaccharide (CP1) from Streptococcus pneumoniae.

In some embodiments, an immunogenic polysaccharide for use in the MAPS-X complex as disclosed herein comprises a polysaccharide of Streptococcus pneumoniae having a serotype selected from one or more of 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48, as disclosed in U.S. Pat. No. 11,013,793, which is incorporated herein in its entirety by reference. In some embodiments, the immunogenic polysaccharide is a polysaccharide antigen of Streptococcus pneumoniae which comprises a polysaccharide of Streptococcus pneumoniae having a serotype selected from one or more of 1, 2, 3, 4, 5, 6A, 6B, 7F, 8, 9N, 9V, 10A, 11A, 12F, 14, 15B, 17F, 18C, 19A, 19F, 20B, 22F, 23F, and 33F, as disclosed in U.S. Pat. No. 11,013,793, which is incorporated herein in its entirety by reference.

In some embodiments, a vaccine as disclosed herein comprises a plurality of species of MAPS-X immunogenic complexes, where each species of MAPS-X complex comprises a polysaccharide antigen from a distinct Streptococcus pneumoniae serotype, where the Streptococcus pneumoniae serotypes are selected from one or more of Streptococcus pneumoniae serotypes: 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.

### (ii) S. Aureus Polysaccharides

In some embodiments, an immunogenic polysaccharide for use in a MAPS-X immunogenic complex as disclosed herein is a polysaccharide or oligosaccharide from Gram-positive bacteria, for example, a Staphylococcus aureus capsular polysaccharide.

Type 5 and Type 8 Polysaccharides from S. aureus

Most strains of S. aureus that cause infection in man contain either Type 5 or Type 8 polysaccharides. Approximately 60% of human strains are Type 8 and approximately 30% are Type 5. The structures of Type 5 and Type 8 capsular polysaccharide antigens are described in Moreau et al Carbohydrate Res. 201; 285 (1990) and Fournier et al Infect. Immun. 45; 87 (1984). Both have FucNAcp in their repeat unit as well as ManNAcA which can be used to introduce a sulfhydryl group.

Recently (Jones Carbohydrate Research 340, 1097-1106 (2005)) NMR spectroscopy revised the structures of the capsular polysaccharides to:

Type 5→4)-β-D-ManNAcA-(1→4)-α-L-FucNAc(3OAc)-(1→3)-β-D-FucNAc-(1→Type 8→3)-β-D-ManNAcA(4OAc)-(1→3)-α-L-FucNAc(1→3)-α-D-FucNAc(1→

Polysaccharides may be extracted from the appropriate strain of S. aureus using methods well known to the skilled man, for instance as described in U.S. Pat. No. 6,294,177 or Infection and Immunity (1990) 58(7); 2367, Fournier et al. (1984), supra; Fournier et al. (1987) Ann. Inst. Pasteur/Microbiol. 138:561-567; US Patent Application Publication No. 2007/0141077; and Int′l Patent Application Publication No. WO 00/56357; each of which is incorporated herein by reference as if set forth in its entirety). For example, ATCC 12902 is a Type 5 S. aureus strain and ATCC 12605 is a Type 8 S. aureus strain. In addition, they can be produced using synthetic protocols. Moreover, serotype 5 or 8 capsular polysaccharide can be recombinant produced using genetic engineering procedures also known to one of ordinary skill in the art (see, Sau et al. (1997) Microbiology 143:2395-2405; and U.S. Pat. No. 6,027,925; each of which is incorporated herein by reference as if set forth in its entirety).

One S. aureus strain that can be used to obtain isolated serotype 8 capsular polysaccharide is S. aureus R2 PFESA0286. This strain was selected by flow cytometry with rabbit anti-serotype 8 polysaccharide antibodies after cultivation of S. aureus PFESA0286 (American Type Culture Collection; Manassas, Va.: ATCC Accession No. 495:25) in Modified Frantz Broth. Two populations, R1 and R2, were observed during flow cytometry. R1 and R2 were purified and re-cultured. R2 yielded a serotype 8 capsular polysaccharide. Flow cytometric analysis showed a homogenous fluorescence intensity. As such, R2 was selected for serotype 8 capsular polysaccharide production. One S. aureus strain that can be used to obtain isolated serotype 5 capsular polysaccharide is S. aureus PFESA0266. This strain produces serotype 5 capsular polysaccharide during growth, and production peaks when cells are in a stationary phase. Other S. aureus type 5 or type 8 strains can be used to make the respective polysaccharides that are obtained either from established culture collections or clinical specimens. In some embodiments, a Becker or Newman S. aureus strain can be used to obtain isolated serotype 5 capsular polysaccharide (CP5). In some embodiments, the Newman S. aureus strain can be used to obtain isolated serotype 5 capsular polysaccharide (CP5). In some embodiments, a Becker or Newman S. aureus strain can be used to obtain isolated serotype 8 capsular polysaccharide (CP8). In some embodiments, the Becker S. aureus strain can be used to obtain isolated serotype 8 capsular polysaccharide (CP8).

Polysaccharides are of native size or alternatively may be sized, for instance by microfluidisation, ultrasonic irradiation or by chemical treatment. The invention also covers oligosaccharides derived from the type 5 and 8 polysaccharides from S. aureus.

In some embodiments, an immunogenic polysaccharide for use in the MAPS-SA complex as disclosed herein can comprises a Type 5 (CP5), or Type 8 (CP8) capsular polysaccharides (CP), or any of the polysaccharides or oligosaccharides or lipopolysaccharides from Staphylococcus aureus. In some embodiments, an immunogenic polysaccharide for use in the MAPS-SA complex as disclosed herein can comprises a capsular polysaccharide from a non-typeable (NT) SA strain, e.g., a cell wall surface antigen 336 (Type 336) or a polyribitol phosphate N-acetylglucosamine, which resembles cell wall teichoic acid. Type 336 isolates do not express capsule but do express cell surface polysaccharide or the 336 polysaccharide (336PS), which resembles S. aureus cell wall teichoic acid (Ma, J., et al., 2004. Evaluation of serotypes of Staphylococcus aureus strains used in the production of a bovine mastitis bacterin. J. Dairy. Sci. 87:178-182 14, 17; O'Brien, et al., 2000. Production of antibodies to Staphylococcus aureus serotypes 5, 8, and 336 using poly(dl-lactide-co-glycolide) microspheres. J. Dairy Sci. 83:1758-1766).

In some embodiments, an immunogenic polysaccharide for use in the MAPS-X complex as disclosed herein can comprises a capsular polysaccharide (CP) from a methicillin-resistant S. aureus (MRSA), including hospital-acquired MRSA (HA-MRSA), or community-acquired MRSA (CA-MRSA) or any polysaccharides or oligosaccharides or lipopolysaccharides from MRSA, e.g., e.g., any one or more of a CP5, or CP8 from HA-MSSA and/or CA-MRSA. In alternative embodiments, an immunogenic polysaccharide for use in the MAPS-X complex as disclosed herein can comprises a capsular polysaccharide (CP) from a methicillin-sensitive S. aureus (MSSA), e.g., any one or more of a CP5, or CP8 from MSSA.

The association of particular capsule serotypes with disease is possible through monitoring of clinical isolates. Of the eight different serotypes of S. aureus identified (Karakawa and Vann (1982) only serotypes 1 and 2 are heavily encapsulated, and these are rarely isolated. See Capsular Polysaccharides of Staphylococcus aureus, p. 285-293, In J. B. Robbins, J. C. Hill and J. C. Sadoff (ed.), Seminars in infectious disease, vol. 4, Bacterial Vaccines. Thieme Stratton, Inc. New York). Surveys have shown that approximately 85-90% of S. aureus clinical isolates express CP5 or CP8 (Arbeit R D, et al., Diagn. Microbiol. Infect. Dis. (1984) Apr. 2(2):85-91; Karakawa W W, et al., J. Clin. Microbiol. (1985) Sep. 22(3):445-7; Essawi T, et al., Trop. Med. Int. Health. (1998) Jul. 3(7):576-83; Na′was T, et al., J. Clin. Microbiol. (1998) 36(2):414-20. Most of CP5 and CP8 non-typeable strains are genetically type 5 or type 8 containing mutations in cap5/8 locus (Cocchiaro, Gomez et al., (2006), Mol. Microbiol. February 59(3):948-960). Capsulation for some strains is lost rapidly within few passages in vitro which is due to a repressive effect of high phosphate concentration in media used in clinical diagnosis on capsule production. It was also reported that non-capsulated isolates recover capsule expression after passing through cows. See Opdebeck, J. P. et al., J. Med. Microbiol. 19:275-278 (1985). Some non-typeable strains become capsule positive under appropriate growth conditions.

CP5 and CP8 Structure: The repeat unit of both CP5 and CP8 is comprised of 2-acetamido-2-deoxy-D-mannuronic acid, 2-acetamido-2-deoxy-L-fucose and 2-acetamido-2-deoxy-D-fucose. See C. Jones et al., Carbohydr. Res. 340:1097-1106 (2005). Although CP5 and CP8 have the same sugar composition, they have been demonstrated to be immunologically distinct. They differ in glycosidic linkages and site of O-acetylation of uronic acid. Strain dependent incomplete N-acetylation of one of the FucNAc residues was observed. See Tzianabos et al., PNAS V98: 9365 (2001).

(iii) GBS Polysaccharides

In some embodiments, a MAPS-X immunogenic complex described herein includes one or more biotinylated GBS polysaccharides (PS). In some embodiments, a MAPS-X immunogenic complex as described herein comprises a biotinylated polysaccharide from GBS. In some embodiments, a MAPS-X immunogenic complex includes one or more biotinylated GBS capsular polysaccharides or biotinylated O-specific polysaccharides (OSP) from, or derived from, one or more GBS subtypes selected from group consisting of serotypes Ia, Ib, II, III, IV, V, VI, VII, VIII, and IX.

In some embodiments, a MAPS-X immunogenic complex described herein comprises a GBS polysaccharide that is >60 kDa, or >70 kDa, or >80 kDa, or >90 kDa, or >100 kDa, or >110 kDa, or >120 kDa. In some embodiments, an immunogenic complex described herein comprises an OSP polysaccharide from GBS that is between 90-110 kDa.

In some embodiments, the first (PS1), or second polysaccharide (PS2), or both is isolated from Streptococcus agalactiae. The polysaccharide may be isolated from any encapsulated strain of S. agalactiae, such as 090, A909 (ATCC Accession No. BAA-1138), 515 (ATCC Accession No. BAA-1177), B523, CJB524, MB 4052 (ATCC Accession No. 31574), H36B (ATCC Accession No. 12401), S40, S42, MB 4053 (ATCC Accession No. 31575), M709, 133, 7357, PFEGBST0267, MB 4055 (ATCC Accession No. 31576), 18RS21 (ATCC Accession No. BAA-1175), S16, S20, V8 (ATCC Accession No. 12973), DK21, DK23, UAB, 5401, PFEGBST0708, MB 4082 (ATCC Accession No. 31577), M132, 110, M781 (ATCC Accession No. BAA-22), D136C(3) (ATCC Accession No. 12403), M782, S23, 120, MB 4316 (M-732; ATCC Accession No. 31475), M132, K79, COH1 (ATCC Accession No. BAA-1176), PFEGBST0563, 3139 (ATCC Accession No. 49446), CZ-NI-016, PFEGBST0961, 1169-NT1, CJB111(ATCC Accession No. BAA-23), CJB112, 2603 V/R (ATCC Accession No. BAA-611), NCTC 10/81, CJ11, PFEGBST0837, 118754, 114852, 114862, 114866, 118775, B 4589, B 4645, SS1214, CZ-PW-119, 7271, CZ-PW-045, JM9130013, JM9130672, IT-N1-016, IT-PW-62, and IT-PW-64. Polysaccharides isolated from Streptococcus agalactiae useful in the immunogenic complexes as disclosed herein are disclosed in U.S. Pat. No. 10,226,525, which is incorporated herein in its entirety by reference.

In some embodiments, the PS1 and PS2 for each immunogenic complex of each species is from the same GBS serotype. In some embodiments, the PS1 and PS2 for each MAPS-X immunogenic complex for each species is from a different GBS serotype. For example, PS1 and PS2 for a particular species of an MAPS-X immunogenic complex can be from, e.g., a specific serotype of Group B Streptococcus (GBS) or Streptococcus agalactiae. In alternative embodiments, PS1 and PS2 for a particular species of an immunogenic complex can be from, e.g., different serotypes of Group B Streptococcus (GBS) or Streptococcus agalactiae.

Aspects of the technology described herein relate to a composition, which is a polyvalent immune composition, and comprises at least one species of MAPS-X immunogenic complex. That is, each MAPS-X species is distinct from the other MAPS-X species in the immunogenic complex. Without being limited to theory, for example, a composition can comprise, e.g., two MAPS-X immunogenic complexes as disclosed herein, where the first species of MAPS-X immunogenic complex disclosed herein comprises a PS1 and PS2 from Streptococcus agalactiae serotype Ia, and the second species of MAPS-X immunogenic complex can comprise a PS1 and PS2 from Streptococcus agalactiae serotype 1b. In such an embodiment, a composition would be considered a two valent (2V) MAPS-X immunogenic composition or vaccine. It is envisioned that a MAPS-X polyvalent immune composition as disclosed herein comprises MAPS-X immunogenic complexes that comprise polysaccharides from at least 2, or at least 3, or at least 4, or at least 5, or at least 6, or at least 7, or at least 8, or at least 9, or at least 10, or 2-5, or 5-6, or 6-7, or 7-8, or 8-9 or 9-10, or more than 10 different serotypes of GBS bacteria.

In some embodiments, as disclosed herein, the first biotinylated polysaccharide (PS1), or second biotinylated polysaccharide (PS2), or both, is from any of serotypes 1a, Ib, II, III, IV, V, VI, VII, VIII, and IX of Streptococcus agalactiae. In some embodiments, as disclosed herein, the first biotinylated polysaccharide, or second biotinylated polysaccharide, or both, is from any of serotypes 1a, Ib, II, III, IV, V or VII of Streptococcus agalactiae (7V MAPS-X). In some embodiments, as disclosed herein, the first biotinylated polysaccharide, or second biotinylated polysaccharide, or both, is from any of serotypes 1a, Ib, II, III, V or VII of Streptococcus agalactiae (6V MAPS-X).

### Methods of Isolating and Purifying Polysaccharides

In some embodiments, the disclosure provides methods of purifying one or more polysaccharides described herein from a pathogen or from cellular components of bacteria. In some embodiments, methods comprise purifying capsular polysaccharides from one or more cellular components of bacteria. In some embodiments, the cellular components include protein. In some embodiments, the cellular proteins include nucleic acid. In some embodiments, the cellular components include lipids. In some embodiments, the cellular components include polysaccharides. In some embodiments, the cellular components are part of a lysate.

In some embodiments, the polysaccharide purification processes incorporate a series of ethanol precipitations, washes of crude polysaccharide preparations with ethanol, diethyl ether, and/or acetone, and drying under vacuum to furnish purified products. In some embodiments, a phenol extraction step is incorporated for polysaccharide purifications. In some embodiments the purification process employs a CTAB (cetyltrimethyl ammonium bromide) precipitation step in addition to using ethanol and phenol precipitation steps.

### Methods of Biotinylating Polysaccharides

In some embodiments, the disclosure provides methods of biotinylating one or more polysaccharides described herein. In some embodiments, the method comprises reacting purified polysaccharides with 1-cyano-4-dimethylaminopyridinium tetrafluoroborate (CDAP) for activation of hydroxyl groups in the polysaccharides followed by the addition of amine PEG biotin under conditions that result in covalent linkage of biotin to the polysaccharides. In some embodiments, the desired level of biotinylation is achieved by varying the ratio of CDAP to polysaccharide. In some embodiments, the method comprises reacting purified polysaccharides with 1-Ethyl-3-[3-dimethylaminopropyl]carbodiimide Hydrochloride (EDC) and N-hydroxysulfosuccinimide (NHS). In some embodiments, the biotinylated polysaccharides are purified by filtration to remove process residuals such as unreacted biotin, dimethylaminopyridine, acetonitrile, cyanide and unreacted glycine. In some embodiments, the level of polysaccharide biotinylation described herein is optimized to reduce the amount of accessible biotin following MAPS complexation.

### V. Nucleic Acids Encoding the SBD-BBM Fusion Proteins or Nucleic Acid Vaccine Compositions

In some embodiments, the present disclosure provides nucleic acids, e.g., DNA, RNA, or analogs thereof, encoding one or more of the polypeptides and/or fusion proteins described herein. An underlying DNA sequence for the polypeptides described herein may be modified in ways that do not affect the sequence of the protein product, and such sequences are included in the invention. In some embodiments, a DNA sequence may be codon-optimized to improve expression in a host such as a bacterial cell line, e.g., E. coli, an insect cell line (e.g., using the baculovirus expression system), or a mammalian (e.g., human or Chinese Hamster Ovary) cell line.

In some embodiments, the present disclosure provides nucleic acids, e.g., DNA, RNA, or analogs thereof, that are at least 70%, 80%, 85%, 90%, 95%, 97%, 98%, 99%, 99.5%, or 100% identical to a nucleic acid sequence provided in SEQ ID NO: 78 or 80-84, or a variant or portion thereof. In some embodiments, the nucleic acid is 600-2000, 800-1800, 1000-1600, 1200-1400 nucleotides in length. In some embodiments, the nucleic acid is 600-1600, 800-1800, 1000-2000, 2000-3000, or 3000-4000 nucleotides in length.

In some embodiments, a bifunctional SBD-BBM fusion portion can be produced in an expression vector, e.g., an expression vector comprising a nucleic acid encoding a SBD-BBM fusion protein disclosed in any of Table 1. In some embodiments, an expression vector comprises a promoter operatively linked to a nucleic acid sequence encoding a SBD-BBM fusion protein, wherein the nucleic acid sequence comprises, in any order: (a) a nucleic acid sequence encoding a Rhizavidin protein comprising amino acids of SEQ ID NO: 1 or SEQ ID NO: 2 or a protein having at least 80% sequence identity to SEQ ID NO: 1 or SEQ ID NO: 2, and (ii) a nucleic acid sequence encoding a at least one sialic acid binding domain (SBD) selected from any of: SBD1, SBD2, SBD3, SBD4, NanH, NanH2, NanH3, or VcNanH, or a SBD having an amino acid sequence that has at least 80%, or at least 85%, or at least 90%, or at least 95% sequence identity to any of: SEQ ID NO: 2-12. In some embodiments, the expression vector comprises a nucleic acid sequence encoding a Rhizavidin protein, and comprises a nucleic acid sequence having at least 80%, or at least 85%, or at least 90%, or at least 95% sequence identity to SEQ ID NO: 78 or a codon optimized variant thereof. In some embodiments, the expression vector comprises a nucleic acid sequence encoding a SBD protein of any of SBD1, SBD2, SBD3, SBD4, NanH, NanH2, NanH3, or VcNanH, and/or where the nucleic acid sequence is selected from any of: SEQ ID NO: 80-84 or a nucleic acid sequence having at least 80%, or at least 85%, or at least 90%, or at least 95% sequence identity to any of: SEQ ID NO: 80-84, or codon optimized variants thereof.

Nucleic acids encoding polypeptides or fusion proteins of Table 1, or fragments thereof, can be cloned into any of a variety of expression vectors, under the control of a variety of regulatory elements, and fusions can be created with other sequences of interest. Methods of cloning nucleic acids are routine and conventional in the art. For general references describing methods of molecular biology which are mentioned in this application, e.g., isolating, cloning, modifying, labeling, manipulating, sequencing and otherwise treating or analyzing nucleic acids and/or proteins, see, e.g., Sambrook et al, 1989; Ausubel et al, 1995; Davis et al, 1986; Hames et al, 1985; Dracopoli et al, 2018; and Coligan et al, 2018.

In all cases, due to degeneracy in the genetic code, other DNA sequences (including multiple codon-optimized sequences) could be contemplated by those of ordinary skill to encode such polypeptides and fusion proteins.

Other aspects of the technology relate to cells comprising such expression vectors encoding SBD-BBM fusion proteins as disclosed herein and methods for producing the SBD-BBM fusion proteins for use in a MAPS-X immunogenic complex, or vaccine compositions comprising a plurality of MAPS-X immunogenic complexes.

### Nucleic Acid-Based Immunogenic Compositions and Vaccines

The present disclosure also provides immunogenic compositions (e.g., vaccine compositions) of, or comprising, one or more nucleic acids encoding SBD-BBM fusion proteins described herein. In some embodiments, the immunogenic composition comprises one or more nucleic acids encoding fusion proteins with at least 80%, 85%, 90%, 95%, 96%, 97%, 98%, 99%, 99.5%, or 100% sequence identity to a fusion protein listed in Table 1. In all cases, due to degeneracy in the genetic code, other DNA sequences (including multiple codon-optimized sequences) could encode such fusion proteins. In some embodiments, these nucleic acids are expressed in the immunized individual, resulting in production of the encoded fusion proteins, and the produced fusion have an immunostimulatory or immunoprotective effect in the immunized individual.

Such a nucleic acid-containing immunostimulatory composition may comprise, for example, an origin of replication, and/or a promoter that drives expression of one or more nucleic acids encoding one or more fusion proteins disclosed in Table 1. Such a composition may also comprise a bacterial plasmid vector into which is inserted a promoter (sometimes a strong viral promoter), one or more nucleic acids encoding one or more fusion proteins of disclosed in Table 1, and a polyadenylation/transcriptional termination sequence. In some instances, the nucleic acid is DNA. In some instances, the nucleic acid is RNA.

### V. Multivalent MAPS-X Immunogenic Compositions and Vaccines

Another aspect of the disclosure provides a composition, e.g., immunogenic composition or vaccine composition that include one or more MAPS-X immunogenic complexes described herein. For example, an immunogenic composition, e.g., vaccine composition, can include one or more MAPS-X immunogenic complexes described herein. In some embodiments, such compositions can include a plurality of one type of MAPS-X immunogenic complex described herein. For example, a composition can include a population of one type of MAPS-X immunogenic complex, where all of the MAPS-X immunogenic complexes include the same antigenic polypeptide and the same antigenic polysaccharide. Additionally or alternatively, such compositions can include a plurality of more than one type of MAPS-X immunogenic complex described herein. For example, a composition can include populations of different types of MAPS-X immunogenic complexes. In some embodiments, a composition can include a population of a first type of MAPS-X immunogenic complex and a population of a second type of MAPS-X immunogenic complex, where the first type and the second type of the MAPS-X immunogenic complex have different antigenic polypeptides and/or different antigenic polysaccharides. In some embodiments, a composition can include a population of a first type of MAPS-X immunogenic complex and a population of a second type of immunogenic complex, where the first type and the second type of the MAPS-X immunogenic complex include the same antigenic polypeptide and different antigenic polysaccharides (e.g., polysaccharides of different serotypes). In some embodiments, MAPS-X immunogenic complexes described herein are formulated into a pharmaceutical composition. In some embodiments a pharmaceutical composition may be a vaccine. In some embodiments a pharmaceutical composition comprises a pharmaceutically acceptable carrier. In some embodiments a pharmaceutical composition comprises an adjuvant.

Aspects of the technology disclosed herein also provides immunogenic compositions (e.g., vaccine compositions) of, or comprising, one or more MAPS-X immunogenic complexes as described herein. In some embodiments, the immunogenic composition comprises one or more MAPS-X immunogenic complexes comprising at least one or more fusion proteins that have at least 80%, 85%, 90%, 95%, 96%, 97%, 98%, 99%, 99.5%, or 100% sequence identity to a fusion protein listed in Table 1.

Aspects of the technology disclosed herein also relate to MAPS-X immunogenic compositions (e.g., vaccine compositions) of, or comprising, one or more fusion proteins as described herein. In some embodiments, the MAPS-X immunogenic composition comprises one or more fusion proteins with at least 80%, 85%, 90%, 95%, 96%, 97%, 98%, 99%, 99.5%, or 100% sequence identity to a fusion protein comprising, or consisting of a SBD-BBM fusion protein selected from any of: SEQ ID NO: 13-20. In some embodiments, the immunogenic composition comprises a fusion protein that is or includes an amino acid sequence having at least 80%, 85%, 90%, 95%, 97%, 98%, 99%, 99.5%, or 100% identity to any one of SBD-BBM fusion protein s selected from any of SEQ ID NOS: 12-20.

In some embodiments, an immunogenic composition may also comprise portions of fusion proteins described herein, for example internal deletion mutants, truncation mutants, and fragments. In some embodiments, the portions of said fusion proteins are immunogenic. The immunogenicity of a portion of a fusion protein is readily determined using the same assays that are used to determine immunogenicity of the full-length fusion protein. In some embodiments, the portion of the fusion protein has substantially the same immunogenicity as the full-length fusion protein. In some embodiments, the immunogenicity is no less than 10%, 20%, 30%, 40%, or 50% that of the fusion proteins of listed in Table 1 or 2 herein.

### Multivalent MAPS-X Vaccine Composition

For illustrative purposes only, referring to FIGS. 1B, 1E and 1F, one aspect of the present invention relates to a composition comprising multiple species of MAPS-X immunogenic complexes. For example, in some embodiments, each species of MAPS-X immunogenic complex comprises; (i) at least a first biotinylated polysaccharide antigen (PS1) comprising at least one sialic acid molecule and at least one biotin molecule, (ii) at least a second biotinylated polysaccharide antigen (PS2), optionally comprising at least one sialic acid molecule, and (iii) at least one bifunctional SBD-BBM fusion protein, comprising at least a biotin-biotin moiety (BBM) and a sialic acid binding domain (SBD), and wherein a BBM of at least one fusion protein non-covalently associates with at least one biotin molecule on the first biotinylated polysaccharide antigen (PS1) and the SBD non-covalently associates with the PS2, e.g., but not limited to an association with at least one sialic acid on the second biotinylated polysaccharide antigen (PS2). If the MAPS-X immunogenic complex comprises more than one bifunctional SBD-BBM fusion protein, e.g., a first fusion protein (SBD-BBM fusion1) and a second bifunctional fusion protein (e.g., SBD-BBM fusion 2), then the BBM of at least a first fusion protein (SBD-BBM fusion1) non-covalently associates with at least one biotin molecule on the first biotinylated polysaccharide antigen (PS1) and the SBD non-covalently associates with at least one sialic acid on the second biotinylated polysaccharide antigen (PS2), and the SBD of a second SBD-BBM fusion protein (SBD-BBM fusion2) non-covalently associates with at least one sialic acid molecule on the first biotinylated polysaccharide antigen (PS1) and the BBM of the second fusion protein non-covalently associates with at least biotin molecule on the second biotinylated polysaccharide antigen (PS2). This results in the PS1 and PS2 forming a MAPS-X complex by the non-covalent association via the first and second bifunctional SBD-BBM fusion proteins.

Accordingly, in some embodiments, a MAPS-X immunogenic complex can comprise the following non-covalent associations; PS1-(SBD-BBM fusion protein1)-PS2, where the BBM of the first SBD-BBM fusion protein non-covalently associates with a biotin on the first polysaccharide antigen (PS1), and the SBD of the first SBD-BBM fusion protein non-covalently associates with a sialic acid on the second polysaccharide (PS2), to form an immunogenic complex. In some embodiments, the bifunctional SBD-BBM fusion protein in the MAPS-X complex is a SBD-[Ag]n-BBM fusion protein. In some embodiments, the MAPS-X complex further comprises an additional fusion protein, e.g., a SBD-[Ag]n fusion protein, and/or a BBM-[Ag]n fusion protein, as disclosed herein.

In some embodiment, a MAPS-X immunogenic complex comprises at least two SBD-BBM fusion proteins, where at least one of these bifunctional fusion proteins is a SBD-[Ag]n-BBM fusion protein—that is, at least one of the SBD-BBM fusion proteins comprise a polypeptide antigen from a pathogen as disclosed herein. Such a SBD-[Ag]n-BBM fusion protein can be selected from any fusion protein listed in Table 2 as disclosed herein. In some embodiments, a MAPS-X immunogenic complex can further comprise a SBD-BBM fusion protein that is not a bifunctional fusion protein (e.g., not SBD-BBM fusion protein or not a SBD-[Ag]n-BBM) (e.g., see FIG. 1D). Accordingly, in some embodiments, the second fusion protein in a MAPS-X immunogenic complex can be selected from any of the biotin-binding moiety (BBM) fusion protein (e.g., BBM-[Ag]n fusion protein), or (ii) a sialic acid binding molecule (SBD) fusion protein (e.g., SBD-[Ag]n fusion protein). Exemplary fusion proteins for use as a second fusion protein can be selected from any in the list disclosed in Table 2.

Exemplary configurations of MAPS-X immunogenic complexes are shown in Table 3.

TABLE 3
Exemplary MAPS-X immunogenic complexes can be as follows,
where “s” refers to sialic acid:

| | Exemplary MAPS-X | | |
|---|---|---|---|
| | immunogenic complex | Non-covalent associations | |
| 1 | [PS1-biotin]-[first | BBM of the first SBD-BBM | Such a MAPS-X |
| bifunctional | BBM-SBD]-[s-PS2] | fusion protein | complex can further |
| fusion protein | | ([BBM-SBD]) | comprise additional non- |
| | | non-covalently associates | bifunctional fusion |
| | | with a biotin on the first | proteins, e.g., a BBM- |
| | | polysaccharide antigen | [GBS-Ag]n fusion |
| | | (PS1), and the SBD of the | protein or a SBD-[GBS- |
| | | first SBD-BBM fusion | Ag]n fusion protein |
| | | protein non-covalently | |
| | | associates with a sialic | |
| | | acid on the second | |
| | | polysaccharide antigen | |
| | | (PS2) | |
| 2 | [PS1-biotin]-[first | BBM of the first SBD- | [BBM-SBD1] and |
| bifunctional | BBM-SBD]-[s-PS2] | BBM fusion protein | [BBM-SBD2] can be a |
| fusion protein | [PS1-s]-[second | ([BBM-SBD1]) non- | SBD-[Ag]n-BBM fusion |
| | SBD-BBM]-[b-PS2] | covalently associates with a | protein. They can be the |
| | | biotin on the first | same (e.g., same |
| | | biotinylated polysaccharide | species), or different |
| | | (PS1), and the SBD of the | (e.g., the GBS antigen |
| | | first SBD-BBM fusion | can be different, or in a |
| | | protein non-covalently | different arrangement |
| | | associates with a sialic acid | etc.). If they are |
| | | on the second biotinylated | different, one can be |
| | | polysaccharide (PS2) | SBD-BBM and one can |
| | | SBD of the second SBD- | be SBD-[Ag]n-BBM |
| | | BBM fusion protein | fusion protein. |
| | | ([BBM-SBD2]) non- | |
| | | covalently associates with a | |
| | | sialic acid on the first | |
| | | biotinylated polysaccharide | |
| | | (PS1), and the BBM of the | |
| | | second SBD-BBM fusion | |
| | | protein non-covalently | |
| | | associates with a biotin on | |
| | | the second biotinylated | |
| | | polysaccharide (PS2). | |

In some embodiments, a MAPS-X immunogenic complex comprises a plurality of at least 1 species of SBD-BBM fusion proteins—that is, there are plurality of the same SBD-BBM fusion in the complex (e.g. see FIG. 1C(ii)). Such a MAPS-X can comprise at least 2, 3, 4, 5, 6, 7, 8, 10, 11, 12, 13, 14, 15, 16-20, 21-30, 31-40, 41-50, 51-60, 61-70, 71-80, 81-90, 91-100 or more of the same species of bifunctional SBD-BBM fusion protein that can attach to a first and second polysaccharides. As the number of bifunctional SBD-BBM fusion increases, it can associate with more polysaccharides forming a lager MAPS-X complex. For example, referring to FIG. 1E and for simplicity and illustrative purposes only, if a MAPS-X complex comprises 5 polysaccharides, a first fusion protein (SBD-BBM fusion protein 1) can associate with PS1 and PS2, and a second fusion protein (SBD-BBM fusion protein2) can associate with PS2 and PS3, and a third fusion protein (SBD-BBM fusion protein3) can associate with, e.g., PS3 and PS4, and a fourth fusion protein (SBD-BBM fusion protein4) can associate with, e.g., PS4 and PS5, etc. thereby forming a multi-bifunctional fusion protein and polysaccharide complex. In some embodiments, the additional SBD-BBM fusion proteins can also associate with polysaccharides already existing in the complex, for example, a SBD-BBM fusion protein5 can associate with PS3 and PS5, a SBD-BBM fusion protein6 can associate with PS1 and PS3, strengthening the MAPS-X complex. It is envisioned that each MAPS-X immunogenic complex comprises a plurality of biotinylated polysaccharides, e.g., at least 5, 10, 15, 20, 25, 30, 35, 40, 45, 50, 55, 60, 65, 75, 85, 90, 95, 100 or more than 100 polysaccharide molecules which are cross linked as using a bifunctional or SBD-BBM fusion protein and/or a SBD-[Ag]-BBM fusion protein, as disclosed herein.

In such an embodiment, the polysaccharides (e.g., PS1, PS2, PS3, PS4, PS5 etc.) can be the same species, e.g., from the same serotype or different serotypes of a pathogen, e.g., bacteria. In some embodiments, the bifunctional SBD-BBM fusion protein can be the same species (e.g., comprise the same polypeptide antigen in the same configuration), or can be a different species (e.g., comprise the same polypeptide antigen in different arrangement, or alternatively, be a different polypeptide antigen).

For example, in some embodiments, all the polysaccharides (e.g., PS1, PS2, PS3, PS4, PS5 etc.) for a species of a MAPS-X immunogenic complex can be from a specific serotype of a pathogen, e.g., using Streptococcus pneumoniae as an exemplary example, all the polysaccharides in a species of MAPS-X complex can be selected from a Streptococcus pneumoniae polysaccharide having a serotype selected from one or more of 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48, e.g., see FIG. 1E. In alternative embodiments, the polysaccharides in a species of MAPS-X complex can be selected from different serotypes from the same bacteria, e.g., PS1 and PS2 can be selected from Streptococcus pneumoniae polysaccharide, e.g., at least 2, 3, 4, 5, 6, 7, 8, 9 or 10 different types of GBS polysaccharides, e.g., selected from serotypes from one or more of 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.

In some embodiments, one species of MAPS-X immunogenic complex comprises a plurality of polysaccharides (e.g., PS1, PS2, PS3, PS4, PS5 etc.) that are the same species (e.g., are from the same pathogen serotype), and a plurality of bifunctional SBD-BBM fusion proteins that are the same (e.g., comprise the same polypeptide antigen in the same configuration). In some embodiments, another species of a MAPS-X immunogenic complex comprises a plurality of polysaccharides (e.g., PS1, PS2, PS3, PS4, PS5 etc.) that are the same species, e.g., are from the same pathogen serotype, and a plurality of bifunctional SBD-BBM fusion proteins that are different species, e.g., comprise the same polypeptide antigen in different arrangement, or alternatively, comprise one or more different polypeptide antigens.

In alternative embodiments, another species of a MAPS-X immunogenic complex comprises a plurality of polysaccharides (e.g., PS1, PS2, PS3, PS4, PS5 etc.) that are different species, e.g., are from different serotypes of the same pathogen, and a plurality of bifunctional SBD-BBM fusion proteins that are the same (e.g., can comprise the same polypeptide antigen in the same configuration). In some embodiments, another species of a MAPS-X immunogenic complex comprises a plurality of polysaccharides (e.g., PS1, PS2, PS3, PS4, PS5 etc.) that are different species, e.g., are from more than one serotype of a pathogen, and a plurality of bifunctional SBD-BBM fusion proteins that are different species, e.g., comprise the same polypeptide antigen in different arrangement, or alternatively, comprise one or more different polypeptide antigens.

In some embodiments, as disclosed herein, an immunogenic composition, e.g., vaccine composition as disclosed herein comprises multiple different species of MAPS-X immunogenic complexes as described herein, e.g., see FIG. 1F. In some embodiments, each MAPS-X immunogenic complex species can comprise a different biotinylated polysaccharide selected from any polysaccharide of a pathogen of interest, as disclosed herein.

In some embodiments, each MAPS-X immunogenic complex within the composition can comprise the same species of SBD-BBM fusion protein, or different species within the complex (e.g., see FIG. 1C(ii) and 1C(iii)) as compared to other species of MAPS-X immunogenic complex in the vaccine composition. Stated differently, there can be variability of the type of SBD-BBM fusion protein (e.g., SBD-[Ag]n-BBM fusion protein) within a species of MAPS-X immunogenic complex in a vaccine, where each species of MAPS-X complex comprises a different polysaccharide. Alternatively, in some embodiments, there can be variability of the polysaccharide within a species of MAPS-X immunogenic complex in a vaccine, where each species in a plurality of MAPS-X complex comprises the same SBD-BBM fusion proteins.

In some embodiments, a SBD-BBM fusion protein as described herein, or disclosed in Table 3 is covalently bound to another molecule. This may, for example, increase the half-life, solubility, bioavailability, or immunogenicity of the fusion protein. Molecules that may be covalently bound to the fusion protein include a carbohydrate, biotin, poly(ethylene glycol) (PEG), polysialic acid, N-propanoylated polysialic acid, nucleic acids, polysaccharides, and PLGA. There are many different types of PEG, ranging from molecular weights of below 300 g/mol to over 10,000,000 g/mol. PEG chains can be linear, branched, or with comb or star geometries. In some embodiments, the fusion protein is covalently bound to a moeity that stimulates the immune system. An example of such a moeity is a lipid moeity. In some instances, lipid moieties are recognized by a Toll-like receptor (TLR) such as TLR-2 or TLR-4, and activate the innate immune system.

### VII. Uses of Fusion Proteins

In some embodiments, a SBD-BBM fusion protein described herein does not have, or has minimal, hemolytic activity. For example, in some embodiments, the hemolytic activity of a fusion protein described herein can be established by turbidimetry (OD₄₂₀) after incubation of the fusion protein at different dilutions with red blood cells (e.g., sheep erythrocytes), to determine the protein concentration at which 50% of the red blood cells are lysed. In some such embodiments, the hemolytic activity of a fusion protein described herein can be characterized by an OD₄₂₀ of less than 0.4 or lower, including, e.g., less than 0.3, less than 0.25, less than 0.2, or lower, for a given protein concentration.

In some embodiments, as described herein a SBD-BBM can further comprise an antigenic polypeptides, and fragments and variants thereof, which are immunogenic. These polypeptides and fusion proteins may be immunogenic in mammals, for example mice, rats, guinea pigs, or humans. An antigenic polypeptide or fusion protein is typically one capable of raising a significant immune response in an assay or in a subject. The immune response may be innate, humoral, cell-mediated, or mucosal (combining elements of innate, humoral and cell-mediated immunity). For instance, an antigenic polypeptide or fusion protein may increase the amount of IL-17 produced by T cells. Alternatively or additionally, an antigenic polypeptide or fusion protein may (i) induce production of antibodies, e.g., neutralizing antibodies, that bind to the polypeptide and/or the whole bacteria, (ii) induce Th17 immunity, (iii) activate the CD4+ T cell response, for example by increasing the number of CD4+ T cells and/or increasing localization of CD4+ T cells to the site of infection or reinfection, (iv) activate the CD8+ T cell response, for example by increasing the number of CD8+ T cells and/or increasing localization of CD8+ T cells to the site of infection or reinfection, (v) activate both the CD4+ and the CD8+ response, (vi) activate CD4−/CD8− immunity, (vii) induce Th1 immunity, (viii) induce anti-microbial peptides, (ix) activate innate immunity, or any combination of the foregoing. In some embodiments, an antigenic polypeptide or fusion protein elicits production of a detectable amount of antibody specific to that antigen.

In some embodiments, a SBD-BBM fusion protein as described herein is an antigen or has antigenic properties. In some embodiments, a SBD-BBM fusion protein described herein is a carrier protein or has carrier properties. In some embodiments, a SBD-BBM fusion protein described herein is both an antigen and a carrier protein, and a cross-linking moiety. In some embodiments, a SBD-BBM fusion protein described herein, e.g., a SBD-[Ag]n BBM fusion protein, has both carrier properties and antigenic properties.

In In some embodiments, a SBD-BBM fusion proteins described herein have less than 20%, 30%, 40%, 50%, 60% or 70% identity to human auto-antigens and/or gut commensal bacteria (e.g., certain Bacteroides, Clostridium, Fusobacterium, Eubacterium, Ruminococcus, Peptococcus, Peptostreptococcus, Bifidobacterium, Escherichia, and Lactobacillus species). Examples of human autoantigens include insulin, proliferating cell nuclear antigen, cytochrome P450, and myelin basic protein.

In some embodiments, a polypeptide antigen included in a SBD-[Ag]n-BBM fusion protein described herein may comprise one or more immunogenic portions and one or more non-immunogenic portions. The immunogenic portions may be identified by various methods, including protein microarrays, ELISPOT/ELISA techniques, and/or specific assays on different deletion mutants (e.g., fragments) of the polypeptide in question. Immunogenic portions may also be identified by computer algorithms. Some such algorithms, like EpiMatrix (produced by EpiVax), use a computational matrix approach. Other computational tools for identifying antigenic epitopes include PEPVAC (Promiscuous EPitope-based VACcine, hosted by Dana Farber Cancer Institute on the world wide web at immunax.dfci.harvard.edu/PEPVAC), MHCPred (which uses a partial least squares approach and is hosted by The Jenner Institute on the world wide web at www.jenner.ac.uk/MHCPred), and Immune Epitope Database algorithms on the World Wide Web at tools.immuneepitope.org. An antigenic fragment of a polypeptide described herein comprises at least one immunogenic portion, as measured experimentally or identified by algorithm (for example, the SYFPEITHI algorithm found at www.syfpeithi.de).

### VIII. Uses of Immunogenic and Vaccine Compositions

In some embodiments, a MAPS-X immune composition or vaccine as described herein, e.g., comprising a plurality of species of MAPS-X immunogenic complexes, is characterized in that one or more of the opsonization potential or immune responses to one or more polysaccharides or polypeptides (e.g., in a SBD-[Ag]n-BBM fusion proteins) of the MAPS-X complex is increased relative to a pre-determined level, as measured by ELISA and/or by a functional antibody assay. In some embodiments, one or more of the opsonization potential or immune response to the one or more fusion proteins is increased by at least 30% or more, including, e.g., at least 40%, at least 50%, at least 60%, at least 70%, at least 80%, at least 90% or more, relative to a predetermined level, as measured by ELISA and/or by a functional antibody assay. In some embodiments, one or more of the opsonization potential or immune responses to the one or more fusion proteins is increased at least 1-fold, 2-fold, 3-fold, 4-fold, or 5-fold relative to a pre-determined level, as measured by ELISA and/or by a functional antibody assay. In some embodiments, the pre-determined level is a pre-immune level (e.g., a level observed when a subject is not immunized, or is immunized in the absence of one or more fusion proteins described herein).

In some embodiments, a MAPS-X immunogenic complex or vaccine as described herein, upon administration to a subject, induces an immune response against the pathogen from which the one or more antigenic polysaccharide originates from, including induces an immune response against one or more serotypes of a pathogen that the antigenic polysaccharide originates from. The phrases as used herein “a pathogen that an antigenic polysaccharide originates from” or “a pathogen from which the antigenic polysaccharide was derived” can be used interchangeably, and refer to a pathogen that comprises the same type of polysaccharide that is used as an antigenic polysaccharide in a species of a MAPS-X complex present in the immunogenic or vaccine composition.

In some embodiments, the immunogenic composition or vaccine, upon administration to a subject, induces a protective immune response against one or more serotypes of a pathogen that polysaccharide originates from. In some embodiments, the immune response is an antibody or B cell response. In some embodiments, the immune response is a T cell response. In some embodiments, the immune response is an innate immune response. In some embodiments, the immune response is a CD4+ T cell response, including Th1, Th2, or Th17 response, or a CD8+ T cell response, or a CD4+ and a CD8+ T cell response, or a CD4−/CD8− T cell response. In some embodiments, the immune response is an antibody or B cell response and a T cell response. In some embodiments, the immune response is an antibody or B cell response, a T cell response, and an innate immune response. In some embodiments, the immune response is a protective immune response.

In some embodiments, the immune response is to at least one antigenic polysaccharide. In some embodiments, the immune response is to at least one antigenic polypeptide (also referred to as a carrier protein) present in the SBD-[Ag]n-BBM fusion protein. In some embodiments, there is an immune response is to the antigenic polysaccharide present in MAPS-X and to the antigenic polypeptide in the SBD-Ag-BBM fusion protein (also referred to as a carrier protein) in the MAPS-X immunogenic composition.

In some embodiments, an MAPS-X immunogenic complex described herein, upon administration to a subject, induces antibody production against one or more pathogens in the subject at a level greater than a composition comprising an antigenic polysaccharide alone, or to a MAPS immunogenic complex that does not comprise a bifunctional SBD-BBM fusion protein. In some embodiments, a MAPS-X immunogenic complex described herein, upon administration to a subject, induces antibody production against one or more pathogens in the subject at level greater than a composition comprising a polypeptide antigen alone, or a MAPS immunogenic complex that does not comprise a bifunctional SBD-BBM fusion protein.

In some embodiments, a MAPS-X immunogenic composition or vaccine described herein, upon administration to a subject, induces an immune response against one or more pathogens in the subject at a level greater than a composition comprising an antigenic polysaccharide alone, or a MAPS immunogenic complex that does not comprise a bifunctional SBD-BBM fusion protein. In some embodiments, a MAPS-X immunogenic complex or vaccine described herein, upon administration to a subject, induces an immune response against one or more pathogens in the subject at a level greater than a composition comprising a polypeptide antigen alone, or a MAPS immunogenic complex that does not comprise a bifunctional SBD-BBM fusion protein. In some embodiments, a MAPS-X immunogenic complex described herein, upon administration to a subject, induces a protective immune response.

In some embodiments, a MAPS-X immunogenic complex described herein that includes one or more antigenic polysaccharides is characterized in that one or more of the opsonization potential, or immune response to one or more antigenic polysaccharides is increased relative to a predetermined level, as measured by ELISA and or by a functional antibody assay. In some embodiments, one or more of the opsonization potential, immune response to the one or more antigenic polysaccharides is increased at least 1-fold, 2-fold, 3-fold, 4-fold, or 5-fold relative to a predetermined level, as measured by ELISA and or by a functional antibody assay. In some embodiments, the predetermined level is a pre-immune level. In some embodiments, the predetermined level is a pre-immune level. In some embodiments, one or more polypeptide antigens are carrier proteins for one or more antigenic polysaccharides.

In some embodiments, a MAPS-X immunogenic complex described herein, upon administration to a subject, induces an immune response against one or more pathogens in the subject at a level greater than a composition comprising an antigenic polysaccharide alone, or a MAPS immunogenic complex that does not comprise a bifunctional SBD-BBM fusion protein. In some embodiments, a MAPS-X immunogenic complex described herein, upon administration to a subject, induces an immune response against one or more pathogens in the subject at a level greater than a composition comprising a polypeptide antigen alone, or a MAPS immunogenic complex that does not comprise a bifunctional SBD-BBM fusion protein. In some embodiments, a MAPS-X immunogenic complex described herein, upon administration to a subject, induces a protective immune response.

In some embodiments, a MAPS-X immunogenic complex described herein, upon administration to a subject, induces an immune response against a pathogen, where a MAPS-X complex comprises an antigenic polysaccharide of the pathogen. In some embodiments, a MAPS-X immunogenic complex described herein, upon administration to a subject, induces an immune response against one or more serotypes of a pathogen, where a MAPS-X complex comprises an antigenic polysaccharide of the pathogen. In some embodiments, such an immune response may be directed against one or more (e.g., 1, 2, 3, 4, 5, 6, 7, 8, 9, 10 or more) serotypes of a pathogen, where a MAPS-X complex comprises an antigenic polysaccharide of the pathogen, wherein an immunogenic complex described herein includes polysaccharide(s) present in at least one or more (e.g., 1, 2, 3, 4, 5, 6, 7, 8, 9, 10 or more) of such serotype(s). In some embodiments, such an immune response may be directed against one or more (e.g., 1, 2, 3, 4, 5, 6, 7, 8, 9, 10 or more) serotypes of a pathogen, where a MAPS-X complex comprises an antigenic polysaccharide of the pathogen, wherein an immunogenic complex described herein does not include polysaccharide(s) present in at least one or more (e.g., 1, 2, 3, 4, 5, 6, 7, 8, 9, 10 or more) of such serotype(s). In some embodiments, such an immune response may be directed against two or more (e.g., 2, 3, 4, 5, 6, 7, 8, 9, 10 or more) serotypes of a pathogen, where a MAPS-X complex comprises an antigenic polysaccharide of the pathogen, wherein an immunogenic complex described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s). In some embodiments, an immunogenic complex described herein, upon administration to a subject, induces a protective immune response against one or more serotypes of a pathogen, where a MAPS-X complex comprises an antigenic polysaccharide of the pathogen. In some embodiments, such a protective response may be directed against one or more (e.g., 1, 2, 3, 4, 5, 6, 7, 8, 9, 10 or more) serotypes of a pathogen, where a MAPS-X complex comprises an antigenic polysaccharide of the pathogen, wherein an immunogenic complex described herein includes polysaccharide(s) present in at least one or more (e.g., 1, 2, 3, 4, 5, 6, 7, 8, 9, 10 or more) of such serotype(s). In some embodiments, such a protective response may be directed against one or more (e.g., 1, 2, 3, 4, 5, 6, 7, 8, 9, 10 or more) serotypes of a pathogen, where a MAPS-X complex comprises an antigenic polysaccharide of the pathogen, wherein an immunogenic complex described herein does not include polysaccharide(s) present in at least one or more (e.g., 1, 2, 3, 4, 5, 6, 7, 8, 9, 10 or more) of such serotype(s). In some embodiments, such a protective response may be directed against two or more (e.g., 2, 3, 4, 5, 6, 7, 8, 9, 10 or more) serotypes of a pathogen, where a MAPS-X complex comprises an antigenic polysaccharide of the pathogen, wherein an immunogenic complex described herein (i) includes polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s).

The immunogenic compositions and vaccines comprising MAPS-X immunogenic complexes as described herein may be used for prophylactic and/or therapeutic treatment for a pathogen, where a MAPS-X complex comprises an antigenic polysaccharide of the pathogen. Accordingly, this application provides a method for immunizing a subject suffering from or susceptible to a pathogen infection, comprising administering an immunologically effective amount of any of the MAPS-X immunogenic compositions or vaccine formulations described herein. The subject receiving the vaccination may be a male or a female, and may be an infant, child, adolescent, or adult. In some embodiments, the subject being treated is a human. In other embodiments, the subject is a non-human animal. In some embodiments, an immunogenic complex described herein, upon administration to a subject, induces a protective immune response against one or more serotypes of a pathogen from which the antigenic polysaccharide was derived.

In prophylactic embodiments, a MAPS-X vaccine composition (e.g., ones as described and/or utilized herein) is administered to a subject to induce an immune response that can help protect against the establishment of an infection from a pathogen from which the antigenic polysaccharide was derived, for example by protecting against colonization, the first and necessary step in disease. In some embodiments, such an immune response may be directed against one or more (e.g., 1, 2, 3, 4, 5, 6, 7, 8, 9, 10 or more) serotypes of a pathogen from which the antigenic polysaccharide was derived, wherein a vaccine composition described herein includes polysaccharide(s) present in at least one or more (e.g., 1, 2, 3, 4, 5, 6, 7, 8, 9, 10 or more) of such serotype(s). In some embodiments, such an immune response may be directed against one or more (e.g., 1, 2, 3, 4, 5, 6, 7, 8, 9, 10 or more) serotypes of a pathogen from which the antigenic polysaccharide was derived, wherein a vaccine composition described herein does not include polysaccharide(s) present in at least one or more (e.g., 1, 2, 3, 4, 5, 6, 7, 8, 9, 10 or more) of such serotype(s) (non-vaccine types, NVTs). In some embodiments, such an immune response may be directed against two or more (e.g., 2, 3, 4, 5, 6, 7, 8, 9, 10 or more) serotypes of a pathogen from which the antigenic polysaccharide was derived, wherein a vaccine composition described herein (i) comprises a plurality of species of MAPS-X immunogenic complexes comprising polysaccharide(s) present in at least one or more (e.g., 1, 2, 3, 4, 5, 6, 7, 8, 9, 10 or more) of such serotype(s); and (ii) does not include polysaccharide(s) present in at least one or more (e.g., 1, 2, 3, 4, 5, 6, 7, 8, 9, 10 or more) of such serotype(s). Thus, in some aspects, the method inhibits infection by a pathogen from which the antigenic polysaccharide was derived in a noncolonized or uninfected subject. In another aspect, the method may reduce the duration of colonization in a subject who is already colonized.

In therapeutic embodiments, the MAPS-X vaccine may be administered to a subject suffering from an infection or disease caused by a pathogen from which the antigenic polysaccharide was derived, in an amount sufficient to treat the subject. Treating the subject, in this case, can relate to reducing a symptom and/or bacterial load and/or sequelae in an infected subject. In some embodiments, treating the subject refers to reducing the duration of symptoms or sequelae, or reducing the intensity of symptoms or sequelae. In some embodiments, the vaccine reduces transmissibility of the pathogen and/or infection from the vaccinated subject. In certain embodiments, the reductions described above are at least 25%, 30%, 40%, 50%, 60%, 70%, 80%, or 90%.

In therapeutic embodiments, a MAPS-X vaccine is administered to a subject postinfection. The vaccine may be administered shortly after infection, e.g., before symptoms or sequelae manifest, or may be administered during or after manifestation of symptoms or sequelae.

In some embodiments, an immunogenic composition and/or vaccine compositions as disclosed herein comprising a plurality of MAPS-X immunogenic complexes confer protective immunity, allowing a vaccinated subject to exhibit delayed onset of symptoms or sequelae, or reduced severity of symptoms or sequelae, as the result of his or her exposure to the vaccine. In certain embodiments, the reduction in severity of symptoms or sequelae is at least 25%, 40%, 50%, 60%, 70%, 80%, or 90%. In particular embodiments, vaccinated subjects may display no symptoms or sequelae upon contact with a pathogen from which the antigenic polysaccharide was derived, do not become colonized by such a pathogen, or both. Protective immunity is typically achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves macrophages and T lymphocytes, as well as other mechanisms involving T cells without a requirement for antibodies. In particular, cellular immunity may be mediated by Th1 or Th17 cells.

In some embodiments, a MAPS-X immunogenic complex or vaccine described herein is a multivalent MAPS-X vaccine, where upon administration to a subject, it induces an immune response against multiple serotypes of a pathogen from which the antigenic polysaccharide was derived. In some embodiments, a multivalent MAPS-X immunogenic complex or vaccine described herein, upon administration to a subject, induces an immune response, including but not limited to, a protective immune response, against one or more (e.g., 1, 2, 3, 4, 5, 6, 7, 8, 9, 10, 11, 12, 13, 14, 15, 16, 17, 18, 19, 20, 21, 22, 23, 24, or more) serotypes of a pathogen from which the antigenic polysaccharide was derived.

In some embodiments, a MAPS-X immunogenic complex described herein, upon administration to a subject, can induce a protective immune response against all pathogens, including all serotypes of a pathogen from which the antigenic polysaccharide in the MAPS-X complex was derived. That is, for example, if a MAPS-X vaccine or composition comprises a plurality of different MAPS-X immunogenic complexes, e.g., (i) a first MAPS-X complex (e.g., MAPS-Xa) that comprises an antigenic polysaccharide to a first pathogen (pathogen A), (ii) a second MAPS-X complex (e.g., MAPS-Xb) comprising e.g., an antigenic polysaccharide to a second pathogen (e.g., pathogen B), and (iii) a third MAPS-X complex (e.g., MAPS-Xc) comprising an antigenic polysaccharide to a third pathogen (e.g., pathogen C), a multi-pathogen MAPS-X vaccine composition as disclosed herein induces a protective immune response against all pathogens A, B and C, and in some embodiments, can induce a protective immune response to at least 1 or more serotypes (e.g., 1, 2, 3, 4, 5, 6, 7, 8, 9, 10, 11, 12, 13, 14, 15, 16, 17, 18, 19, 20, 21, 22, 23, 24, or more) serotypes of pathogen A, pathogen B and pathogen C. Accordingly, it is envisioned herein that a MAPS-X vaccine can comprise a plurality of MAPS-X immunogenic complexes, where each species of MAPS-X immune complex can comprise at least one antigenic polysaccharides selected from 1, 2, 3, 4, 5, 6, 7, 8, 9, or 10 or more different pathogens. As such, a MAPS-X vaccine or immunogenic composition as disclosed herein can induce an immune response to more than one pathogen at a time.

In addition, as disclosed herein, it is envisioned herein that a MAPS-X vaccine can comprise a plurality of MAPS-X immunogenic complexes, where each species of MAPS-X immune complex can comprise (i) antigenic polysaccharides selected from a 1, 2, 3, 4, 5, 6, 7, 8, 9, or 10 or more different serotypes of the same pathogen, and/or (ii) antigenic polysaccharides selected from a 1, 2, 3, 4, 5, 6, 7, 8, 9, or 10 or more different pathogens, thereby the MAPS-X vaccine or immunogenic composition can induce an immune response to more than one pathogen, as well as multiple serotypes of each pathogen.

By way of an example only, in some embodiments, a multivalent MAPS-X immunogenic complex or vaccine described herein, is selected from any of 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V or more than 24V MAPS-X vaccine, where V refers to valency (e.g., 7V refers to 7valent) and 1V (1 valent) refers to a single species of MAPS-X immunogenic complex, were upon administration to a subject, it induces an immune response, including but not limited to, a protective immune response, against one or more (e.g., 1, 2, 3, 4, 5, 6, 7, 8, 9, 10, 11, 12, 13, 14, 15, 16, 17, 18, 19, 20, 21, 22, 23, 24, or more) species of MAPS-X immunogenic complex. As each species of MAPS-X immunogenic complex in an immunogenic composition or vaccine as disclosed herein can comprise (i) an antigenic polysaccharide that is derived from a different pathogen to other species of MAPS-X immune complexes in the vaccine, and/or (ii) can comprise an antigenic polysaccharide that is derived from a different subtype of the same pathogen of other species of MAPS-X complexes in the composition, the immunogenic composition or vaccine comprising MAPS-X complexes is flexible to induce immune responses, e.g., including protective immune responses to multiple pathogens and/or multiple serotypes of a pathogen from which the antigenic polysaccharide was derived.

In some embodiments, the immune response is an antibody or B cell response. In some embodiments, the immune response is a T cell response. In some embodiments, the immune response is an innate immune response. In some embodiments, the immune response is a CD4+ T cell response, including Th1, Th2, or Th17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response.

In some embodiments, an immunogenic composition or vaccine described herein comprising a plurality of MAPS-X immunogenic complexes, upon administration to a subject, induces an antibody or B cell response against one or more pathogens in the subject at a level greater than a composition comprising an antigenic polysaccharide alone. In some embodiments, an immunogenic composition or vaccine described herein, upon administration to a subject, induces an antibody or B cell response against one or more pathogens in the subject at level greater than a composition comprising a polypeptide antigen alone. In some embodiments, the immune response is a protective immune response.

In some embodiments, a MAPS-X immunogenic complex or vaccine described herein, upon administration to a subject, induces a T cell response against one or more pathogens in the subject at a level greater than a composition comprising an antigenic polysaccharide alone. In some embodiments, an immunogenic composition or vaccine described herein, upon administration to a subject, induces a T cell response against one or more pathogens in the subject at level greater than a composition comprising a polypeptide antigen alone. In some embodiments, the immune response is a protective immune response.

In some embodiments, upon administration to a subject, an immunogenic composition or vaccine described herein treats or prevents infection by a pathogen from which the antigenic polysaccharide in the MAPS-X complex is derived. In some embodiments, upon administration to a subject, an immunogenic composition or vaccine described herein inhibits or reduces the rate of occurrence of infection by a pathogen from which the antigenic polysaccharide in the MAPS-X complex is derived. In some embodiments, upon administration to a subject, an immunogenic composition or vaccine described herein reduces the severity of infection by a pathogen from which the antigenic polysaccharide in the MAPS-X complex is derived. In some embodiments, upon administration to a subject, an immunogenic composition or vaccine described herein inhibits transmission of a pathogen from which the antigenic polysaccharide in the MAPS-X complex is derived from the subject to another subject.

In some embodiments, an immunogenic composition or vaccine that includes one or more MAPS-X immunogenic complexes and/or fusion proteins described herein may be used for prophylactic and/or therapeutic treatment of infection by a pathogen from which the antigenic polysaccharide in the MAPS-X complex is derived. Accordingly, the present disclosure provides a method for immunizing a subject suffering from or susceptible to a pathogen from which the antigenic polysaccharide in the MAPS-X complex is derived infection, comprising administering an immunologically effective amount of any immunogenic composition or vaccine that includes one or more MAPS-X immunogenic complexes as disclosed herein and comprising one or more fusion proteins as described herein. The subject receiving the immunization may be a male or a female, and may be an infant, child, adolescent, or adult. In some embodiments, the subject being treated is a human. In other embodiments, the subject is a non-human animal.

In some embodiments, upon administration to a subject, an immunogenic composition or vaccine comprising one or more MAPS-X immunogenic complexes as disclosed herein and comprising one or more fusion proteins as described herein treats or prevents infection by a pathogen from which the antigenic polysaccharide in the MAPS-X complex is derived. In some embodiments, upon administration to a subject, the immunogenic composition or vaccine treats or prevents Invasive Disease (IPD) due to infection by a pathogen from which the antigenic polysaccharide in the MAPS-X complex is derived. In some embodiments, upon administration to a subject, the immunogenic composition or vaccine treats or prevents bacteremia due to infection by a pathogen from which the antigenic polysaccharide in the MAPS-X complex is derived. In some embodiments, upon administration to a subject, the immunogenic composition or vaccine treats or prevents any of: sepsis, pneumonia, and meningitis due to infection by a pathogen from which the antigenic polysaccharide in the MAPS-X complex is derived. In some embodiments, upon administration to a subject, the immunogenic composition or vaccine treats or prevents organ damage due to infection by a pathogen from which the antigenic polysaccharide in the MAPS-X complex is derived. In some embodiments, upon administration to a subject, the immunogenic composition or vaccine treats or prevents meningitis due to infection by a pathogen from which the antigenic polysaccharide in the MAPS-X complex is derived. In some embodiments, upon administration to a subject, the immunogenic composition or vaccine treats or prevents pneumonia due to infection by a pathogen from which the antigenic polysaccharide in the MAPS-X complex is derived. In some embodiments, upon administration to a subject, the immunogenic composition or vaccine treats or prevents otitis media due to infection by a pathogen from which the antigenic polysaccharide in the MAPS-X complex is derived. In some embodiments, upon administration to a subject, the immunogenic composition or vaccine treats or prevents sinusitis due to infection by a pathogen from which the antigenic polysaccharide in the MAPS-X complex is derived.

In some embodiments, upon administration to a female pregnant subject, the immunogenic composition or vaccine treats or prevents any of: sepsis, pneumonia, and meningitis in the baby in utero or post partum, due to infection by a pathogen, e.g. a pathogen from which the antigenic polysaccharide in the MAPS-X complex is derived.

In some embodiments, upon administration to a subject, an immunogenic composition or vaccine comprising a MAPS-X immunogenic complex as described herein inhibits or reduces the rate of occurrence of infection of a human baby by a pathogen from which the antigenic polysaccharide in the MAPS-X complex is derived. In some embodiments, upon administration to a subject, including pregnant females, babies and the elderly, the immunogenic composition or vaccine inhibits or reduces the rate of occurrence of Invasive Disease (IPD) due to infection by a pathogen, e.g., a pathogen from which the antigenic polysaccharide in the MAPS-X complex is derived. In some embodiments, upon administration to a subject, the immunogenic composition or vaccine inhibits or reduces the rate of occurrence of bacteremia due to infection by a pathogen from which the antigenic polysaccharide in the MAPS-X complex is derived. In some embodiments, upon administration to a subject, the immunogenic composition or vaccine inhibits or reduces the rate of occurrence of sepsis due to infection by a pathogen from which the antigenic polysaccharide in the MAPS-X complex is derived. In some embodiments, upon administration to a subject, the immunogenic composition or vaccine inhibits or reduces the rate of occurrence of organ damage due to infection by a pathogen from which the antigenic polysaccharide in the MAPS-X complex is derived. In some embodiments, upon administration to a subject, the immunogenic composition or vaccine inhibits or reduces the rate of occurrence of meningitis due to infection by a pathogen from which the antigenic polysaccharide in the MAPS-X complex is derived. In some embodiments, upon administration to a subject, the immunogenic composition or vaccine inhibits or reduces the rate of occurrence of pneumonia due to infection by a pathogen from which the antigenic polysaccharide in the MAPS-X complex is derived. In some embodiments, upon administration to a subject, the immunogenic composition or vaccine inhibits or reduces the rate of occurrence of otitis media due to infection by a pathogen from which the antigenic polysaccharide in the MAPS-X complex is derived. In some embodiments, upon administration to a subject, the immunogenic composition or vaccine inhibits or reduces the rate of occurrence of sinusitis due to infection by a pathogen from which the antigenic polysaccharide in the MAPS-X complex is derived.

In some embodiments, upon administration to a subject, an immunogenic composition or vaccine comprising a plurality of MAPS-X immunogenic complexes as described herein reduces the severity of infection by a pathogen from which the antigenic polysaccharide in the MAPS-X complex is derived. In some embodiments, upon administration to a subject, the immunogenic composition or vaccine reduces the severity of Invasive a pathogen from which the antigenic polysaccharide in the MAPS-X complex is derived Disease (IPD) due to infection by a pathogen from which the antigenic polysaccharide in the MAPS-X complex is derived. In some embodiments, upon administration to a subject, the immunogenic composition or vaccine reduces the severity of bacteremia due to infection by a pathogen from which the antigenic polysaccharide in the MAPS-X complex is derived. In some embodiments, upon administration to a subject, the immunogenic composition or vaccine reduces the severity of sepsis due to infection by a pathogen from which the antigenic polysaccharide in the MAPS-X complex is derived. In some embodiments, upon administration to a subject, the immunogenic composition or vaccine reduces the severity of organ damage due to infection by a pathogen from which the antigenic polysaccharide in the MAPS-X complex is derived. In some embodiments, upon administration to a subject, the immunogenic composition or vaccine reduces the severity of meningitis due to infection by a pathogen from which the antigenic polysaccharide in the MAPS-X complex is derived. In some embodiments, upon administration to a subject, the immunogenic composition or vaccine reduces the severity of pneumonia due to infection by a pathogen from which the antigenic polysaccharide in the MAPS-X complex is derived. In some embodiments, upon administration to a subject, the immunogenic composition or vaccine reduces the severity of otitis media due to infection by a pathogen from which the antigenic polysaccharide in the MAPS-X complex is derived. In some embodiments, upon administration to a subject, the immunogenic composition or vaccine reduces the severity of sinusitis due to infection by a pathogen from which the antigenic polysaccharide in the MAPS-X complex is derived.

In some embodiments, upon administration to a subject, an immunogenic composition or vaccine comprising a plurality of MAPS-X immunogenic complexes as described herein inhibits transmission of a pathogen from which the antigenic polysaccharide in the MAPS-X complex is derived from the subject to another subject. In some embodiments, upon administration to a subject, the immunogenic composition or vaccine inhibits colonization by a pathogen from which the antigenic polysaccharide in a MAPS-X complex of the vaccine is derived in the subject. In some embodiments, upon administration to a subject, the immunogenic composition or vaccine inhibits colonization by a pathogen from which the antigenic polysaccharide in a MAPS-X complex of the vaccine is derived in the nasopharynx of the subject.

In some embodiments, an immunogenic composition or vaccine comprising a MAPS-X immunogenic complex as described herein, upon administration to a subject, induces an immune response against a pathogen, e.g., where a MAPS-X complex comprises an antigenic polysaccharide of the pathogen in the subject at a level greater than a control composition. In some embodiments, the immunogenic composition or vaccine, upon administration to a subject, induces an immune response against one or more serotypes of a pathogen, where the pathogen comprises the same type of polysaccharide present in the MAPS-X complex, at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition or vaccine comprising a plurality of MAPS-X immunogenic complexes as described herein, upon administration to a subject, induces an immune response that can help protect against the establishment of infection by a pathogen, e.g., a pathogen from which the antigenic polysaccharide was derived, at a level greater than a control composition. In some embodiments, the immunogenic composition or vaccine protects against colonization at a level greater than a control composition. In some embodiments, the immunogenic composition or vaccine inhibits infection by a pathogen, e.g., a pathogen from which the antigenic polysaccharide was derived, in a non-colonized or uninfected subject at a level greater than a control composition. In some embodiments, the immunogenic composition or vaccine reduces the duration of colonization by a pathogen, e.g., a pathogen from which the antigenic polysaccharide was derived, in a subject who is already colonized at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, a control composition is, e.g., a polysaccharide vaccine to a pathogen, including polysaccharide-protein conjugates that have covalent linkages (e.g., a non-MAPS GBS vaccine), e.g., as disclosed in WO2016178123A1 or a MAPS vaccine comprising MAPS complexes that do not comprise a bifunctional SBD-BBM fusion protein.

### IX. Antibody Compositions

Some embodiments provide for an antibody composition comprising antibodies raised in a mammal immunized with an immunogenic composition or vaccine comprising a MAPS-X immunogenic complex as described herein. In some embodiments, an antibody comprises at least one antibody selected from the group consisting of monoclonal Abs (mAbs) and anti-idiotype antibodies. In some embodiments, an antibody composition comprises an isolated gamma globulin fraction. In some embodiments, an antibody composition comprises polyclonal antibodies. In some embodiments, the antibody composition is administered to a subject. In some embodiments, the antibody composition administered to a subject confers passive immunization.

### X. Vaccine Formulations

In some embodiments, an immunogenic composition, e.g., a vaccine composition is a polyvalent or multivalent MAPS-X vaccine. In some embodiments, the valency of a vaccine composition refers to the number of species of MAPS-X immunogenic complexes present in the vaccine composition. The valency of a vaccine described herein is not limiting with respect to the total antigens present in said pharmaceutical composition, immunogenic complex, or vaccine, or to the number of pathogen strains for which administration of said pharmaceutical composition, immunogenic complex, immunogenic composition, or vaccine composition may induce an immune-protective response. In a non-limiting example, a 6-valent (6V) vaccine composition may comprise more than 6 antigenic components (e.g., peptide and/or polysaccharide components) and may induce an immunoprotective response against more than 6 pathogens, or pathogenic serotypes or strains.

In some embodiments, a vaccine composition comprises between 1-50 species of MAPS-X immunogenic complexes. In some embodiments, a vaccine composition comprises between 1-40 species of MAPS-X immunogenic complexes. In some embodiments, a vaccine composition comprises between 1-35 species of MAPS-X immunogenic complexes. In some embodiments, a vaccine composition comprises between 1-30 species of MAPS-X immunogenic complexes. In some embodiments, a vaccine composition comprises between 1-30 species of MAPS-X immunogenic complexes. In some embodiments, a vaccine composition comprises between 1-24 species of MAPS-X immunogenic complexes. In some embodiments, a vaccine composition comprises between 1-15 species of MAPS-X immunogenic complexes. In some embodiments, a vaccine composition comprises between 1-9 species of MAPS-X immunogenic complexes. In some embodiments, a vaccine composition comprises between 1-5 species of MAPS-X immunogenic complexes. In some embodiments, a vaccine is a polyvalent vaccine.

In some embodiments, a vaccine composition comprises two or more species of MAPS-X immunogenic complexes (e.g., in immunogenic compositions) in amounts such that the weight of polysaccharides in the vaccine composition from each MAPS-X immunogenic complex is about the same, e.g., present in a w/w ratio of about 1:1. In some embodiments, the weight of polysaccharides in the vaccine contributed by each MAPS-X immunogenic complex is about 0.20 ug. In some embodiments, the weight of polysaccharides in the vaccine contributed by each MAPS-X immunogenic complex is about 0.25 ug. In some embodiments, the weight of polysaccharides in the vaccine contributed by each MAPS-X immunogenic complex is about 0.5 ug. In some embodiments, the weight of polysaccharides in the vaccine contributed by each MAPS-X immunogenic complex is about 1 ug. In some embodiments, the weight of polysaccharides in the vaccine contributed by each MAPS-X immunogenic complex is about 1.5 ug. In some embodiments, the weight of polysaccharides in the vaccine contributed by each MAPS-X immunogenic complex is about 2 ug. In some embodiments, the weight of polysaccharides in the vaccine contributed by each MAPS-X immunogenic complex is about 2.5 ug. In some embodiments, the weight of polysaccharides in the vaccine contributed by each MAPS-X immunogenic complex is about 3 ug. In some embodiments, the weight of polysaccharides in the vaccine contributed by each MAPS-X immunogenic complex is about 3.5 ug. In some embodiments, the weight of polysaccharides in the vaccine contributed by each MAPS-X immunogenic complex is about 4 ug. In some embodiments, the weight of polysaccharides in the vaccine contributed by each MAPS-X immunogenic complex is about 4.5 ug. In some embodiments, the weight of polysaccharides in the vaccine contributed by each MAPS-X immunogenic complex is about 5 ug. In some embodiments, the weight of polysaccharides in the vaccine contributed by each MAPS-X immunogenic complex is about 5.5 ug. In some embodiments, the weight of polysaccharides in the vaccine contributed by each MAPS-X immunogenic complex is about 6 ug. In some embodiments, the weight of polysaccharides in the vaccine contributed by each MAPS-X immunogenic complex is about 7 ug. In some embodiments, the weight of polysaccharides in the vaccine contributed by each MAPS-X immunogenic complex is about 8 ug. In some embodiments, the weight of polysaccharides in the vaccine contributed by each MAPS-X immunogenic complex is about 9 ug. In some embodiments, the weight of polysaccharides in the vaccine contributed by each MAPS-X immunogenic complex is about 10 ug. In some embodiments, the weight of polysaccharides in the vaccine contributed by each MAPS-X immunogenic complex is about 11 ug. In some embodiments, the weight of polysaccharides in the vaccine contributed by each MAPS-X immunogenic complex is about 12 ug. In some embodiments, the weight of polysaccharides in the vaccine contributed by each MAPS-X immunogenic complex is more than 12 ug, e.g., 13 ug, 14 ug, 15 ug, 16 ug, 17 ug, 18 ug, 19 ug, 20 ug, 21 ug, 22 ug, 23 ug, 24 ug, 25 ug, or more.

In some embodiments, a vaccine composition comprises two or more species of MAPS-X immunogenic complexes (e.g., in immunogenic compositions) in amounts such that the weight of polysaccharides in the vaccine composition contributed by each MAPS-X immunogenic complex is different, e.g., present in a w/w ratio that is not about 1:1. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:2. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:3. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:4. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:5. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:6. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:7. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:8. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:9. In some embodiments, a vaccine composition comprises two or more species of MAPS-X immunogenic complexes (e.g., in immunogenic compositions) in amounts such that the weight of polysaccharide in the vaccine composition contributed by a first MAPS-X immunogenic complex and a second MAPS-X immunogenic complex is 1:10. In some embodiments, the vaccine composition comprises a mixture of MAPS-X immunogenic complexes, such that the weight of polysaccharide in a vaccine contributed by an MAPS-X immunogenic complex ranges from about 0.20 ug to about 6 ug. In some embodiments, the vaccine composition comprises a mixture of MAPS-X immunogenic complexes, such that the weight of polysaccharide in a vaccine contributed by an MAPS-X immunogenic complex ranges from about 0.20 ug to about 12 ug. In some embodiments, the vaccine composition comprises a mixture of MAPS-X immunogenic complexes, such that the weight of polysaccharides in the vaccine contributed by each MAPS-X immunogenic complex ranges from about 0.20 ug to about 20 ug. In some embodiments, the vaccine composition comprises a mixture of MAPS-X immunogenic complexes, such that the weight of polysaccharides in the vaccine contributed by each MAPS-X immunogenic complex ranges from about 0.20 ug to about 40 ug.

In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is about the same, e.g., present in a w/w protein:PS ratio of about 1:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 2:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 3:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 4:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 5:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 6:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 7:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 8:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 9:1. In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex (e.g., in an immunogenic composition) is present in a w/w protein:PS ratio of about 10:1.

In some embodiments, the combined weight of polysaccharides and polypeptides in the vaccine contributed by each MAPS-X immunogenic complex is about 0.20 ug. In some embodiments, the combined weight of polysaccharides and polypeptides in the vaccine contributed by each MAPS-X immunogenic complex is about 0.40 ug. In some embodiments, the combined weight of polysaccharides and polypeptides in the vaccine contributed by each MAPS-X immunogenic complex is about 1 ug. In some embodiments, the combined weight of polysaccharides and polypeptides in the vaccine contributed by each MAPS-X immunogenic complex is about 2 ug. In some embodiments, the combined weight of polysaccharides and polypeptides in the vaccine contributed by each immunogenic composition is about 3 ug. In some embodiments, the combined weight of polysaccharides and polypeptides in the vaccine contributed by each immunogenic composition is about 4 ug. In some embodiments, the combined weight of polysaccharides and polypeptides in the vaccine contributed by each immunogenic composition is about 5 ug. In some embodiments, the combined weight of polysaccharides and polypeptides in the vaccine contributed by each immunogenic composition is about 6 ug. In some embodiments, the combined weight of polysaccharides and polypeptides in the vaccine contributed by each immunogenic composition is about 7 ug. In some embodiments, the combined weight of polysaccharides and polypeptides in the vaccine contributed by each immunogenic composition is about 8 ug. In some embodiments, the combined weight of polysaccharides and polypeptides in the vaccine contributed by each immunogenic composition is about 9 ug. In some embodiments, the combined weight of polysaccharides and polypeptides in the vaccine contributed by each immunogenic composition is about 10 ug. In some embodiments, the combined weight of polysaccharides and polypeptides in the vaccine contributed by each immunogenic composition is about 11 ug. In some embodiments, the combined weight of polysaccharides and polypeptides in the vaccine contributed by each immunogenic composition is about 12 ug. In some embodiments, the combined weight of polysaccharides and polypeptides in the vaccine contributed by each immunogenic composition is about 14 ug. In some embodiments, the combined weight of polysaccharides and polypeptides in the vaccine contributed by each immunogenic composition is about 16 ug. In some embodiments, the combined weight of polysaccharides and polypeptides in the vaccine contributed by each immunogenic composition is about 18 ug. In some embodiments, the combined weight of polysaccharides and polypeptides in the vaccine contributed by each immunogenic composition is about 20 ug. In some embodiments, the combined weight of polysaccharides and polypeptides in the vaccine contributed by each immunogenic composition is about 21 ug. In some embodiments, the combined weight of polysaccharides and polypeptides in the vaccine contributed by each immunogenic composition is about 22 ug. In some embodiments, the combined weight of polysaccharides and polypeptides in the vaccine contributed by each immunogenic composition is about 23 ug. In some embodiments, the combined weight of polysaccharides and polypeptides in the vaccine contributed by each immunogenic composition is about 24 ug. In some embodiments, the combined weight of polysaccharides and polypeptides in the vaccine contributed by each immunogenic composition is about 25 ug. In some embodiments, the combined weight of polysaccharides and polypeptides in the vaccine contributed by each immunogenic composition is about 30 ug. In some embodiments, the combined weight of polysaccharides and polypeptides in the vaccine contributed by each immunogenic composition is about 40 ug. In some embodiments, the combined weight of polysaccharides and polypeptides in the vaccine contributed by each immunogenic composition is about 50 ug. In some embodiments, the combined weight of polysaccharides and polypeptides in the vaccine contributed by each immunogenic composition is about 60 ug. In some embodiments, the combined weight of polysaccharides and polypeptides in the vaccine contributed by each immunogenic composition is about 70 ug. In some embodiments, the combined weight of polysaccharides and polypeptides in the vaccine contributed by each immunogenic composition is about 80 ug. In some embodiments, the combined weight of polysaccharides and polypeptides in the vaccine contributed by each immunogenic composition is about 90 ug. In some embodiments, the combined weight of polysaccharides and polypeptides in the vaccine contributed by each immunogenic composition is about 100 ug. In some embodiments, the combined weight of polysaccharides and polypeptides in the vaccine contributed by each immunogenic composition is about 110 ug.

In some embodiments, a vaccine composition comprises two or more species of MAPS-X immunogenic complexes (e.g., in immunogenic compositions) in amounts such that the combined weight of polysaccharides and polypeptides in the vaccine composition contributed by each MAPS-X immunogenic complex is different, e.g., present in a w/w protein:PS ratio that is not about 1:1, e.g., a protein:PS ratio that is 2:1, 3:1, 4:1. 5:1. 6:1, 7:1, 8:1, 9:1, or 10:1. In some embodiments, the vaccine composition comprises a mixture of MAPS-X immunogenic complexes, such that the combined weight of polysaccharides and polypeptides in the vaccine contributed by each MAPS-X immunogenic complex ranges from about 0.4 ug to about 110 ug.

Optimal amounts of components for a particular MAPS-X vaccine comprising a plurality of MAPS-X immunogenic complexes, and/or fusion proteins as described herein can be ascertained by standard studies involving observation of appropriate immune responses in subjects. Following an initial immunization, subjects can receive one or several booster immunizations adequately spaced in time.

The immunogenic composition or vaccine comprising a plurality of MAPS-X immunogenic complexes, and/or fusion proteins as described herein, and/or preparations thereof, may be formulated in a unit dosage form for ease of administration and uniformity of dosage. The specific therapeutically effective dose level for any particular subject or organism may depend upon a variety of factors including the severity or degree of risk of infection; the activity of the specific vaccine or vaccine composition employed; other characteristics of the specific vaccine or vaccine composition employed; the age, body weight, general health, sex of the subject, diet of the subject, pharmacokinetic condition of the subject, the time of administration (e.g., with regard to other activities of the subject such as eating, sleeping, receiving other medicines including other vaccine doses, etc.), route of administration, rate of excretion of the specific vaccine or vaccine composition employed; vaccines used in combination or coincidental with the vaccine composition employed; and like factors well known in the medical arts.

An immunogenic composition or vaccine comprising a plurality of MAPS-X immunogenic complexes and/or fusion protein as described herein for use in accordance with the present disclosure may be formulated into compositions (e.g., pharmaceutical compositions) according to known techniques. Vaccine preparation is generally described in Vaccine Design (Powell and Newman, 1995). For example, an immunologically amount of a vaccine product can be formulated together with one or more organic or inorganic, liquid or solid, pharmaceutically suitable carrier materials. Preparation of pneumococcal polysaccharide and conjugate vaccines is described, for example, in U.S. Ser. No. 11/395,593, filed Mar. 31, 2006, the contents of which are incorporated herein by reference.

In general, pharmaceutically acceptable carrier(s) include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (Martin, 1975).

Vaccines may be formulated by combining one or more fusion proteins described herein with carriers and/or other optional components by any available means including, for example, conventional mixing, granulating, dissolving, lyophilizing, or similar processes.

Vaccines comprising one or more fusion proteins described herein may be lyophilized up until they are about to be used, at which point they are extemporaneously reconstituted with diluent. In some embodiments, vaccine components or compositions are lyophilized in the presence of one or more other components (e.g., adjuvants), and are extemporaneously reconstituted with saline solution. Alternatively, individual components, or sets of components may be separately lyophilized and/or stored (e.g., in a vaccination kit), the components being reconstituted and either mixed prior to use or administered separately to the subject.

Lyophilization can produce a more stable composition (for instance by preventing or reducing breakdown of polysaccharide antigens). Lyophilizing of vaccines or vaccine components is well known in the art. Typically, a liquid vaccine or vaccine component is freeze dried, often in the presence of an anti-caking agent (such as, for example, sugars such as sucrose or lactose). In some embodiments, the anti-caking agent is present, for example, at an initial concentration of 10-200 mg/ml. Lyophilization typically occurs over a series of steps, for instance a cycle starting at −69° C., gradually adjusting to −24° C. over 3 h, then retaining this temperature for 18 h, then gradually adjusting to −16° C. over 1 h, then retaining this temperature for 6 h, then gradually adjusting to +34° C. over 3 h, and finally retaining this temperature over 9 h.

In some embodiments, a vaccine comprising a plurality of MAPS-X immunogenic complexes as described herein is a liquid. In some embodiments the liquid is a reconstituted lyophylate. In some embodiments a vaccine has a pH of about 5, about 6, about 7, or about 8. In some embodiments a vaccine has a pH between about 5 and about 7.5. In some embodiments a vaccine has a pH between 5 and 7.5. In some embodiments a vaccine has a pH between about 5.3 and about 6.3. In some embodiments a vaccine has a pH between 5.3 and 6.3. In some embodiments a vaccine has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

Vaccines or vaccine components for use in accordance with the present disclosure may be incorporated into liposomes, cochleates, biodegradable polymers such as poly-lactide, poly-glycolide and poly-lactide-co-glycolides, or immune-stimulating complexes (ISCOMS).

In certain situations, it may be desirable to prolong the effect or release of a vaccine for use in accordance with the present invention, for example, by slowing the absorption of one or more vaccine components. Such delay of absorption may be accomplished, for example, by the use of a liquid suspension of crystalline or amorphous material with poor water solubility. The rate of absorption of the product then depends upon its rate of dissolution, which in turn, may depend upon size and form. Alternatively, or additionally, delayed absorption may be accomplished by dissolving or suspending one or more vaccine components in an oil vehicle. Injectable depot forms can also be employed to delay absorption. Such depot forms can be prepared by forming microcapsule matrices of one or more vaccine components a biodegradable polymer network. Depending upon the ratio of polymer to vaccine component, and the nature of the particular polymer(s) employed, the rate of release can be controlled.

Examples of biodegradable polymers that can be employed in accordance with the present disclosure include, for example, poly(orthoesters) and poly(anhydrides). One particular exemplary polymer is polylactide-polyglycolide.

Depot injectable formulations may also be prepared by entrapping the product in liposomes or microemulsions, which are compatible with body tissues.

Polymeric delivery systems can also be employed in non-depot formulations including, for example, oral formulations. For example, biodegradable, biocompatible polymers such as ethylene vinyl acetate, polyanhydrides, polyglycolic acid, collagen, polyorthoesters, and polylactic acid, etc., can be used in oral formulations. Polysaccharide antigens or conjugates may be formulated with such polymers, for example to prepare particles, microparticles, extrudates, solid dispersions, admixtures, or other combinations in order to facilitate preparation of useful formulations (e.g., oral).

Vaccines comprising one or more fusion proteins described herein for use in accordance with the present disclosure include immunogenic compositions, and may additionally include one or more additional active agents (i.e., agents that exert a biological effect—not inert ingredients). For example, it is common in vaccine preparation to include one or more adjuvants. It will be appreciated that such additional agents may be formulated together with one or more other vaccine components, or may be maintained separately and combined at or near the time of administration. In some embodiments, such additional components may be administered separately from some or all of the other vaccine components, within an appropriate time window for the relevant effect to be achieved.

### (a) Adjuvants

The vaccine formulations and immunogenic compositions comprising a MAPS-X immunogenic complex and/or a fusion protein as described herein may include an adjuvant. Adjuvants, generally, are agents that enhance the immune response to an antigen. Adjuvants can be broadly separated into two classes, based on their principal mechanisms of action: vaccine delivery systems and immunostimulatory adjuvants (see, e.g., Singh et al, 2003). In most vaccine formulations, the adjuvant provides a signal to the immune system so that it generates a response to the antigen, and the antigen is required for driving the specificity of the response to the pathogen. Vaccine delivery systems are often particulate formulations, e.g., emulsions, microparticles, immune-stimulating complexes (ISCOMs), nanoparticles, which may be, for example, particles and/or matrices, and liposomes. In contrast, immunostimulatory adjuvants are sometimes from or derived from pathogens and can represent pathogen associated molecular patterns (PAMP), e.g., lipopolysaccharides (LPS), monophosphoryl lipid A (MPL), or CpG-containing DNA, which activate cells of the innate immune system.

Alternatively, adjuvants may be classified as organic and inorganic. Inorganic adjuvants include alum salts such as aluminum phosphate, amorphous aluminum hydroxyphosphate sulfate, and aluminum hydroxide, which are commonly used in human vaccines. Organic adjuvants comprise organic molecules including macromolecules. Non-limiting examples of organic adjuvants include cholera toxin/toxoids, other enterotoxins/toxoids or labile toxins/toxoids of Gram-negative bacteria, interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), and tumor necrosis factor (TNF).

Adjuvants may also be classified by the response they induce. In some embodiments, the adjuvant induces the generation, proliferation, or activation of Th1 cells or Th2 cells. In other embodiments, the adjuvant induces the generation, proliferation, or activation of B cells. In yet other embodiments, the adjuvant induces the activation of antigen-presenting cells. These categories are not mutually exclusive; in some cases, an adjuvant activates more than one type of cell.

In some embodiments, the adjuvant induces the generation, proliferation, or activation of Th17 cells. The adjuvant may promote the CD4+ or CD8+ T cells to secrete IL-17. In some embodiments, an adjuvant that induces the generation, proliferation, or activation of Th17 cells is one that produces at least a 2-fold, and in some cases a 10-fold, experimental sample to control ratio in the following assay. In the assay, an experimenter compares the IL-17 levels secreted by two populations of cells: (1) cells from animals immunized with the adjuvant and a polypeptide known to induce Th17 generation, proliferation, or activation, and (2) cells from animals treated with the adjuvant and an irrelevant (control) polypeptide. An adjuvant that induces the generation, proliferation, or activation of Th17 cells may cause the cells of population (1) to produce more than 2-fold, or more than 10-fold more IL-17 than the cells of population (2). IL-17 may be measured, for example, by ELISA or ELISPOT. Certain toxins, such as cholera toxin and labile toxin (produced by enterotoxigenic E. coli, or ETEC), activate a Th17 response. Thus, in some embodiments, the adjuvant is a toxin or toxoid. Cholera toxin was successfully used in a mouse model to induce protective immunity in conjunction with certain polypeptides from Table 1. One form of labile toxin is produced by Intercell. Mutant derivates of labile toxin (toxoids) that are active as adjuvants but significantly less toxic can be used as well. Exemplary detoxified mutant derivatives of labile toxin include mutants lacking ADP-ribosyltransferase activity. Particular detoxified mutant derivatives of labile toxin include LTK7 (Douce et al, 1995) and LTK63 (Williams et al, 2004), LT-G192 (Douce et al, 1999), and LTR72 (Giuliani et al, 1998).

In some embodiments, the adjuvant comprises a VLP (virus-like particle). One such adjuvant platform, Alphavirus replicons, induces the activation of Th17 cells using alphavirus and is produced by Alphavax. In some embodiments of the Alphavirus replicon system, alphavirus may be engineered to express an antigen of interest, a cytokine of interest (for example, IL-17 or a cytokine that stimulates IL-17 production), or both, and may be produced in a helper cell line. More detailed information may be found in U.S. Pat. Nos. 5,643,576 and 6,783,939. In some embodiments, a vaccine formulation is administered to a subject in combination with a nucleic acid encoding a cytokine.

Certain classes of adjuvants activate toll-like receptors (TLRs) in order to activate a Th17 response. TLRs are well known proteins that may be found on leukocyte membranes, and recognize foreign antigens (including microbial antigens). Administering a known TLR ligand together with an antigen of interest (for instance, as a fusion protein) can promote the development of an immune response specific to the antigen of interest. One exemplary adjuvant that activates TLRs comprises Monophosphoryl Lipid A (MPL). Traditionally, MPL has been produced as a detoxified lipopolysaccharide (LPS) endotoxin obtained from Gram-negative bacteria, such as S. minnesota. In particular, sequential acid and base hydrolysis of LPS produces an immunoactive lipid A fraction (which is MPL), and lacks the saccharide groups and all but one of the phosphates present in LPS. A number of synthetic TLR agonists (in particular, TLR-4 agonists) are disclosed in Evans et al, 2003. Like MPL adjuvants, these synthetic compounds activate the innate immune system via TLR. Another type of TLR agonist is a synthetic phospholipid dimer, for example E6020 (Ishizaka et al, 2007). Various TLR agonists (including TLR-4 agonists) have been produced and/or sold by, for example, the Infectious Disease Research Institute (IRDI), Corixa, Esai, Avanti Polar Lipids, Inc., and Sigma Aldrich. Another exemplary adjuvant that activates TLRs comprises a mixture of MPL, Trehalose Dicoynomycolate (TDM), and dioctadecyldimethylammonium bromide (DDA). Another TLR-activating adjuvant is R848 (resiquimod).

In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C.

In some embodiments, combinations of adjuvants are used. Three exemplary combinations of adjuvants are MPL and alum, E6020 and alum, and MPL and an ISCOM.

Adjuvants may be covalently or non-covalently bound to antigens. In some embodiments, the adjuvant may comprise a protein which induces inflammatory responses through activation of antigen-presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (e.g., see Wu et al, 2005).

In some embodiments, an immunogenic composition or vaccine comprising a plurality of MAPS-X immunogenic complexes and/or fusion protein as described herein is formulated and/or administered in combination with an adjuvant. In some embodiments, the adjuvant is selected from the group consisting of aluminum phosphate, aluminum hydroxide, and phosphate aluminum hydroxide. In some embodiments, the adjuvant comprises aluminum phosphate. In some embodiments, the adjuvant is aluminum phosphate.

In some embodiments, the vaccine composition as disclosed herein comprises an adjuvant. In some embodiments, the vaccine composition as disclosed herein comprises a R848 adjuvant. In some embodiments, the vaccine composition as disclosed herein comprises an ODN2395 adjuvant. In some embodiments, the vaccine composition as disclosed herein comprises an Alum phos adjuvant. In some embodiments, the vaccine composition as disclosed herein comprises an AddaSO3 alum adjuvant. In some embodiments, the vaccine composition as disclosed herein comprises a R848 alum adjuvant. In some embodiments, the vaccine composition as disclosed herein comprises an ODN alum adjuvant. In some embodiments, the vaccine composition as disclosed herein comprises an Alum 2PE adjuvant.

In some embodiments, the pharmaceutical composition as disclosed herein comprises an AddaSO3 adjuvant. In some embodiments, the pharmaceutical composition as disclosed herein comprises a R848 adjuvant. In some embodiments, the pharmaceutical composition as disclosed herein comprises an ODN2395 adjuvant. In some embodiments, the pharmaceutical composition as disclosed herein comprises an Alum phos adjuvant. In some embodiments, the pharmaceutical composition as disclosed herein comprises an AddaSO3 alum adjuvant. In some embodiments, the pharmaceutical composition as disclosed herein comprises a R848 alum adjuvant. In some embodiments, the pharmaceutical composition as disclosed herein comprises an ODN alum adjuvant. In some embodiments, the pharmaceutical composition as disclosed herein comprises an Alum 2PE adjuvant.

In some embodiments, the immunogenic composition as disclosed herein comprises an AddaSO3 adjuvant. In some embodiments, the immunogenic composition as disclosed herein comprises a R848 adjuvant. In some embodiments, the immunogenic composition as disclosed herein comprises an ODN2395 adjuvant. In some embodiments, the immunogenic composition as disclosed herein comprises an Alum phos adjuvant. In some embodiments, the immunogenic composition as disclosed herein comprises an AddaSO3 alum adjuvant. In some embodiments, the immunogenic composition as disclosed herein comprises a R848 alum adjuvant. In some embodiments, the immunogenic composition as disclosed herein comprises an ODN alum adjuvant. In some embodiments, the immunogenic composition as disclosed herein comprises an Alum 2PE adjuvant.

In some embodiments, the adjuvant can comprise of at least one of AddaSO3, R848, ODN2395, Alum phosphate, AddaSO3 alum, R848 alum, ODN alum, or Alum 2PE, at least two of AddaSO3, R848, ODN2395, Alum phosphate, AddaSO3 alum, R848 alum, ODN alum, or Alum 2PE, at least three of AddaSO3, R848, ODN2395, Alum phosphate, AddaSO3 alum, R848 alum, ODN alum, or Alum 2PE, at least four of AddaSO3, R848, ODN2395, Alum phosphate, AddaSO3 alum, R848 alum, ODN alum, or Alum 2PE, at least five of AddaSO3, R848, ODN2395, Alum phosphate, AddaSO3 alum, R848 alum, ODN alum, or Alum 2PE, at least six of AddaSO3, R848, ODN2395, Alum phosphate, AddaSO3 alum, R848 alum, ODN alum, or Alum 2PE, at least seven of AddaSO3, R848, ODN2395, Alum phosphate, AddaSO3 alum, R848 alum, ODN alum, or Alum 2PE, or at least eight of AddaSO3, R848, ODN2395, Alum phosphate, AddaSO3 alum, R848 alum, ODN alum, or Alum 2PE.

Typically, the same adjuvant or mixture of adjuvants is present in each dose of a vaccine. Optionally, however, an adjuvant may be administered with the first dose of vaccine and not with subsequent doses (i.e., booster shots). Alternatively, a strong adjuvant may be administered with the first dose of vaccine and a weaker adjuvant or lower dose of the strong adjuvant may be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human subjects, non-human animals, or both.

Vaccines for use in accordance with the present disclosure may include, or be administered concurrently with, antimicrobial therapy. For example, such vaccines may include or be administered with one or more agents that kills or retards growth of a pathogen. Such agents include, for example, penicillin, vancomycin, erythromycin, azithromycin, and clarithromycin, cefotaxime, ceftriaxone, levoflaxin, gatifloxacin.

Alternatively or additionally, vaccines for use in accordance with the present invention may include, or be administered with, one or more other vaccines or therapies.

### (b) Additional Components and Excipients

In addition to the fusion proteins described herein and the adjuvants described above, a vaccine formulation or immunogenic composition may include one or more additional components.

In some embodiments, the vaccine formulation comprises aluminum phosphate (referred to herein as alum phosphate, or AP). In some embodiments, a vaccine formulation comprising S. Paratyphi-MAPS aluminum phosphate (referred to herein as alum phosphate, or AP). In some embodiments, the amount of alum phosphate is determined by one of ordinary skill in the art. In some embodiments, the amount of alum phosphate is 250 g per 500 μl injection (25 g polysaccharide). In some embodiments, a vaccine formulation or immunogenic composition comprises 250 g of alum phosphate per 500 μl injection. In some embodiments, the alum phosphate is in a buffer comprising 20 mM Histadine, pH 6, 150 mM NaCl, 0.02% tween 80.

In some embodiments, the vaccine formulation or immunogenic composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In some embodiments, the vaccine formulation or immunogenic composition includes one or more buffers such as a mixture of sodium bicarbonate and ascorbic acid. In some embodiments, the vaccine formulation may be administered in saline, such as phosphate buffered saline (PBS), or distilled water.

In some embodiments, the vaccine formulation or immunogenic composition includes one or more surfactants, for example, but not limited to, polysorbate 80 (TWEEN 80), polysorbate 20 (TWEEN 20), Polyethylene glycol p-(1,1,3,3-tetramethylbutyl)-phenyl ether (TRITON X-100), and 4-(1,1,3,3-Tetramethylbutyl)phenol polymer with formaldehyde and oxirane (TYLOXAPOL). A surfactant can be ionic or nonionic.

In some embodiments, the vaccine formulation or immunogenic composition includes one or more salts such as sodium chloride, ammonium chloride, calcium chloride, or potassium chloride.

In some embodiments, a preservative is included in the vaccine or immunogenic composition. In other embodiments, no preservative is used. A preservative is most often used in multi-dose vaccine vials, and is less often needed in single-dose vaccine vials. In some embodiments, the preservative is 2-phenoxyethanol, methyl and propyl parabens, benzyl alcohol, and/or sorbic acid.

In another aspect of the invention, the MAPS-X immunogenic composition is lyophilized, optionally in the presence of at least one excipient. In one embodiment, the at least one excipient is selected from the group consisting of starch, glucose, lactose, sucrose, trehalose, raffinose, stachyose, melezitose, dextran, mannitol, lactitol, palatinit, gelatin, malt, rice, flour, chalk, silica gel, sodium stearate, glycerol monostearate, talc, glycine, arginine, lysine, sodium chloride (NaCl), dried skim milk, glycerol, propylene glycol, water, and ethanol. In a preferred embodiment, the at least one excipient is selected from the group consisting of sucrose, mannitol, and glycine. In a particular embodiment, the at least one excipient is sucrose. In one aspect, the lyophilized composition comprises about 1% (w/v) to about 10% (w/v) of the at least one excipient, preferably greater than about 5.5% (w/v). In another embodiment, the lyophilized composition comprises an additional excipient. In one such embodiment, the additional excipient is mannitol or glycine. In a preferred embodiment, the lyophilized composition comprises about 1% (w/v) to about 10% (w/v) of the additional excipient. In yet another embodiment, the lyophilized composition is reconstituted with water, water for injection (WFI), an adjuvant suspension, or saline. In a particular embodiment, the diluent is a suspension of any adjuvant described herein, such as an aluminum-based adjuvant suspension, preferably an aluminum phosphate suspension.

### XI. Methods of Administration

In some embodiments, an immunogenic composition or vaccine comprising a plurality of MAPS-X immunogenic complexes as described herein is administered to a subject at risk of developing an infection or disease from a pathogen, e.g. an infant, a toddler, a juvenile, or an older adult. In some embodiments, the immunogenic composition or vaccine is administered to a subject at elevated risk of developing or being infected with a pathogen, e.g., a pathogen from which the antigenic polysaccharide was derived, e.g., immunocompromised subjects, subjects having sickle cell disease or other hemoglobinopathies, congenital or acquired asplenia, splenic dysfunction, chronic renal failure or nephrotic syndrome, diseases associated with treatment with immunosuppressive drugs or radiation therapy, including malignant neoplasm, leukemia, lymphomas, Hodgkin's disease, or solid organ transplantation, congenital or acquired immunodeficiency, HIV infection, cerebrospinal fluid leaks, cochlear implant(s), chronic heart disease, chronic lung disease, diabetes mellitus, alcoholism, chronic liver disease, cigarette smoking, asthma, generalized malignancy, multiple myeloma, or solid organ transplantation. It will be appreciated that a subject can be considered at risk for developing a disease without having been diagnosed with any symptoms of the disease. For example, if the subject is known to have been, or to be intended to be, in situations with relatively high risk of infection, that subject will be considered at risk for developing the disease.

Any effective route of administration may be utilized such as, for example, oral, nasal, enteral, parenteral, intramuscular or intravenous, subcutaneous, transdermal, intradermal, rectal, vaginal, topical, ocular, pulmonary, or by contact application. In some embodiments, the immunogenic composition or vaccine may be injected (e.g., via intramuscular, intraperitoneal, intradermal and/or subcutaneous routes); or delivered via the mucosa (e.g., to the oral/alimentary, respiratory, and/or genitourinary tracts). Intranasal administration may be particularly useful in some contexts. In some embodiments, it may be desirable to administer different doses of the immunogenic composition or vaccine by different routes; in some embodiments, it may be desirable to administer different components of one dose via different routes.

In some embodiments, pharmaceutical compositions (e.g., immunogenic compositions or vaccines) are administered intradermally. Conventional technique of intradermal injection, the “Mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced while providing a slight pressure to elevate it under the skin. The liquid is then injected very slowly thereby forming a bleb or bump on the skin surface, followed by slow withdrawal of the needle.

Devices that are specifically designed to administer liquid agents into or across the skin have been described, for example the devices described in WO 99/34850 and EP 1092444, also the jet injection devices described for example in WO 01/13977; U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 97/37705 and WO 97/13537. Other methods of intradermal administration of the immunogenic compositions or vaccines may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

As described above, pharmaceutical compositions (e.g., immunogenic compositions or vaccines) may be administered as a single dose or as multiple doses. It will be appreciated that an administration is a single “dose” so long as all relevant components are administered to a subject within a window of time; it is not necessary that every component be present in a single composition. For example, administration of two different immunogenic compositions or vaccines, within a period of less than 24 h, is considered a single dose. To give but one example, immunogenic compositions or vaccines having different antigenic components may be administered in separate compositions, but as part of a single dose. As noted above, such separate compositions may be administered via different routes or via the same route. Alternatively or additionally, in embodiments wherein an immunogenic composition or vaccine is combined with additional types of active agents, the immunogenic composition or vaccine may be administered via one route, and a second active agent may be administered by the same route or by a different route.

Pharmaceutical compositions (e.g., immunogenic compositions or vaccines) are administered in such amounts and for such time as is necessary to achieve a desired result. In some embodiments of the present invention, the immunogenic composition or vaccine comprises an immunologically effective amount of at least immunogenic composition. The exact amount required to achieve an immunologically effective amount may vary, depending on the immunogenic composition, and from subject to subject, depending on the species, age, and general condition of the subject, the stage of the disease, the particular pharmaceutical mixture, its mode of administration, and the like.

The amount of a MAPS-X immunogenic complex and/or a fusion protein as described herein in each pharmaceutical composition (e.g., immunogenic composition or vaccine) dose is selected to allow the vaccine, when administered as described herein, to induce an appropriate immunoprotective response without significant adverse side effects.

In some embodiments, a pharmaceutical composition comprising a MAPS-X immunogenic complex and/or a fusion protein as described herein induces a Th1 and/or Th17 cell response upon administration to a subject. In some embodiments, the pharmaceutical composition induces an opsonic/bactericidal response against a pathogen of interest, e.g., a pathogen from which the antigenic polysaccharide was derived, upon administration to a subject. In some embodiments, the pharmaceutical composition comprising a MAPS-X immunogenic complex and/or a fusion protein disclosed herein reduces rate of transmission and/or colonization of the mucosal surfaces by a pathogen, e.g., a pathogen from which the antigenic polysaccharide was derived, upon administration to a subject. In some embodiments, the pharmaceutical composition reduces rate of transmission and/or colonization of the nasopharynx or the lungs by a pathogen, e.g., a pathogen from which the antigenic polysaccharide was derived, upon transmission.

Some embodiments provide for a method of immunizing a subject against infection of a pathogen, e.g., a pathogen from which the antigenic polysaccharide was derived, comprising administering to the subject an immunologically effective amount of an immunogenic composition comprising a MAPS-X immunogenic complex and/or a fusion protein described herein. Some embodiments provide for a method of immunizing a subject against infection by a pathogen, e.g., a pathogen from which the antigenic polysaccharide was derived, comprising administering to the subject an immunologically effective amount of a vaccine composition comprising a fusion protein described herein. Some embodiments provide for a method of immunizing a subject against infection by a pathogen, e.g., a pathogen from which the antigenic polysaccharide was derived, comprising administering to the subject an immunologically effective amount of a pharmaceutical composition comprising a fusion protein described herein.

### (a) Combination Prophylaxis or Combination Therapy

In some embodiments, an immunogenic composition or vaccine comprising a plurality of MAPS-X immunogenic complexes and/or fusion protein as described herein may be administered in combination with another agent. In some embodiments, the agent is or comprises PCV13. In some embodiments, the agent is or comprises PPSV23. In some embodiments, the agent is or comprises an antibiotic.

### (b) Dosing

In some embodiments, administration of an immunogenic composition or vaccine comprising a plurality of MAPS-X immunogenic complexes and/or fusion protein as described herein may involve the delivery of a single dose. In some embodiments, administration may involve an initial dose followed by one or several additional immunization doses, adequately spaced. An immunization schedule is a program for the administration of one or more specified doses of one or more specified MAPS-X vaccines, by one or more specified routes of administration, at one or more specified ages of a subject.

In some embodiments, administration of a vaccine (e.g., a vaccine composition) described herein may involve the delivery of a single dose. In some embodiments, administration may involve an initial dose followed by one or several additional immunization doses, adequately spaced. Such additional immunization doses can be referred to as boosters. In some embodiments, a booster (or second or subsequent) immunization dose is administered 2 weeks, or 3 weeks, or about 1 month, or about 2 months, or about 6 months or about 1 year after the preceding dose (where the proceeding dose can be initial dose or a second or third dose, or booster dose).

The present disclosure provides immunization methods that involve administering at least one dose of a vaccine to an infant subject. In some embodiments, the infant subject is 18 months old or younger. In some embodiments, the infant subject is 12 months old or younger. In some embodiments, the infant subject has previously received one or more doses of a MAPS-X vaccine; in other embodiments, the infant subject is naïve to a polysaccharide vaccine, including a MAPS or MAPS-X vaccines. In some embodiments, the infant subject has previously been infected with, or exposed to infection by a pathogen, e.g., a pathogen from which the antigenic polysaccharide was derived.

The present disclosure provides immunization methods that involve administering at least one dose of a vaccine to a toddler subject. In some embodiments, the toddler subject is 5 years old or younger. In some embodiments, the toddler subject is 4 years old or younger. In some embodiments, the toddler subject has previously received one or more doses of a polysaccharide vaccine; in other embodiments, the toddler subject is naïve to a MAPS or MAPS-X vaccines. In some embodiments, the toddler subject has previously been infected with, or exposed to infection by a pathogen, e.g., a pathogen from which the antigenic polysaccharide was derived.

The present disclosure provides immunization methods that involve administering at least one dose of a vaccine to a juvenile subject. In some embodiments, the juvenile subject is 18 years old or younger. In some embodiments, the juvenile subject is 15 years old or younger. In some embodiments, the juvenile subject has previously received one or more doses of a polysaccharide vaccine, including a MAPS or MAPS-X vaccine; in other embodiments, the juvenile subject is naïve to a MAPS or MAPS-X vaccines. In some embodiments, the juvenile subject has previously been infected with, or exposed to infection by a pathogen, e.g., a pathogen from which the antigenic polysaccharide was derived.

The present disclosure provides immunization methods that involve administering at least one dose of a vaccine to an adult subject. In some embodiments, the adult subject is older than about 50 years of age. In some embodiments, the adult subject is older than about 65 years of age. In some embodiments, the adult subject has previously received one or more doses of a polysaccharide vaccine, including a MAPS or MAPS-X vaccine; in other embodiments, the adult subject is naïve to a MAPS or MAPS-X vaccines. In some embodiments, the adult subject has previously been infected with, or exposed to infection by a pathogen, e.g., a pathogen from which the antigenic polysaccharide was derived.

Immunization schedules of the present disclosure are provided to induce an immune response (e.g., an immunoprotective response) in a subject sufficient to reduce at least one measure selected from the group consisting of incidence, prevalence, frequency, and/or severity of at least one infection, disease, or disorder, and/or at least one surrogate marker of the infection, disease, or disorder, in a population and/or subpopulation of the subject(s). A supplemental immunization schedule is one which has this effect relative to the standard schedule which it supplements. A supplemental schedule may call for additional administrations and/or supra-immunogenic doses of the immunogenic compositions or vaccines disclosed herein, found in the standard schedule, or for the administration of immunogenic compositions or vaccines not part of the standard schedule. A full immunization schedule of the present invention may comprise both a standard schedule and a supplemental schedule. Exemplary sample immunization schedules are provided for illustrative purposes. Detailed descriptions of methods to assess immunogenic response discussed herein allow one to develop alterations to the sample immunization schedules without undue experimentation.

In one embodiment of the present disclosure, a first administration of a MAPS-X vaccine usually occurs when a subject is more than about 2 weeks old, more than about 5 weeks old, more than about 1 year old, more than about 2 years old, more than about 15 years old, or more than about 18 years old.

In one embodiment of the present disclosure, a first administration of a MAPS-X vaccine usually occurs when a subject is more than about 50 years old, more than about 55 years old, more than about 60 years old, more than about 65 years old, or more than about 70 years old.

In some embodiments of the disclosure, a single administration of vaccine is employed. It is possible that the purposes of the present invention can be served with a single administration, especially when one or more utilized vaccine polypeptides, polysaccharide(s) and/or conjugate(s) or combinations thereof is/are strong, and in such a situation a single dose schedule is sufficient to induce a lasting immune-protective response.

In some embodiments, it is desirable to administer two or more doses of vaccine, for greater immune-protective efficacy and coverage. Thus, in some embodiments, a number of doses is at least two, at least three or more doses. There is no set maximum number of doses, however it is good clinical practice not to immunize more often than necessary to achieve the desired effect.

Without being bound by theory, a first dose of vaccine administered according to the disclosure may be considered a “priming” dose. In some embodiments, more than one dose is included in an immunization schedule. In such a scenario, a subsequent dose may be considered a “boosting” dose.

A priming dose may be administered to a naïve subject (a subject who has never previously received a conjugated polysaccharide vaccine). In some embodiments, a priming dose may be administered to a subject who has previously received conjugated polysaccharide vaccine at least five or more years previous to administration of an initial vaccine dose according to the invention. In other embodiments, a priming dose may be administered to a subject who has previously received a conjugated polysaccharide vaccine at least twenty or more years previous to administration of a priming vaccine according to the invention.

When an immunization schedule calls for two or more separate doses, the interval between doses is considered. The interval between two successive doses may be the same throughout an immunization schedule, or it may change as the subject ages. In immunization schedules of the present invention, once a first vaccine dose has been administered, there is a first interval before administration of a subsequent dose. A first interval is generally at least about 2 weeks, 1 month, 6 weeks, 2 months, 3 months, 6 months, 9 months, 12 months, or longer. Where more than one subsequent dose(s) are administered, second (or higher) intervals may be provided between such subsequent doses. In some embodiments, all intervals between subsequent doses are of the same length; in other embodiments, second intervals may vary in length. In some embodiments, the interval between subsequent doses may be at least about 12 months, at least about 15 months, at least about 18 months, at least about 21 months or at least about 2 years. In some embodiments, the interval between doses may be up to 3 years, up to about 4 years, or up to about 5 years or 10 years or more. In some embodiments, intervals between subsequent doses may decrease as the subject ages.

It will be appreciated by those skilled in the art that a variety of possible combinations and sub-combinations of the various conditions of timing of the first administration, shortest interval, largest interval and total number of administrations (in absolute terms, or within a stated period) exist, and all of these combinations and sub-combinations should be considered to be within the inventor's contemplation though not explicitly enumerated here.

### (c) Assays for Determining Immune Response

In some embodiments, a method of assessing the immunogenicity of a pharmaceutical composition, immunogenic composition, or vaccine comprising a plurality of MAPS-X immunogenic complexes as described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), agglutination, motility, cytotoxicity, or adherence; and in vivo assays in animal models of infections by a pathogen of interest, e.g., a pathogen from which the antigenic polysaccharide was derived. Animal models of infection for S. pneumococcus and Streptococcus agalactiae disease are well known (e.g. pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with a pathogen of interest that are the targets of the immunogenic composition, e.g., a pathogen from which the antigenic polysaccharide was derived. In some embodiments, the immune response is compared to a control composition, including but not limited to vaccines comprising polysaccharides of a pathogen that are not cross-linked, and/or MAPS compositions not comprising a SBD-BBM fusion protein.

In some embodiments, a method of assessing the potency of a pharmaceutical composition, immunogenic composition, or vaccine comprising a MAPS-X immunogenic complex as described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), internalization, activity neutralization, agglutination, motility, cytotoxicity, or adherence; and in vivo assays in animal models of the pathogen which is the target of the MAPS-X immunogenic complex. Animal models of infection for S. pneumococcus and Streptococcus agalactiae disease are well known (e.g. pneumonia, bacteremia, meningitis, sepsis, otitis media, nasopharyngeal colonization). Parameters include bacterial clearance or reduction from mucosal surfaces or bloodstream, reduction or prevention of bacteremia, meningitis, sepsis, or otitis media, reduction or prevention of colonization of the nasopharynx, reduction of mortality, and passive and active protection following challenge with a pathogen of interest that is the target of the immunogenic composition, e.g., a pathogen from which the antigenic polysaccharide was derived. In some embodiments, the immune response is compared to a control composition.

Generally speaking, it may be desirable to assess humoral responses, cellular responses, and/or interactions between the two. Where humoral responses are being assessed, antibody titers and/or types (e.g., total IgG, IgG1, IgG2, IgM, IgA, etc.) to specific pathogen antigens (e.g., polypeptides or polysaccharides, either serotype-specific or conserved across two or more serotypes) may be determined, for example before and/or after administration of an initial or a boosting dose of vaccine (and/or as compared with antibody levels in the absence of antigenic stimulation). Cellular responses may be assessed by monitoring reactions such as delayed type hypersensitivity responses, etc. to the antigens. Cellular responses can also be measured directly by evaluating the response of peripheral blood mononuclear cells (PBMCs) monocytes to stimulation with the antigens of interest. Precursor and memory B cell populations may be assessed in enzyme-linked immunospot (ELISpot) assays directed against specific pathogen antigens.

The RIA method detects specific antibodies through incubation of sera with radio-labeled polysaccharides or polypeptides in suspension (e.g., Schiffiman et al, 1980). The antigen-antibody complexes are then precipitated with ammonium sulfate and the radiolabeled pellets assayed for counts per minute (cpm).

In the ELISA detection method, specific antibodies from the sera of vaccinated subjects are quantitated by incubation with antigens (e.g., polypeptides or polysaccharides, either serotype-specific or conserved across two or more serotypes) which have been adsorbed to a solid support (e.g., Koskela and Leinonen (1981); Kojima et al, 1990; Concepcion and Frasch, 2001). The bound antibody is detected using enzyme-conjugated secondary detection antibodies. The ELISA also allows isotyping and subclassing of the immune response (i.e., IgM vs. IgG or IgG1 vs. IgG2) by using isotype- or subclass-specific secondary antibodies and can be adapted to evaluate the avidity of the antibodies (Anttila et al, 1998; Romero-Steiner et al, 2005). Multiplex assays (e.g., Luminex) facilitate simultaneous detection of antibodies to multiple antigens. Antigens are conjugated to spectrally distinct microspheres that are mixed and incubated with serum. The antibodies bound to the antigens on the coated microspheres are detected using a secondary antibody (e.g., R-Phycoerythrin-conjugated goat anti-human IgG).

An approach for assessing functional antibody in serum is the opsonophagocytic assay (OPA) which quantitates only the antibodies that can opsonize the bacteria, leading to ingestion and killing of the bacteria. The standard assay utilizes a human phagocytic effector cell, a source of complement, bacteria, and diluted sera. The assay readout is the serum endpoint titer at which there is >50% killing compared to bacteria incubated with complement and human cells alone (Romero-Steiner et al, 1997). This killing OPA can also be multiplexed by utilizing target strains of pathogen that carry different antibiotic resistance markers (Kim et al, 2003). Another type of multiplex opsonic assay is a nonkilling assay in which the uptake by phagocytic effector cells of fluorescent stained encapsulated pathogen or fluorescent microspheres conjugated with antigens from a target pathogen in the presence of diluted sera plus a complement source is evaluated by FC (Martinez et al, 1999). Opsonic activity of serum antibody plus complement can also be evaluated by measuring the oxidative response of phagocytic human effector cells to ingested pathogen (Munro et al. 1985; Ojo-Amaize et al. 1995).

Certain in vivo model systems can be used to evaluate the protection afforded by serum antibodies induced by immunogenic compositions or vaccines comprising a fusion protein described herein. In such passive protection systems, mice or rats are challenged with the pathogen plus diluted sera, and the endpoint titer of the sera which provides protection against pneumonia, bacteremia, colonization of organs or tissues, or mortality is determined (Stack et al. 1998; Saeland et al. 2000).

In some embodiments, efficacy of immunization may be determined by assaying one or more cytokine levels by stimulating T cells from a subject after immunization. The one or more cytokine levels may be compared to the one or more cytokine levels in the same subject before immunization. Increased levels of the one or more cytokine, such as a 1.5 fold, 2-fold, 5-fold, 10-fold, 20-fold, 50-fold or 100-fold or more increase over pre-immunization cytokine levels, would indicate an increased response to the immunogenic composition or vaccine. In some embodiments, the one or more cytokines are selected from GM-CSP; IL-1a; IL-10; IL-2; IL-3; IL-4; IL-5; IL-6; IL-7; IL-8; IL-10; IL-12; IL-17A, IL-17F or other members of the IL-17 family; IL-22; IL-23; IFN-α; IFN-β; IFN-γ; MIP-1α; MIP-1β; TGF-β; TNFα, or TNF-β. In a non-limiting example, efficacy of immunization may be determined by assaying IL-17 levels (particularly IL-17A) by stimulating T cells from a subject after immunization. The IL-17 levels may be compared to IL-17 levels in the same subject before immunization. Increased IL-17 (e.g., IL-17A) levels, such as a 1.5 fold, 2-fold, 5-fold, 10-fold, 20-fold, 50-fold or 100-fold or more increase, would indicate an increased response to the immunogenic composition or vaccine.

In some embodiments, one may assay neutrophils in the presence of T cells or antibodies from the patient for pathogenic killing. Increased killing by the pathogen of interest, e.g., a pathogen from which the antigenic polysaccharide was derived, such as a 1.5 fold, 2-fold, 5-fold, 10-fold, 20-fold, 50-fold or 100-fold or more increase, would indicate an increased response to the MAPS-X immunogenic complex or vaccine. For example, one may measure Th17 cell activation, where increased Th17 cell activation, such as a 1.5 fold, 2-fold, 5-fold, 10-fold, 20-fold, 50-fold or 100-fold or more increase, correlates with an increased response to the immunogenic composition or vaccine. In another non-limiting example, one may measure Th1 cell activation, where increased Th1 cell activation, such as a 1.5 fold, 2-fold, 5-fold, 10-fold, 20-fold, 50-fold or 100-fold or more increase, correlates with an increased response to the MAPS-X immunogenic complex or vaccine. One may also measure levels of an antibody specific to the immunogenic composition or vaccine, where increased levels of the specific antibody, such as a 1.5 fold, 2-fold, 5-fold, 10-fold, 20-fold, 50-fold or 100-fold or more increase, are correlated with increased efficacy. In some embodiments, two or more of these assays are used. For example, one may measure IL-17 levels and the levels of immunogenic composition- or vaccine-specific antibody. Alternatively, one may follow epidemiological markers such as incidence of, severity of, or duration of infection with the pathogen of interest, e.g., a pathogen from which the antigenic polysaccharide was derived, in vaccinated individuals compared to unvaccinated individuals.

Immunogenic composition or vaccine efficacy may also be assayed in various model systems such as the mouse challenge model. For instance, BALB/c or C57BL/6 strains of mice may be used. After administering the test MAPS-X immunogenic complex or vaccine to a subject (as a single dose or multiple doses), the experimenter administers a challenge dose of the pathogen of interest. In some cases, a challenge dose administered intranasally is sufficient to cause colonization (especially nasal colonization) by the pathogen of interest, e.g., a pathogen from which the antigenic polysaccharide was derived, in an unvaccinated animal, and in some cases a challenge dose administered via aspiration is sufficient to cause sepsis and a high rate of lethality in unvaccinated animals. In some cases, a challenge dose administered via intraperitoneal injection is sufficient to cause sepsis and a high rate of lethality in unvaccinated animals. In some cases, a challenge dose administered via intravenous injection is sufficient to cause sepsis and a high rate of lethality in unvaccinated animals. One can then measure the reduction in colonization or the reduction in lethality in vaccinated animals.

Certain in vivo model systems can be used to evaluate the protection afforded by serum antibodies induced by vaccines of the present invention. In such passive protection systems, mice or rats are challenged with the pathogen plus diluted sera, and the endpoint titer of the sera which provides protection against bacteremia, colonization of organs or tissues, or mortality is determined (Stack et al. 1998; Saeland et al. 2000).

Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the pneumococcal pathogens that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the potency of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), internalization, activity neutralization, agglutination, motility, cytotoxicity, or adherence; and in vivo assays in animal models of a pathogen of interest, for example, if the pathogen is a S. pneumococcus, an animal model of pneumococcal disease (e.g. pneumonia, bacteremia, meningitis, sepsis, otitis media, nasopharyngeal colonization) can be used. Parameters of in vivo assays include bacterial clearance or reduction from mucosal surfaces or bloodstream, reduction or prevention of bacteremia, meningitis, sepsis, or otitis media, reduction or prevention of colonization of the nasopharynx, reduction of mortality, and passive and active protection following challenge with the pneumococcal pathogens that are the targets of the immunogenic composition. In some embodiments, the immune response is compared to a control composition. In some embodiments, a control composition may comprise an antigenic polysaccharide present in the immunogenic composition but not comprise a SBD-BBM fusion protein as disclosed herein, or not comprise an antigenic polypeptide present in the MAPS-X immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the MAPS-X immunogenic composition.

In some embodiments, a method of assessing the immunogenicity of a MAPS-X vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, Th1/Th17 cell response, cytokine level measurement and functional antibody levels as measured by OPK, serum bactericidal killing (SBA), agglutination, motility, cytotoxicity, or adherence; and in vivo assays in animal models of a pathogen of interest, e.g., a pathogen from which the antigenic polysaccharide was derived. In some embodiments, an animal disease model is based on a model of pneumococcal disease (e.g. pneumonia, bacteremia, meningitis, sepsis, otitis media, nasopharyngeal colonization). Parameters of in vivo assays include bacterial clearance from mucosal surfaces or bloodstream, reduction or prevention of bacteremia, meningitis, sepsis, or otitis media, reduction or prevention of colonization of the nasopharynx, reduction of mortality, and passive and active protection following challenge with the pneumococcal pathogens that are the targets of the immunogenic composition. In some embodiments, the immune response is compared to a control composition. In some embodiments, a control composition may comprise an antigenic polysaccharide present in the immunogenic composition but not comprise a SBD-BBM fusion protein as disclosed herein, or not comprise an antigenic polypeptide present in the MAPS-X immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the MAPS-X immunogenic composition.

### X. Manufacture of Immunogenic Complexes

In some embodiments, SBD-BBM fusion proteins as described herein may be non-covalently associated with antigenic polysaccharides as disclosed herein, by biotin to biotin-binding protein interaction, e.g., biotin to rhizavidin protein interaction, and/or by SBD interaction with sialic acid on the antigenic polysaccharide or van de Waals interactions with positively charged polysaccharides, as disclosed herein. In some embodiments, the polysaccharide is a purified antigenic polysaccharide, including a purified lipidated capsular polysaccharide oligosaccharide.

In some embodiments, a SBD-BBM fusion protein as described herein is covalently bound to another molecule. This may, for example, increase the half-life, solubility, bioavailability, or immunogenicity of the fusion protein. Molecules that may be covalently bound to the fusion protein include a carbohydrate, biotin, poly(ethylene glycol) (PEG), polysialic acid, N-propanoylated polysialic acid, nucleic acids, polysaccharides, and PLGA. There are many different types of PEG, ranging from molecular weights of below 300 g/mol to over 10,000,000 g/mol. PEG chains can be linear, branched, or with comb or star geometries. In some embodiments, the fusion protein is covalently bound to a moeity that stimulates the immune system. An example of such a moeity is a lipid moeity. In some instances, lipid moieties are recognized by a Toll-like receptor (TLR) such as TLR-2 or TLR-4, and activate the innate immune system.

The present disclosure includes methods for manufacturing MAPS-X immunogenic complexes described herein. In some embodiments, a method of manufacturing MAPS-X immunogenic complexes comprises complexing at least one biotinylated polysaccharide with at least one fusion protein as disclosed herein, e.g., a SBD-BBM fusion protein, and/or a SBD-[Ag]-BBM fusion protein as disclosed herein.

In some embodiments, a fusion protein e.g., a SBD-[Ag]-BBM fusion protein and one or more additional components described herein are mixed together using known methods to form a multi-component immunogenic composition. In some embodiments, a fusion protein and one or more additional components described herein are nano-encapsulated using known methods. In some embodiments, a fusion protein and one or more additional components described herein are molded into nano- or micro-particles using known methods. In some embodiments, a fusion protein and one or more additional components described herein are conjugated through a covalent bond using known methods to form a multi-component immunogenic composition. In some embodiments, a fusion protein e.g., a SBD-[Ag]-BBM fusion protein and one or more additional components described herein are joined non-covalently using known methods to form a multi-component immunogenic composition. Additional methods of combining a fusion protein and one or more additional components are described in, e.g., PCT/US20I2/374I2 and PCT/US2009/44956.

In some embodiments, the average (e.g., the mean) protein (e.g., antigenic protein) to polysaccharide ratio of a plurality of MAPS-X immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average protein to polysaccharide ratio of a plurality of MAPS-X immunogenic complexes is approximately 1:1 (w/w). In some embodiments, the average protein to polysaccharide ratio of a plurality of MAPS-X immunogenic complexes is approximately 2:1 (w/w). In some embodiments, the average protein to polysaccharide ratio of a plurality of MAPS-X immunogenic complexes is approximately 3:1 (w/w). In some embodiments, the average protein to polysaccharide ratio of a plurality of MAPS-X immunogenic complexes is approximately 4:1 (w/w). In some embodiments, the average protein to polysaccharide ratio of a plurality of MAPS-X immunogenic complexes is approximately 5:1 (w/w). In some embodiments, the average protein to polysaccharide ratio of a plurality of MAPS-X immunogenic complexes is approximately 6:1 (w/w). In some embodiments, the average protein to polysaccharide ratio of a plurality of MAPS-X immunogenic complexes is approximately 7:1 (w/w). In some embodiments, the average protein to polysaccharide ratio of a plurality of MAPS-X immunogenic complexes is approximately 8:1 (w/w). In some embodiments, the average protein to polysaccharide ratio of a plurality of MAPS-X immunogenic complexes is approximately 9:1 (w/w). In some embodiments, the average protein to polysaccharide ratio of a plurality of MAPS-X immunogenic complexes is approximately 10:1 (w/w). In some embodiments, the average protein:PS ratios are chosen to enhance the polysaccharide immunogenicity potential (carrier function) and/or to elicit protection against, or to inhibit, colonization by the pathogen of interest (independent of polysaccharide serotype) through a protein-specific immune response. Immunogenic compositions and vaccines of the invention may comprise mixtures of MAPS-X immunogenic complexes with different average protein to polysaccharide ratios.

In some embodiments, a vaccine or immunogenic composition comprises a plurality of MAPS-X immunogenic complexes comprising any one or more of SBD-BBM fusion proteins as disclosed. In some embodiments, the average ratio of a SBD-BBM fusion protein as disclosed herein to am antigenic polysaccharide from a pathogen of interest, in the plurality of MAPS-X immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of total fusion protein of a SBD-BBM fusion protein as disclosed herein, to a biotinylated polysaccharide from or derived from any pathogen of interest, in the plurality of MAPS-X immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of total fusion protein selected from any of: (i) a SBD-BBM fusion protein, or (ii) SBD-[Ag]-BBM fusion protein, (iii) or a SBD-[Ag] fusion protein, or (iv) BBM-[Ag] fusion protein as disclosed herein, to a biotinylated polysaccharide from or derived from any pathogen of interest, in the plurality of MAPS-X immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]).

In some embodiments, the average ratio of total fusion protein of a SBD-BBM fusion protein as disclosed herein to a polysaccharide from or derived from a pathogen of interest in the plurality of MAPS-X immunogenic complexes is chosen to enhance the polysaccharide immunogenicity potential and/or to elicit protection against, or to inhibit, pathogenic colonization by any subtype of the pathogen (independent of polysaccharide serotype) through a protein specific immune response. Immunogenic compositions and vaccines of the invention may comprise mixtures of MAPS-X immunogenic complexes with different average protein to polysaccharide ratios.

### XI. Kits

The present disclosure also provides for kits for producing a MAPS-X immunogenic complex as disclosed herein which is useful for an investigator to tailor a MAPS-X immunogenic complex with their preferred polysaccharide antigens and/or polypeptide antigens, e.g., for research purposes to assess the effect of a polysaccharide antigen and/or polypeptide antigen, or a combination of antigens on immune response. Such kits can be prepared from readily available materials and reagents. For example, such kits can comprise any one or more of the following materials: a container comprising a polysaccharide cross-linked with a plurality of biotin or biotin like molecules and a container comprising a SBD-BBM fusion protein.

In another embodiment, the kit comprises a container comprising a biotinylated polysaccharide, e.g., a polysaccharide of interest, and optionally a container comprising control polysaccharide; a container comprising biotin, container comprising at least one fusion protein as disclosed herein, e.g., a SBD-BBM fusion protein; and a container comprising a cross-linking reagent for cross-linking a biotin to the polysaccharide, for example, but not limited to, CDAP (1-cyano-4-dimethylaminopyridinium tetrafluoroborate), and EDC (1-Ethyl-3-[3-dimethylaminopropyl]carbodiimide hydrochloride).

In another embodiment, the kit comprises a container comprising one or more fusion protein e.g., a SBD-BBM fusion protein and optionally, a container comprising a SBD-[Ag]-BBM fusion protein, and/or alternatively, a vector comprising a nucleic acid encoding either (i) SBD-BBM fusion protein and/or a vector comprising a nucleic acid encoding a SBD-[Ag]-BBM fusion protein as disclosed herein, which can optionally comprise a cloning site of inserting a nucleic acid encoding an antigen of interest.

In some embodiments, the kit can comprise at least one co-stimulation factor which can be added to the polysaccharide. In some embodiments, the kit comprises a cross-linking reagent, for example, but not limited to, CDAP (1-cyano-4-dimethylaminopyridinium tetrafluoroborate); EDC (1-Ethyl-3-[3-dimethylaminopropyl]carbodiimide hydrochloride); sodium cyanoborohydride; cyanogen bromide; and ammonium bicarbonate/iodoacetic acid, for linking the co-factor to the polysaccharide.

A variety of kits and components can be prepared for use in the methods described herein, depending upon the intended use of the kit, the particular target antigen and the needs of the user.

In some embodiments, the present invention may be defined in any of the following numbered paragraphs:
1. An immune composition comprising one or more species of MAPS-X immunogenic complexes, wherein each species of the MAPS-X immunogenic complex comprises: (a) at least a first biotinylated antigen polysaccharide antigen (PS1) from a pathogen, (b) at least a second biotinylated polysaccharide (PS2) from a pathogen, (c) at least a first fusion protein comprising, or consisting of: a biotin-biotin moiety (BBM) and a sialic acid binding moiety (SBD) (SBD-BBM fusion protein), wherein the BBM of the at least first fusion protein non-covalently associates with at least one biotin molecule on the first biotinylated antigenic polysaccharide (PS1), and the SBD non-covalently associates with the second antigenic polysaccharide, or wherein a SBD of the at least first fusion protein non-covalently associates with the first biotinylated antigenic polysaccharide and the BBM non-covalently associates with at least biotin molecule on the second biotinylated antigenic polysaccharide, and wherein the first biotinylated antigenic polysaccharide and the second biotinylated antigenic polysaccharide are indirectly joined by their non-covalent association with the first fusion protein to form a MAPS-X immunogenic complex.
2. The immune composition of paragraph 1, wherein the PS1 and PS2 are located on the same polysaccharide macromolecule, thereby enabling the SBD-BBM to join two polysaccharide antigens within the same polysaccharide polymer macromolecule.
3. The immune composition of paragraph 1, wherein the PS1 and PS2 are located on distinct polysaccharide macromolecules, thereby enabling the SBD-BBM to join two polysaccharide antigens from two or more distinct polysaccharide polymer macromolecules.
4. The immune composition of paragraph 3, wherein one polysaccharide macromolecule is from a pathogen, and the other macromolecule is from a distinct pathogen or serotype.
5. The immune composition of any of paragraphs 1-4, wherein the first or second biotinylated antigenic polysaccharide, or both, comprise a sialic acid molecule.
6. The immune composition of any of paragraphs 1-5, wherein the first or second biotinylated antigenic polysaccharide, or both, do not comprise a sialic acid molecule.
7. The immune composition of any of paragraphs 1-6, wherein the SBD of the first fusion protein associates with sialic acid on the first or second antigenic polysaccharide, and/or a negatively charged first or second antigenic polysaccharide.
8. The immune composition of any of paragraph s 1-8, wherein each MAPS-X immunogenic complex further comprises at least a second fusion protein, wherein if the BBM of the at least first fusion protein non-covalently associates with at least one biotin molecule on the first biotinylated antigenic polysaccharide (PS1), then the BBM of the at least second fusion protein non-covalently associates with at least one biotin molecule on the second biotinylated antigenic polysaccharide (PS2), and the SBD non-covalently associates with the first antigenic polysaccharide (PS1), or
wherein if the BBM of the at least first fusion protein non-covalently associates with at least one biotin molecule on the second biotinylated antigenic polysaccharide (PS2), then the BBM of the at least second fusion protein non-covalently associates with at least one biotin molecule on the first biotinylated antigenic polysaccharide (PS1), and the SBD non-covalently associates with the second antigenic polysaccharide (PS2).
9. The immune composition of any of paragraphs 1-8, wherein the MAPS-X immunogenic complex further comprises (i) a plurality of first biotinylated antigenic polysaccharides, and (ii) a plurality of second antigenic polysaccharides, and (ii) a plurality of first fusion proteins and a plurality of second fusion proteins, wherein each first biotinylated antigenic polysaccharide can be indirectly joined to more than one second antigenic polysaccharides by more than one first or second fusion protein, and vice versa, each second biotinylated antigenic polysaccharide can be indirectly joined to more than one first antigenic polysaccharides by more than one first or second fusion protein.
10. The immune composition of any of paragraphs 1-9, wherein the first antigenic polysaccharide and the second antigenic polysaccharide in a species MAPS-X immunogenic complex are from the same pathogenic organism.
11. The immune composition of any of paragraphs 1-10, wherein the first antigenic polysaccharide and the second antigenic polysaccharide in a species of MAPS-X immunogenic complex are from a different pathogenic organism.
12. The immune composition of any of paragraphs 1-11, wherein the first antigenic polysaccharide and the second antigenic polysaccharide in of MAPS-X immunogenic complex are from the same subtype of pathogenic organism, or from a different subtype of a pathogenic organism.
13. The immune composition of any of paragraphs 1-12, comprising: (a) two or more species of MAPS-X immunogenic complexes, each species of MAPS-X immunogenic complex comprises a first antigenic polysaccharide and second antigenic polysaccharide that is from a distinct pathogenic organism, and/or (b) two or more species of MAPS-X immunogenic complexes, each species of MAPS-X immunogenic complex comprises a first antigenic polysaccharide and second antigenic polysaccharide that is from a distinct subtype or serotype of the same pathogenic organism.
14. The immune composition of any of paragraphs 1-13, wherein the immune composition comprises at least 2, or at least 3, or at least 4, or at least 5 or more than 5 distinct species of MAPS-X immunogenic complexes.
15. The immune composition of any of paragraphs 1-14, wherein the immune composition comprises at least 6-10, 11-15, 16-20, 21-25, 26-30, or 31-35 or more than 35 distinct species of MAPS-X immunogenic complexes.
16. The immune composition of any of paragraphs 1-15, wherein the first or second fusion protein further comprise at least a one polypeptide antigen or an antigenic fragment thereof, where the biotin binding moiety (BBM), sialic acid binding moiety (SBD), and at least one polypeptide antigen or an antigenic fragment thereof can be in any order.
17. The immune composition of any of paragraphs 1-16, wherein each species of MAPS-X immunogenic complex further comprises: (a) a third fusion protein comprising, in any order, a biotin binding moiety (BBM), and at least one polypeptide antigen or an antigenic fragment thereof, and/or (b) a fourth fusion protein comprising, in any order, a sialic acid binding moiety (SBD), and at least one polypeptide antigen or an antigenic fragment thereof.
18. The immune composition of any of paragraphs 1-17, wherein the BBM is Rhizavidin and comprises an amino acid sequence having at least 80%, or at least 85%, or at least 90%, or at least 95% sequence identity to SEQ ID NO: 1 or SEQ ID NO: 2.
19. The immune composition of any of paragraphs 1-18, wherein SBD is selected from any of: SBD1, SBD2, SBD3, SBD4, NanH, NanH2, NanH3, VcNanH, or a SBD comprising an amino acid sequence that at least 80%, or at least 85%, or at least 90%, or at least 95% sequence identity to any of: SEQ ID NO: 3-12.
20. The immune composition of any of paragraphs 1-19, wherein the first or second fusion protein comprises: (a) an amino acid sequence having at least 80%, or at least 85%, or at least 90%, or at least 95% sequence identity to SEQ ID NO: 1 or SEQ ID NO: 2 (Rhavi), and (b) a SBD polypeptide, where the SBD polypeptide selected from any of: (i) an amino acid sequence having at least 80%, or at least 85%, or at least 90%, or at least 95% sequence identity to SEQ ID NO: 3 (SBD1), (ii) an amino acid sequence having at least 80%, or at least 85%, or at least 90%, or at least 95% sequence identity to SEQ ID NO: 4 (SBD2), (iii) an amino acid sequence having at least 80%, or at least 85%, or at least 90%, or at least 95% sequence identity to SEQ ID NO: 5 (SBD3), (iv) an amino acid sequence having at least 80%, or at least 85%, or at least 90%, or at least 95% sequence identity to SEQ ID NO: 6 (SBD4), (v) an amino acid sequence having at least 80%, or at least 85%, or at least 90%, or at least 95% sequence identity to SEQ ID NO: 7 (NanH), (vi) an amino acid sequence having at least 80%, or at least 85%, or at least 90%, or at least 95% sequence identity to SEQ ID NO: 8 (NanH2), and (vii) an amino acid sequence having at least 80%, or at least 85%, or at least 90%, or at least 95% sequence identity to SEQ ID NO: 9 (NanH3).
21. The immune composition of any of paragraphs 1-20, wherein the first or second fusion protein is selected from a fusion protein comprising an amino acid sequence having at least 80%, or at least 85%, or at least 90%, or at least 95% sequence identity to any of: SEQ ID NO: 9-20.
22. The immunogenic composition of any of paragraphs 1-22, wherein the first or second fusion protein comprises at least one linker sequence or at least one spacer sequence located between any of: the BBM and the SBD; the BBM and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD.
23. The immunogenic composition of any of paragraphs 1-22, wherein the linker sequence or spacer sequence is selected from any of: GGGGSSS, TDPNSSS, SSS, AAA.
24. The immunogenic composition of any of paragraphs 1-23, wherein the polypeptide antigen or fragment thereof is from the same pathogen as the first or second, or both polysaccharides.
25. The immunogenic composition of any of paragraphs 1-24, wherein the polypeptide antigen or fragment thereof is from a different pathogen to the pathogen of the first or second polysaccharides.
26. The immunogenic composition of any of paragraphs 1-25, wherein the first or second polysaccharide, or both, is selected from the group consisting of: polysaccharides, oligosaccharides, or lipopolysaccharides from Gram-positive bacteria; polysaccharides, oligosaccharides, or lipopolysaccharides from Gram-negative bacteria; other bacterial capsular or cell wall polysaccharides; fungal polysaccharides; viral polysaccharides; and polysaccharides derived from cancer or tumor cells.
27. The immunogenic composition of any of paragraphs 1-26, wherein the first or second antigenic polysaccharide, or both, are selected from the group consisting of: Salmonella typhi Vi capsular polysaccharides; Salmonella polysaccharides; Shigella polysaccharide, pneumococcal polysaccharides; Haemophili polysaccharides; Meningococcal polysaccharides; Staphylococcus aureus polysaccharides; Bacillus anthracis polysaccharides; Streptococcus polysaccharides; Pseudomonas polysaccharides; Cryptococcus polysaccharides; and viral glycoproteins.
28. The immunogenic composition of any of paragraphs 1-27, wherein the first or second antigenic polysaccharide, or both, are pneumococcal capsular polysaccharides.
29. The immunogenic composition of paragraph 28, comprising at least 15 or more species of MAPS-X immunogenic composition, wherein each species of MAPS-X immunogenic complex comprises a first antigenic polysaccharide and second antigenic polysaccharide that is from a distinct subtype of Streptococcus pneumoniae.
30. The immunogenic composition of paragraph 25, wherein the first and second immunogenic polysaccharide for each species of MAPS-X immunogenic complex are selected from Streptococcus pneumoniae serotypes: 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.
31. The immune composition of any of paragraphs 1-30, wherein the first or second antigenic polysaccharide, or both, is a capsular polysaccharide (CP) from a Group B Streptococcus (GBS) or Streptococcus agalactiae.
32. The immunogenic composition of paragraph 31, wherein the first or second immunogenic polysaccharide, or both for each species of MAPS-X immunogenic complex are selected from Streptococcus agalactiae serotypes Ia, Ib, II, III, IV, V, VI, VII, VIII, and IX, 33. The immune composition of any of paragraphs 1-32, wherein the first or second antigenic polysaccharide, or both, is not from a Group B Streptococcus (GBS) or Streptococcus agalactiae.
34. The immune composition of any of paragraphs 1-33, wherein the first biotinylated polysaccharide, or second biotinylated polysaccharide, or both, has a sialic acid level of greater than about 20% or about 30%, or about 40%, or about 50% or about 60%.
35. A pharmaceutical composition comprising the immunogenic composition of any of paragraphs 1-34, and a pharmaceutically acceptable carrier.
36. The pharmaceutical composition of paragraph 35, further comprising one or more adjuvants.
37. The pharmaceutical composition of paragraph 36, wherein the one or more adjuvants is or comprises a co-stimulation factor.
38. The pharmaceutical composition of paragraph 35, wherein the one or more adjuvants are selected from the group consisting of aluminum phosphate, aluminum hydroxide, and phosphated aluminum hydroxide.
39. A vaccine comprising the immunogenic composition of any of paragraphs 1-34 and a pharmaceutically acceptable carrier.
40. A method of making a multivalent vaccine, comprising mixing four or more species of MAPS-X immunogenic complexes of any of paragraphs 1-34 in a single formulation.
41. The method of paragraph 40, comprising mixing four or more species of MAPS-X immunogenic complexes in a single formulation.
42. Use of immune composition of any of paragraphs 1-34, or the vaccine of paragraph 39 to induce an immune response to a subject, wherein the immune response is to a pathogen from which the first polysaccharide antigen (PS1) or the second polysaccharide antigen (PS2) is derived.
43. A method to immunize a subject against a pathogen infection and/or colonization, comprising administering to the subject an immunologically effective amount of a multivalent vaccine comprising at least 2 or more species of MAPS-X immunogenic of any of paragraphs 1-34 to a subject.
44. A method to induce an immune response to a subject, comprising administering to the subject a pharmaceutical composition comprising the immune composition of any of paragraphs 1-34, or the vaccine of paragraph 39.
45. The method of paragraphs 44, wherein the immune response is an antibody or B cell response.
46. The method of paragraphs 44-45, wherein the immune response is a CD4+ T cell response, including Th1, Th2, or Th17 response, or a CD8+ T cell response, or CD4+/CD8+ T cell response.
47. The method of paragraph 41, wherein the immune response is: (a) an antibody or B cell response; and (b) a T cell response.
48. The method of any of paragraphs 44-47, wherein the immune response is to: at least the first antigenic polysaccharide, or the second antigenic polysaccharides, or both the first and second antigenic polysaccharide, or at least one polypeptide antigen.
49. The method of any of paragraphs 44-48, wherein the immune response is an antibody or B cell response to at least the first antigenic polysaccharide and/or second antigenic polysaccharide, and a CD4+ T cell response, including Th1, Th2, or Th 17 response, or a CD8+ T cell response, or CD4+/CD8+ T cell response to at least one polypeptide antigen.
50. The method of any of paragraphs 44-49, wherein the immune response is an antibody or B cell response to at least the first antigenic polysaccharide and/or the second antigenic polysaccharide, and an antibody or B cell response and a CD4+ T cell response, including Th1, Th2, or Th 17 response, or a CD8+ T cell response, or CD4+/CD8+ T cell response to at least one polypeptide antigen.
51. The immunogenic composition of any of paragraphs 1-34, wherein herein the immunogenic composition, upon administration to a subject, elicits (i) an immune response to the at least the first antigenic polysaccharide or the second antigenic polysaccharide, or both the first and second antigenic polysaccharide and (ii) an immune response to at least one of the polypeptide antigens, in the subject.
52. The immunogenic composition of paragraph 51, wherein the immune response to the at least first antigenic polysaccharide or the second antigenic polysaccharide, or both, and/or at least one of the one polypeptide antigens comprises an antibody or B cell response.
53. The immunogenic composition of paragraph 51, wherein the immune response to the at least first antigenic polysaccharide or the second antigenic polysaccharide, or both, and/or at least one of the one polypeptide antigens comprises a T cell response.
54. The immunogenic composition of paragraph 51, wherein the immune response to the at least first antigenic polysaccharide or the second antigenic polysaccharide, or both, and/or at least one of the one polypeptide antigens comprises a CD4+ T cell response, including Th1, Th2, or Th17 response, or a CD8+ T cell response, or a CD4+/CD8+ T cell response.
55. The immunogenic composition of paragraph 51, wherein the immune response to the at least first antigenic polysaccharide or the second antigenic polysaccharide, or both, and/or at least one of the one polypeptide antigens comprises: (a) an antibody or B cell response; and (b) a T cell response.
56. The immunogenic composition of any of paragraphs 1-34, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) an antibody or B cell response to at least one of polypeptide antigens, in the subject.
57. The immunogenic composition of any of paragraphs 1-34, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) an antibody or B cell response and a CD4+ T cell response, including Th1, Th2, or Th17 response, or a CD8+ T cell response, or a CD4+/CD8+ T cell response to at least one of polypeptide antigens, in the subject.
58. The immunogenic composition of any of paragraphs 1-34, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) a CD4+ T cell response, including Th1, Th2, or Th17 response, or a CD8+ T cell response, or a CD4+/CD8+ T cell response to at least one of polypeptide antigens, in the subject.
59. The immunogenic composition of any of paragraphs 1-34, wherein a biotin-binding moiety (BBM) comprises an amino acid sequence of at least 80%, or 90%, or 95% sequence identity to SEQ ID NO: 1 that has any one or more of the amino acid modifications: N80, T108, N118, S119A, N138A.
60. The immunogenic composition of any of paragraphs 1-34, wherein a biotin-binding moiety comprises an amino acid sequence of at least 80%, or 90%, or 95%, 98%, 99% or 100% sequence identity to SEQ ID NO: 8, wherein the biotin binding moiety has at least one or more of the amino acid modifications: N80, T108, N118, S119A, N138A.
61. A fusion protein comprising, or consisting of, in any order: (i) a SBD selected from any of: SBD1, SBD2, SBD3, SBD4, NanH, NanH2, NanH3, VcNanH, or a SBD comprising an amino acid sequence that at least 80%, or at least 85%, or at least 90%, or at least 95% sequence identity to any of: SEQ ID NO: 3-12, and (i) a Rhizavidin polypeptide comprising an amino acid sequence having at least 80%, or at least 85%, or at least 90%, or at least 95% sequence identity to SEQ ID NO: 1 or SEQ ID NO: 2.
62. The fusion protein of paragraph 61, comprising, or consisting essentially of: (a) an amino acid sequence having at least 80%, or at least 85%, or at least 90%, or at least 95% sequence identity to SEQ ID NO: 1 or SEQ ID NO: 2 (Rhavi), and (b) a SBD immunogenic polypeptide, selected from: (i) an amino acid sequence having at least 80%, or at least 85%, or at least 90%, or at least 95% sequence identity to SEQ ID NO: 3 (SBD1), (ii) an amino acid sequence having at least 80%, or at least 85%, or at least 90%, or at least 95% sequence identity to SEQ ID NO: 4 (SBD2), (iii) an amino acid sequence having at least 80%, or at least 85%, or at least 90%, or at least 95% sequence identity to SEQ ID NO: 5 (SBD3), (iv) an amino acid sequence having at least 80%, or at least 85%, or at least 90%, or at least 95% sequence identity to SEQ ID NO: 6 (SBD4), (v) an amino acid sequence having at least 80%, or at least 85%, or at least 90%, or at least 95% sequence identity to SEQ ID NO: 7 (NanH), (vi) an amino acid sequence having at least 80%, or at least 85%, or at least 90%, or at least 95% sequence identity to SEQ ID NO: 8 (NanH2), or (vii) an amino acid sequence having at least 80%, or at least 85%, or at least 90%, or at least 95% sequence identity to SEQ ID NO: 9 (NanH3).
63. The fusion protein of any of paragraphs 60-62, is selected from any of the fusion proteins comprising an amino acid sequence having at least 80%, or at least 85%, or at least 90%, or at least 95% sequence identity to any of: SEQ ID NO: 9-20.
64. The fusion protein of any of paragraphs 60-62, further comprising at least one linker sequence or at least one spacer sequence located between any of: the BBM and the SBD.
65. The fusion protein of any of paragraphs 60-64, wherein the linker sequence or spacer sequence is selected from any of: GGGGSSS, TDPNSSS, SSS, AAA.
66. The fusion protein of any of paragraphs 60-65, wherein the fusion protein comprises the polypeptide of SEQ ID NO: 13-20, or amino acid sequence having at least 80%, or at least 85%, or at least 90%, or at least 95% sequence identity to any of SEQ ID NOs: 13-20.
67. The fusion protein of any of paragraphs 60-66, wherein the fusion protein consists of the polypeptide of SEQ ID NO: 13-20, or amino acid sequence having at least 80%, or at least 85%, or at least 90%, or at least 95% sequence identity to any of SEQ ID NOs: 13-20.
68. An expression vector comprising a nucleic acid encoding a fusion protein of any of paragraphs 61-67, wherein the expression vector comprises a promoter operatively linked to a nucleic acid sequence encoding the fusion protein, wherein the nucleic acid sequence comprises, in any order: (i) a nucleic acid sequence encoding a Rhizavidin protein comprising amino acids of SEQ ID NO: 1 or SEQ ID NO: 2 or a protein having at least 80% sequence identity to SEQ ID NO: 1 or SEQ ID NO: 2, and (ii) a nucleic acid sequence encoding a at least one sialic acid binding domain (SBD) selected from any of: SBD1, SBD2, SBD3, SBD4, NanH, NanH2, NanH3, or VcNanH, or a SBD having an amino acid sequence that has at least 80%, or at least 85%, or at least 90%, or at least 95% sequence identity to any of: SEQ ID NO: 2-12.
69. The expression vector of paragraph 68, wherein the nucleic encoding a Rhizavidin protein comprises a nucleic acid sequence having at least 80%, or at least 85%, or at least 90%, or at least 95% sequence identity to SEQ ID NO: 78 or a codon optimized variant thereof.
70. The expression vector of paragraph 68, wherein the nucleic encoding a SBD protein comprises a nucleic acid sequence having at least 80%, or at least 85%, or at least 90%, or at least 95% sequence identity to any of: SEQ ID NO: 80-84, or codon optimized variants thereof.
71. A cell comprising the expression vector of any of paragraphs 68-70.
72. A method of manufacturing a fusion protein of any of paragraphs 61-67, using the expression vector of any of paragraphs 68-70.

### XII. Certain Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastrical, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: In its broadest sense, the term “amino acid”, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Non-standard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kDa tetrameric agents comprised of two identical heavy chain polypeptides (about 50 kDa each) and two identical light chain polypeptides (about 25 kDa each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Antigen: The term “antigen”, as used herein, refers to (i) an agent that induces an immune response; and/or (ii) an agent that binds to a T cell receptor (e.g., when presented by an MHC molecule) or to an antibody. In some embodiments, an antigen induces a humoral response (e.g., including production of antigen-specific antibodies); in some embodiments, an antigen induces a cellular response (e.g., involving T cells whose receptors specifically interact with the antigen). In some embodiments, an antigen induces a humoral response and a cellular response. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)), etc. In some embodiments, an antigen is or comprises a polypeptide. In some embodiments, an antigen is or comprises a polysaccharide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen. In some embodiments, an antigen is a polypeptide or a polysaccharide that, upon administration to a subject, induces a specific and/or clinically relevant immune response to such polypeptide or polysaccharide. In some embodiments, an antigen is selected to induce a specific and/or clinically relevant immune response to such polypeptide or polysaccharide.

Associated with: Two entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another. In some embodiments, two or more entities that are physically associated with one another are not covalently linked to one another but are non-covalently associated, for example by means of affinity interactions, electrostatic interactions, hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Carrier protein: As used herein, the term “carrier protein” refers to a protein or peptide that is coupled, complexed, or otherwise associated with a hapten (e.g., a small peptide or lipid) or less immunogenic antigen (e.g., a polysaccharide) and that induces or improves an immune response to such a coupled, or complexed, or otherwise associated hapten (e.g., a small peptide or lipid) or less immunogenic antigen (e.g., a polysaccharide). In some embodiments, such an immune response is or comprises a response to a hapten or less immunogenic antigen that is coupled, complexed, or otherwise associated with such a carrier protein. In some embodiments, such an immune response is or comprises a response to both a carrier protein and a hapten or less immunogenic antigen that is coupled, complexed, or otherwise associated with such a carrier protein. In some embodiments, no significant immune response to a carrier protein itself occurs. In some embodiments, immune response to a carrier protein may be detected; in some such embodiments, immune response to such a carrier protein is strong. In some embodiments, a carrier protein is coupled, complexed, or otherwise associated with one or more other molecules.

Colonization: As used herein, the term “colonization” generally refers to the ability of a microbe to grow at a target site or surface. For example, the term “colonization” refers to the ability of a microbe (e.g., a bacterium) to grow at an anatomical site (e.g., a mucosal membrane, gastrointestinal tract, injury site, organ, etc.) of a host.

Combination therapy: As used herein, the term “combination therapy” refers to those situations in which a subject is exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Derivative: As used herein, the term “derivative”, or grammatical equivalents thereof, refers to a structural analogue of a reference substance. That is, a “derivative” is a substance that shows significant structural similarity with the reference substance, for example sharing a core or consensus structure, but also differs in certain discrete ways. Such a substance would be said to be “derived from” said reference substance. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, R-sheet character, coiled-coil character, random coil character, etc.), and/or by a particular functional feature (e.g., binding activity, enzymatic activity, folding activity, signaling activity, etc.).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment includes a discrete portion of the whole which discrete portion shares one or more functional characteristics found in the whole. In some embodiments, a fragment consists of such a discrete portion. In some embodiments, a fragment consists of or comprises a characteristic structural element or moiety found in the whole. In some embodiments, a fragment of a polymer, e.g., a polypeptide or polysaccharide, comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, or 99% of the monomeric units (e.g., residues) found in the whole polymer. The whole material or entity may in some embodiments be referred to as the “parent” of the whole.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98% or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In some embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, at least 99.5%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, inhibit or reduce: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Immunologically effective amount or immunologically effective dose: As used herein, “immunologically effective amount” or “immunologically effective dose” refers to an amount of an antigenic or immunogenic substance, e.g., an antigen, immunogen, immunogenic complex, immunogenic composition, vaccine, or pharmaceutical composition, which when administered to a subject, either in a single dose or as part of a series of doses, that is sufficient to enhance a subject's own immune response against a subsequent exposure to a pathogen. In some embodiments, the pathogen is S. agalactiae (Group B strep). In some embodiments, the immune response is against one or more different serotypes of S. agalactiae (Group B strep). In some embodiments, the immune response is against two or more different serotypes of S. agalactiae (Group B strep). In some embodiments, the immune response is against four or more different serotypes of S. agalactiae (Group B strep). In some embodiments, the immune response is against five or more different serotypes of S. agalactiae i some embodiments, the immune response is against six or more different serotypes of S. agalactiae (Group B strep). In some embodiments, the immune response is against seven or more different serotypes of S. agalactiae (Group B strep). In some embodiments, the immune response is against eight or more different serotypes of S. agalactiae (Group B strep). An immunologically effective amount may vary based on the subject to be treated, the species of the subject, the degree of immune response desired to induce, etc. In some embodiments, an immunologically effective amount is sufficient for treatment or protection of a subject having or at risk of having disease. In some embodiments, an immunologically effective amount refers to a non-toxic but sufficient amount that can be an amount to treat, attenuate, or prevent infection and/or disease (e.g., bacterial infection, S. agalactiae infection, bacterial colonization, S. agalactiae colonization, complications associated with bacterial infection, complications associated with S. agalactiae infection, etc.) in any subject. In some embodiments, an immunologically effective amount is sufficient to induce an immunoprotective response upon administration to a subject.

Immunoprotective response or protective response: As used herein, “immunoprotective response” or “protective response” refers to an immune response that mediates antigen or immunogen-induced immunological memory. In some embodiments, an immunoprotective response is induced by the administration of a substance, e.g., an antigen, immunogen, immunogenic complex, immunogenic composition, vaccine, or pharmaceutical composition to a subject. In some embodiments, immunoprotection involves one or more of active immune surveillance, a more rapid and effective response upon immune activation as compared to a response observed in a naïve subject, efficient clearance of the activating agent or pathogen, followed by rapid resolution of inflammation. In some embodiments, an immunoprotective response is an adaptive immune response. In some embodiments, an immunoprotective response is sufficient to protect an immunized subject from productive infection by a particular pathogen or pathogens to which a vaccine is directed (e.g., S. agalactiae (Group B strep) infection).

Immunization: As used herein, “immunization”, or grammatical equivalents thereof, refers to a process of inducing an immune response to an infectious organism or agent in a subject (“active immunization”), or alternatively, providing immune system components against an infectious organism or agent to a subject (“passive immunization”). In some embodiments, immunization involves the administration of one or more antigens, immunogens, immunogenic complexes, vaccines, immune molecules such as antibodies, immune sera, immune cells such as T cells or B cells, or pharmaceutical compositions to a subject. In some embodiments, immunization is performed by administering an immunologically effective amount of a substance, e.g., an antigen, immunogen, immunogenic complex, immunogenic composition, vaccine, immune molecule such as an antibody, immune serum, immune cell such as a T cell or B cell, or pharmaceutical composition to a subject. In some embodiments, immunization results in an immunoprotective response in the subject. In some embodiments, active immunization is performed by administering to a subject an antigenic or immunogenic substance, e.g., an antigen, immunogen, immunogenic complex, vaccine, or pharmaceutical composition. In some embodiments, passive immunization is performed by administering to a subject an immune system component, e.g., an immune molecule such as an antibody, immune serum, or immune cell such as a T cell or B cell.

Isolated: As used herein, the term “isolated”, or grammatical equivalents thereof, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polysaccharide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide or polysaccharide that is chemically synthesized or is synthesized in a cellular system different from that which produces it in nature is considered to be an “isolated” polypeptide or polysaccharide. Alternatively or additionally, in some embodiments, a polypeptide or polysaccharide that has been subjected to one or more purification techniques may be considered to be an “isolated” polypeptide or polysaccharide to the extent that it has been separated from other components a) with which it is associated in nature; and/or b) with which it was associated when initially produced.

Linker: As used herein, the term “linker” is used to refer to an entity that connects two or more elements to form a multi-element agent. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

Pathogen: As used herein the term “pathogens” may include, for example, viral, bacterial, protozoan and/or fungal pathogens, some of which may possess an ability to interact and/or associate with and/or bind to, a host cell sialic acid containing receptor. Thus the MAPS-X immunogenic complex as disclosed herein is useful on an application in the treatment and/or prevention of respiratory diseases/infections caused or contributed to by viral, bacterial, protozoan and/or fungal pathogens, some of which may exploit the presence of sialic acid containing receptors on the surface of epithelial cells lining the surface of the upper and lower respiratory tracts.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Plurality: As used herein, the term “plurality” includes at least 2 or more, including, e.g., at least 3, at least 4, at least 5, at least 6, at least 7, at least 8, at least 9, at least 10, or more.

Polysaccharide: The term “polysaccharide” as used herein refers to a polymer macromolecule that is a polymeric carbohydrate molecule composed of long chains of monosaccharide units bound together by glycosidic, phosphodiester, or other linkages, and on hydrolysis give the constituent monosaccharides or oligosaccharides. Polysaccharides range in structure from linear to highly branched. Examples include storage polysaccharides such as starch and glycogen, structural polysaccharides such as cellulose and chitin and microbial polysaccharides, and antigenic polysaccharides found in microorganisms including, but not limited to, capsular polysaccharides (CPS), O polysaccharides (OPS), core O polysaccharides (COPS), and lipopolysaccharides (LPS). Polysaccharides comprise regions of polysaccharide antigens, as this term is disclosed herein.

Polysaccharide Antigen: The term “polysaccharide antigen” as used herein refers to a region or portion of a polysaccharide macromolecule that comprises a plurality of biotin molecules and/or sialic acid molecule. That is a “polysaccharide antigen” is a region in the polysaccharide macromolecule to which a SBD and/or BBM non-covalently associates with.

Polypeptide: The term “polypeptide”, as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Prevention: The term “prevent” or “prevention”, as used herein in connection with a disease, disorder, and/or medical condition, refers to reducing the risk of developing the disease, disorder and/or condition, and/or a delay of onset, and/or reduction in frequency and/or severity of one or more characteristics or symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete when onset of a disease, disorder or condition has been delayed for a predefined period of time.

Protein: As used herein, the term “protein” encompasses a polypeptide. Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant: As used herein, the term “recombinant” is intended to refer to polypeptides that are designed, engineered, prepared, expressed, created, manufactured, and/or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, subject, population, sample, sequence or value of interest is compared with a reference or control agent, animal, subject, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a “response” to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Subject or tumor response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of biomarkers in a sample obtained from a subject, cytology, and/or histology. The exact response criteria can be selected in any appropriate manner, provided that when comparing groups of subjects and/or tumors, the groups to be compared are assessed based on the same or comparable criteria for determining response rate. One of ordinary skill in the art will be able to select appropriate criteria.

Risk: As will be understood from context, “risk” of a disease, disorder, and/or condition refers to a likelihood that a particular subject will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments, risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from subjects comparable to a particular subject. In some embodiments, relative risk is 0, 1, 2, 3, 4, 5, 6, 7, 8, 9, 10, or more.

Serotype: As used herein, the term “serotype”, also referred to as a serovar, refers to a distinct variation within a species of bacteria or virus or among immune cells of different subjects. These microorganisms, viruses, or cells are classified together based on their cell surface antigens, allowing the epidemiologic classification of organisms to the sub-species level. A group of serovars with common antigens may be referred to as a serogroup or sometimes serocomplex.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered. Thus, the “subject” (whether animal or human), may be symptomatic and/or (suspected of) suffering from an infection or disease by a pathogen as disclosed herein, for example a respiratory infection or disease. Alternatively, the subject may be asymptomatic and/or predisposed/susceptible to an infection or disease by the pathogen, for example, a respiratory infection or disease. In all cases, the infection or disease may have a microbial (for example bacterial, viral, protozoan and/or fungal aetiology.

Susceptible to: A subject who is “susceptible to” a disease, disorder, or condition is at risk for developing the disease, disorder, or condition. In some embodiments, a subject who is susceptible to a disease, disorder, or condition does not display any symptoms of the disease, disorder, or condition. In some embodiments, a subject who is susceptible to a disease, disorder, or condition has not been diagnosed with the disease, disorder, and/or condition. In some embodiments, a subject who is susceptible to a disease, disorder, or condition is a subject who has been exposed to conditions associated with development of the disease, disorder, or condition. In some embodiments, a risk of developing a disease, disorder, and/or condition is a population-based risk (e.g., family members of subjects suffering from the disease, disorder, or condition).

Symptoms are reduced: As used herein, “symptoms are reduced” when one or more symptoms of a particular disease, disorder or condition is reduced in magnitude (e.g., intensity, severity, etc.) and/or frequency, e.g., to a statistically and/or clinically significant or relevant level. For purposes of clarity, a delay in the onset of a particular symptom is considered one form of reducing the frequency of that symptom.

Treatment: As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vaccination: As used herein, the term “vaccination” refers to the administration of a composition intended to generate an immune response, for example to a disease-causing agent. For the purposes of the present invention, vaccination can be administered before, during, and/or after exposure to a disease-causing agent, and in some embodiments, before, during, and/or shortly after exposure to the agent. In some embodiments, vaccination includes multiple administrations, appropriately spaced in time, of a vaccinating composition. In some embodiments, vaccination initiates immunization. The terms “vaccine” or “vaccine composition”, which are used interchangeably, refer to pharmaceutical compositions comprising at least one immunogenic composition that induces an immune response in an animal.

### EXAMPLES

### Example 1

Use of a Bifunctional SBD-BBM Fusion Protein, where SBD is Incorporated in the Carrier Protein Significantly Enhances Antibody to GBS Polysaccharide.

As shown in FIG. 3A, a MAPS-X immunogenic composition comprising a bifunctional SBD-BBM fusion protein (see FIG. 1B) was compared to a MAPS immunogenic composition comprising a BBM fusion protein that does not comprise SBD. The MAPS-X complex was significantly larger as compared to other MAPS immunogenic complexes, including a MAPS complex comprising a Rhizavidin as a BBM, or a MAPS comprising distinct (non-fused) Rhizavidin and SBD proteins.

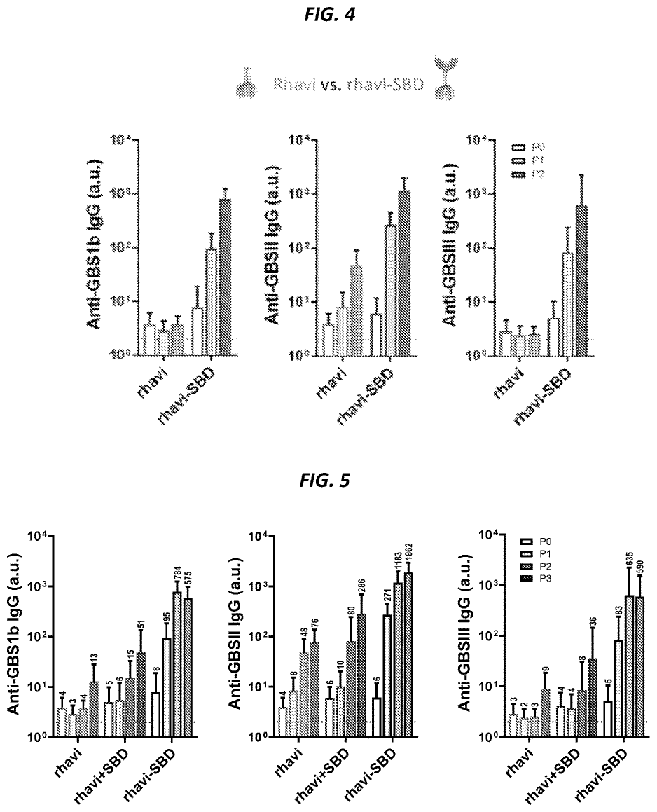

Rabbits were immunized with a trivalent (3V) GBS MAPS vaccine comprising GBS polysaccharides Ib, II and III, and comprising either (i) a Rhavi-SBD fusion protein or (ii) a rhizavidin protein (where Rhizavidin is not fused to another protein, including an antigen or SBD). Mice were immunized two times with three weeks intervals. Rabbits were bleed at day 0 (P0), day 21 (p1) or day 42 (p2). The antibody titers against three GBS polysaccharides (Ib, II and III) were measured using ELISA. As shown in FIG. 4, rabbits immunized with MAPS containing Rhavi-SBD made higher antibodies than those immunized with MAPS comprising a rhizavidin protein (non-fused Rhizavidin)

MAPS immunogenic complexes comprising either (i) a Rhavi-SBD fusion protein (MAPS-X) or (ii) a rhizavidin protein (where is not fused to another protein), or (iii) Rhavi and SBD as separate polypeptides was used to confirm the optimal dose to induce anti-PS antibody. As shown in FIG. 5, the MAPS-X vaccine comprising the bifunctional SBD-BBM fusion protein induced the greatest and a robust antibody responses to GBS polysaccharides I, II and III after one injection for all the doses tested.

### Example 2

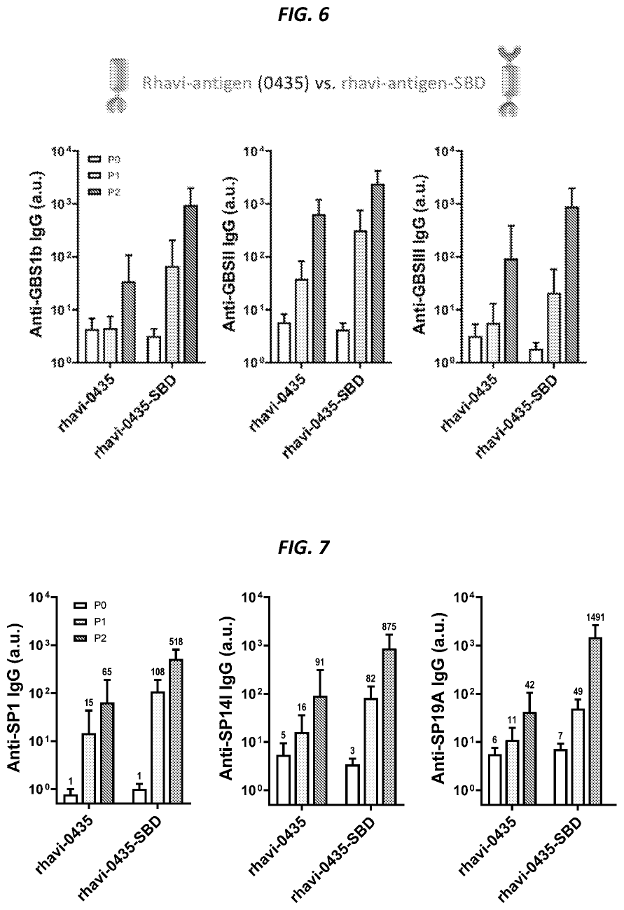

Next, the addition of a polypeptide antigen in the bifunctional SBD-BBM fusion protein was assessed. Rabbits were immunized with a tri-valent GBS MAPS vaccine (3V GBS MAPS) comprising either (i) Rhavi-0435 or (ii) Rhavi-0435-SBD fusion proteins two times with three weeks intervals. Rabbits were bleed at day 0 (P0), day 21 (p1) or day 42 (p2). The antibody titers against three GBS polysaccharides (Ib, II and III) were measured using ELISA. As shown in FIG. 6 rabbits immunized with a GBS MAPS containing Rhavi-0435-SBD made higher antibodies than those immunized with MAPS containing Rhavi-0435. This demonstrates that the bifunctional SBD-BBM fusion protein can comprise one or more polypeptide antigens (e.g., a SBD[Ag]-BBM fusion protein), and can enhance an antibody response to polysaccharides comprising sialic acid on their surface (e.g., such as GBS), as compared to MAPS immunogenic complexes that comprise a Rhavi fusion protein (e.g., a BBM-Ag fusion protein) that is not fused to SBD.

### Example 3

GBS polysaccharides naturally comprise sialic acid on their surface. In order to assess if a bifunctional SBD-BBM fusion protein in a MAPS-X complex can enhance an immune response and/or production of antibodies to polysaccharides that do not have sialic acid on the surface, the inventors assessed a 3V MAPS complex comprising a pneumococcal polysaccharides from serotypes 1, 14, and 19A.

Similar to Example 2, rabbits were immunized this time with a tri-valent pneumococcal MAPS vaccine (3V MAPS-pneumoniae) containing either (i) Rhavi-0435 or (ii) Rhavi-0435-SBD fusion proteins two times with three weeks intervals. Rabbits were bleed at day 0 (P0), day 21 (p1) or day 42 (p2). The antibody titers against three pneumococcal polysaccharides (1, 14, 19A) were measured using ELISA. Surprisingly, as shown in FIG. 7 rabbits immunized with a MAPS-pneumoniae containing Rhavi-0435-SBD made higher antibodies than those immunized with MAPS containing Rhavi-0435. This demonstrates that the bifunctional SBD-BBM fusion protein can surprisingly enhance an antibody response to polysaccharides that do not comprise sialic acid on their surface (e.g., such as pneumococcal polysaccharides), as compared to MAPS immunogenic complexes that comprise a Rhavi fusion protein (e.g., a BBM-Ag fusion protein) that is not fused to SBD.

### Example 2

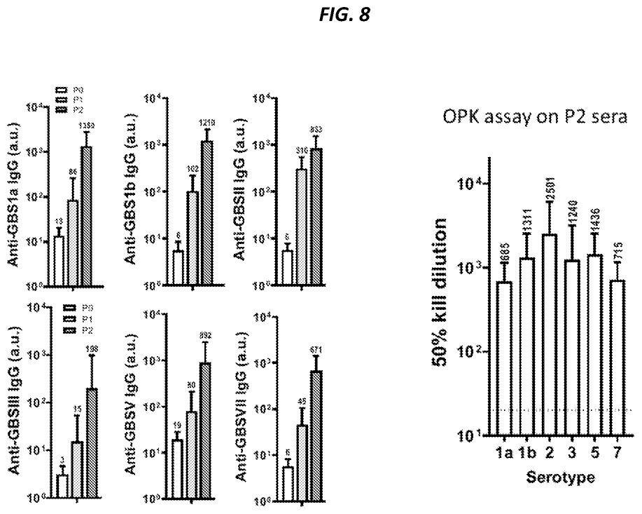

A 6-valent GBS MAPS-X vaccine induced functional antibody response to all the GBS polysaccharides. Rabbits were immunized with a 6 valent GBS MAPS-X composition containing serotypes Ia, Ib, II, III, V and VII two times with three weeks intervals. Antibody titer against each serotype was measured by ELISA (FIG. 8). The post-2 immune sera were used in an OPK assay with GBS clinical strains. MAPS-GBS induced high killing titer for all six serotypes (FIG. 8). OPK titer of pre-immune sera (P0) was below the lower limit of detection (20) for all serotypes.

### Example 3

### Surface Exposure and Function of GBS Antigens

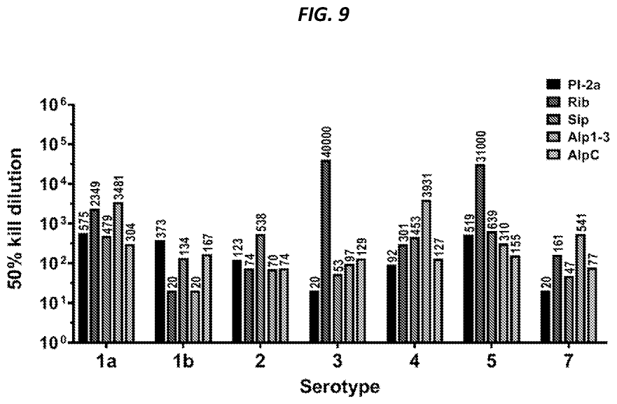

The inventors assessed different GBS polypeptide antigens for use as an exemplary SBD[Ag]-BBM fusion protein. Antisera against PI-2a, Rib, Sip or AlpC were used in a flow cytometry analysis to determine the exposure of each protein on the surface of GBS with six different serotypes. As shown in FIG. 9, Rib sera had the highest surface binding to type III and V, while AlpC sera bound to type Ib, II and VII. These sera with the addition of anti-sera against Alp1-3 fusion protein, were used in OPK assay against GBS clinical strains (7 serotypes). Rib sera had high OPK titer against type III and V, consistent with the flow cytometry analysis (data not shown).

### Example 4

### Effect of Dose and Immunization Schedule on PS Immunogenicity.

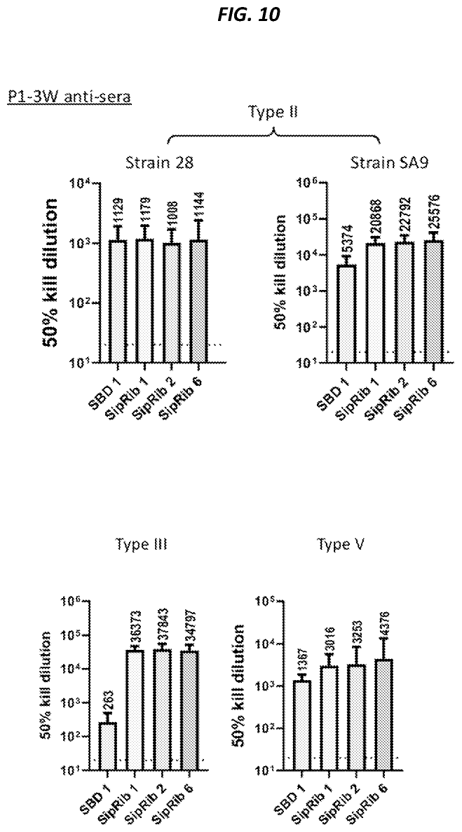

A MAPS-X immunogenic complexes comprising Rhavi-SBD and Rhavi-Sip-Rib-SBD as exemplary SBD[Ag]-BBM fusion proteins, were used to immunize rabbits. Anti-PS and anti-Rib, anti-Sip ELISA titers were analyzed at 3 weeks or 6 weeks after one immunization. The anti-PS titer was higher at 6 weeks post one immunization, and 1-2 ug per dose is optimal for Rhavi-Sip-Rib-SBD MAPS (data not shown). In contrast, anti-protein antibody stayed flat at 3 weeks or 6 weeks post immunization (data not shown). Assessment of the killing titer of antisera from the immunization of Rhavi-Sip-Rib-SBD MAPS-X against serotypes II (strains 28 and SA9), III, and V GBS stains was also performed. As shown in FIG. 10, Rhavi-Sip-Rib-SBD MAPS-X induced higher killing activity against all three serotypes.

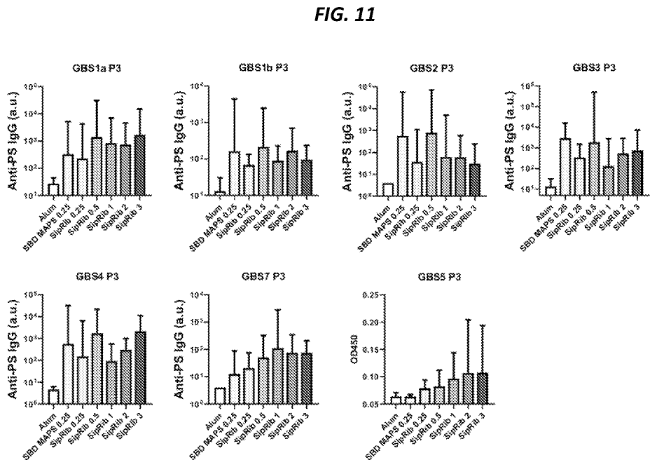

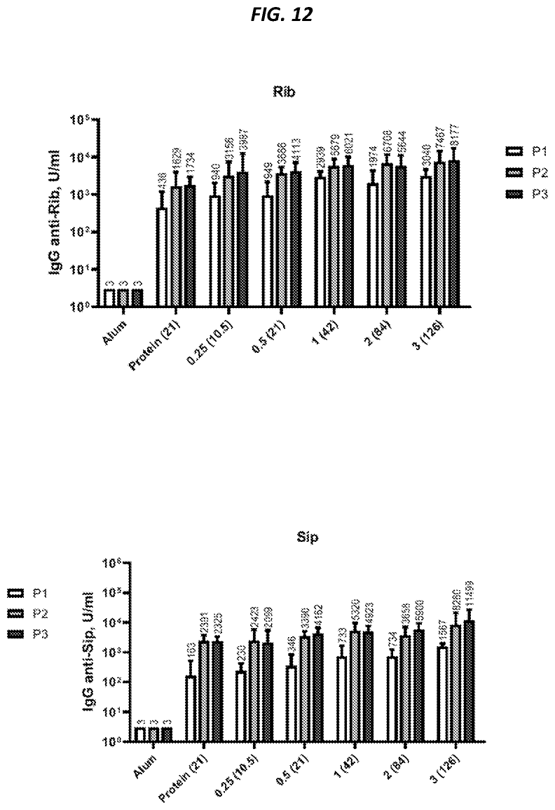

A seven-valent GBS MAPS-X composition comprising Rhavi-Sip-Rib-SBD was used to confirm the optimal dose to induce anti-PS antibody. As shown in FIG. 11, the seven valent MAPS-X vaccine induced robust antibody responses to all GBS polysaccharides after one injection for all the doses tested. The antibody response to Rib and Sip proteins were also measured by ELISA. As shown in FIG. 12, antibody response was peaked after two immunizations.

Accordingly, the inventors demonstrate that a MAPS-X complex comprising a bifunctional fusion protein comprising SBD and Rhizavidin fusion protein produces a greater immune response to polysaccharides in the MAPS complex as compared to a MAPS complex comprising a Rhavi fusion protein that does not comprise a SBD. Moreover, the inventors also demonstrate that a MAPS-X complex comprising a bifunctional fusion protein comprising SBD and Rhizavidin fusion protein as disclosed herein increases the immune response and production of antibodies to polysaccharides that comprise sialic acid on their surface (e.g., such as but not limited to GBS) and also, surprisingly, pneumococcal polysaccharides, as compared to a MAPS complex comprising a Rhavi fusion protein that does not comprise a SBD.

### REFERENCES

All the references cited herein and throughout the application are incorporated by reference in their entirety.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

1. An immune composition comprising one or more species of MAPS-X immunogenic complexes,
wherein each species of the MAPS-X immunogenic complex comprises:
a. at least a first biotinylated antigenic polysaccharide (PS1) from a pathogen,
b. at least a second biotinylated antigenic polysaccharide (PS2) from a pathogen,
c. at least a first fusion protein comprising, or consisting of: a biotin-biotin moiety (BBM) and a sialic acid binding moiety (SBD) (SBD-BBM fusion protein),
wherein the BBM of the at least first fusion protein non-covalently associates with at least one biotin molecule on the first biotinylated antigenic polysaccharide (PS1), and the SBD non-covalently associates with the second antigenic polysaccharide, or
wherein a SBD of the at least first fusion protein non-covalently associates with the first biotinylated antigenic polysaccharide and the BBM non-covalently associates with at least biotin molecule on the second biotinylated antigenic polysaccharide,
wherein the first biotinylated antigenic polysaccharide and the second biotinylated antigenic polysaccharide are indirectly joined by their non-covalent association with the first fusion protein to form a MAPS-X immunogenic complex.

2. The immune composition of claim 1, wherein the PS1 and PS2 are located on the same polysaccharide macromolecule.

3. The immune composition of claim 1, wherein the PS1 and PS2 are located on distinct polysaccharide macromolecules.

4. The immune composition of claim 3, wherein one polysaccharide macromolecule is from a pathogen, and the other macromolecule is from a distinct pathogen or serotype.

5. The immune composition of any of claims 1-4, wherein the first or second biotinylated antigenic polysaccharide, or both, comprise a sialic acid molecule.

6. The immune composition of any of claims 1-5, wherein the first or second biotinylated antigenic polysaccharide, or both, do not comprise a sialic acid molecule.

7. The immune composition of any of claims 1-6, wherein the SBD of the first fusion protein associates with sialic acid on the first or second antigenic polysaccharide, and/or a negatively charged first or second antigenic polysaccharide.

8. The immune composition of any of claim 1-8, wherein each MAPS-X immunogenic complex further comprises at least a second fusion protein,
wherein if the BBM of the at least first fusion protein non-covalently associates with at least one biotin molecule on the first biotinylated antigenic polysaccharide (PS1), then the BBM of the at least second fusion protein non-covalently associates with at least one biotin molecule on the second biotinylated antigenic polysaccharide (PS2), and the SBD non-covalently associates with the first antigenic polysaccharide (PS1), or
wherein if the BBM of the at least first fusion protein non-covalently associates with at least one biotin molecule on the second biotinylated antigenic polysaccharide (PS2), then the BBM of the at least second fusion protein non-covalently associates with at least one biotin molecule on the first biotinylated antigenic polysaccharide (PS1), and the SBD non-covalently associates with the second antigenic polysaccharide (PS2).

9. The immune composition of any of claims 1-8, wherein the MAPS-X immunogenic complex further comprises (i) a plurality of first biotinylated antigenic polysaccharides, and (ii) a plurality of second antigenic polysaccharides, and (ii) a plurality of first fusion proteins and a plurality of second fusion proteins, wherein each first biotinylated antigenic polysaccharide can be indirectly joined to more than one second antigenic polysaccharides by more than one first or second fusion protein, and vice versa, each second biotinylated antigenic polysaccharide can be indirectly joined to more than one first antigenic polysaccharides by more than one first or second fusion protein.

10. The immune composition of any of claims 1--9, wherein the first antigenic polysaccharide and the second antigenic polysaccharide in a species MAPS-X immunogenic complex are from the same pathogenic organism.

11. The immune composition of any of claims 1-10, wherein the first antigenic polysaccharide and the second antigenic polysaccharide in a species of MAPS-X immunogenic complex are from a different pathogenic organism.

12. The immune composition of any of claims 1-11, wherein the first antigenic polysaccharide and the second antigenic polysaccharide in of MAPS-X immunogenic complex are from the same subtype of pathogenic organism, or from a different subtype of a pathogenic organism.

13. The immune composition of any of claims 1-12, comprising:
a. two or more species of MAPS-X immunogenic complexes, each species of MAPS-X immunogenic complex comprises a first antigenic polysaccharide and second antigenic polysaccharide that is from a distinct pathogenic organism, and/or
b. two or more species of MAPS-X immunogenic complexes, each species of MAPS-X immunogenic complex comprises a first antigenic polysaccharide and second antigenic polysaccharide that is from a distinct subtype or serotype of the same pathogenic organism.

14. The immune composition of any of claims 1-13, wherein the immune composition comprises at least 2, or at least 3, or at least 4, or at least 5 or more than 5 distinct species of MAPS-X immunogenic complexes.

15. The immune composition of any of claims 1-14, wherein the immune composition comprises at least 6-10, 11-15, 16-20, 21-25, 26-30, or 31-35 or more than 35 distinct species of MAPS-X immunogenic complexes.

16. The immune composition of any of claims 1-15, wherein the first or second fusion protein further comprise at least a one polypeptide antigen or an antigenic fragment thereof, where the biotin binding moiety (BBM), sialic acid binding moiety (SBD), and at least one polypeptide antigen or an antigenic fragment thereof can be in any order.

17. The immune composition of any of claims 1-16, wherein each species of MAPS-X immunogenic complex further comprises:
a. a third fusion protein comprising, in any order, a biotin binding moiety (BBM), and at least one polypeptide antigen or an antigenic fragment thereof, and/or
b. a fourth fusion protein comprising, in any order, a sialic acid binding moiety (SBD), and at least one polypeptide antigen or an antigenic fragment thereof.

18. The immune composition of any of claims 1-17, wherein the BBM is Rhizavidin and comprises an amino acid sequence having at least 80%, or at least 85%, or at least 90%, or at least 95% sequence identity to SEQ ID NO: 1 or SEQ ID NO: 2.

19. The immune composition of any of claims 1-18, wherein SBD is selected from any of: SBD1, SBD2, SBD3, SBD4, NanH, NanH2, NanH3, VcNanH, or a SBD comprising an amino acid sequence that at least 80%, or at least 85%, or at least 90%, or at least 95% sequence identity to any of: SEQ ID NO: 3-12.

20. The immune composition of any of claims 1-19, wherein the first or second fusion protein comprises,
a. an amino acid sequence having at least 80%, or at least 85%, or at least 90%, or at least 95% sequence identity to SEQ ID NO: 1 or SEQ ID NO: 2 (Rhavi), and
b. a SBD polypeptide, selected from:
i. an amino acid sequence having at least 80%, or at least 85%, or at least 90%, or at least 95% sequence identity to SEQ ID NO: 3 (SBD1),
ii. an amino acid sequence having at least 80%, or at least 85%, or at least 90%, or at least 95% sequence identity to SEQ ID NO: 4 (SBD2),
iii. an amino acid sequence having at least 80%, or at least 85%, or at least 90%, or at least 95% sequence identity to SEQ ID NO: 5 (SBD3),
iv. an amino acid sequence having at least 80%, or at least 85%, or at least 90%, or at least 95% sequence identity to SEQ ID NO: 6 (SBD4),
v. an amino acid sequence having at least 80%, or at least 85%, or at least 90%, or at least 95% sequence identity to SEQ ID NO: 7 (NanH),
vi. an amino acid sequence having at least 80%, or at least 85%, or at least 90%, or at least 95% sequence identity to SEQ ID NO: 8 (NanH2), or
vii. an amino acid sequence having at least 80%, or at least 85%, or at least 90%, or at least 95% sequence identity to SEQ ID NO: 9 (NanH3).

21. The immune composition of any of claims 1-20, wherein the first or second fusion protein is selected from a fusion protein comprising an amino acid sequence having at least 80%, or at least 85%, or at least 90%, or at least 95% sequence identity to any of: SEQ ID NO: 9-20.

22. The immunogenic composition of any of claims 1-22, wherein the first or second fusion protein comprises at least one linker sequence or at least one spacer sequence located between any of: the BBM and the SBD; the BBM and at the first polypeptide antigen or an antigenic fragment thereof, the first polypeptide antigen or an antigenic fragment thereof and the second polypeptide antigen or an antigenic fragment thereof, or a polypeptide antigen or an antigenic fragment thereof and a SBD.

23. The immunogenic composition of any of claims 1-22, wherein the linker sequence or spacer sequence is selected from any of: GGGGSSS, TDPNSSS, SSS, AAA.

24. The immunogenic composition of any of claims 1-23, wherein the polypeptide antigen or fragment thereof is from the same pathogen as the first or second, or both polysaccharides.

25. The immunogenic composition of any of claims 1-24, wherein the polypeptide antigen or fragment thereof is from a different pathogen to the pathogen of the first or second polysaccharides.

26. The immunogenic composition of any of claims 1-25, wherein the first or second polysaccharide, or both, is selected from the group consisting of: polysaccharides, oligosaccharides, or lipopolysaccharides from Gram-positive bacteria; polysaccharides, oligosaccharides, or lipopolysaccharides from Gram-negative bacteria; other bacterial capsular or cell wall polysaccharides; fungal polysaccharides; viral polysaccharides; and polysaccharides derived from cancer or tumor cells.

27. The immunogenic composition of any of claims 1-26, wherein the first or second antigenic polysaccharide, or both, are selected from the group consisting of: Salmonella typhi Vi capsular polysaccharides; Salmonella polysaccharides; Shigella polysaccharide, pneumococcal polysaccharides; Haemophili polysaccharides; Meningococcal polysaccharides; Staphylococcus aureus polysaccharides; Bacillus anthracis polysaccharides; Streptococcus polysaccharides; Pseudomonas polysaccharides; Cryptococcus polysaccharides; and viral glycoproteins.

28. The immunogenic composition of any of claims 1-27, wherein the first or second antigenic polysaccharide, or both, are pneumococcal capsular polysaccharides.

29. The immunogenic composition of claim 28, comprising at least 15 or more species of MAPS-X immunogenic composition, wherein each species of MAPS-X immunogenic complex comprises a first antigenic polysaccharide and second antigenic polysaccharide that is from a distinct subtype of Streptococcus pneumoniae.

30. The immunogenic composition of claim 25, wherein the first and second immunogenic polysaccharide for each species of MAPS-X immunogenic complex are selected from Streptococcus pneumoniae serotypes: 1, 2, 3, 4, 5, 6A, 6B, 6C, 6D, 6E, 6F, 6G, 6H, 7A, 7B, 7C, 7F, 8, 9A, 9L, 9N, 9V, 10A, 10B, 10C, 11F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F, and 48.

31. The immune composition of any of claims 1-30, wherein the first or second antigenic polysaccharide, or both, is a capsular polysaccharide (CP) from a Group B Streptococcus (GBS) or Streptococcus agalactiae.

32. The immunogenic composition of claim 31, wherein the first or second immunogenic polysaccharide, or both for each species of MAPS-X immunogenic complex are selected from Streptococcus agalactiae serotypes Ia, Ib, II, III, IV, V, VI, VII, VIII, and IX,

33. The immune composition of any of claims 1-32, wherein the first or second antigenic polysaccharide, or both, is not from a Group B Streptococcus (GBS) or Streptococcus agalactiae.

34. The immune composition of any of claims 1-33, wherein the first biotinylated polysaccharide, or second biotinylated polysaccharide, or both, has a sialic acid level of greater than about 20% or about 30%, or about 40%, or about 50% or about 60%.

35. A pharmaceutical composition comprising the immunogenic composition of any of claims 1-34, and a pharmaceutically acceptable carrier.

36. The pharmaceutical composition of claim 35, further comprising one or more adjuvants.

37. The pharmaceutical composition of claim 36, wherein the one or more adjuvants is or comprises a co-stimulation factor.

38. The pharmaceutical composition of claim 35, wherein the one or more adjuvants are selected from the group consisting of aluminum phosphate, aluminum hydroxide, and phosphated aluminum hydroxide.

39. A vaccine comprising the immunogenic composition of any of claims 1-34 and a pharmaceutically acceptable carrier.

40. A method of making a multivalent vaccine, comprising mixing four or more species of MAPS-X immunogenic complexes of any of claims 1-34 in a single formulation.

41. The method of claim 40, comprising mixing four or more species of MAPS-X immunogenic complexes in a single formulation.

42. Use of immune composition of any of claims 1-34, or the vaccine of claim 39 to induce an immune response to a subject.

43. A method to immunize a subject against a pathogen infection and/or colonization, comprising administering to the subject an immunologically effective amount of a multivalent vaccine comprising at least 2 or more species of MAPS-X immunogenic of any of claims 1-34 to a subject.

44. A method to induce an immune response to a subject, comprising administering to the subject a pharmaceutical composition comprising the immune composition of any of claims 1-34, or the vaccine of claim 39.

45. The method of claim 44, wherein the immune response is an antibody or B cell response.

46. The method of claims 44-45, wherein the immune response is a CD4+ T cell response, including Th1, Th2, or Th17 response, or a CD8+ T cell response, or CD4+/CD8+ T cell response.

47. The method of claim 41, wherein the immune response is:
an antibody or B cell response; and
a T cell response.

48. The method of any of claims 44-47, wherein the immune response is to: at least the first antigenic polysaccharide, or the second antigenic polysaccharides, or both the first and second antigenic polysaccharide, or at least one polypeptide antigen.

49. The method of any of claims 44-48, wherein the immune response is an antibody or B cell response to at least the first antigenic polysaccharide and/or second antigenic polysaccharide, and a CD4+ T cell response, including Th 1, Th2, or Th 17 response, or a CD8+ T cell response, or CD4+/CD8+ T cell response to at least one polypeptide antigen.

50. The method of any of claims 44-49, wherein the immune response is an antibody or B cell response to at least the first antigenic polysaccharide and/or the second antigenic polysaccharide, and an antibody or B cell response and a CD4+ T cell response, including Th1, Th2, or Th 17 response, or a CD8+ T cell response, or CD4+/CD8+ T cell response to at least one polypeptide antigen.

51. The immunogenic composition of any of claims 1-34, wherein herein the immunogenic composition, upon administration to a subject, elicits (i) an immune response to the at least the first antigenic polysaccharide or the second antigenic polysaccharide, or both the first and second antigenic polysaccharide and (ii) an immune response to at least one of the polypeptide antigens, in the subject.

52. The immunogenic composition of claim 51, wherein the immune response to the at least first antigenic polysaccharide or the second antigenic polysaccharide, or both, and/or at least one of the one polypeptide antigens comprises an antibody or B cell response.

53. The immunogenic composition of claim 51, wherein the immune response to the at least first antigenic polysaccharide or the second antigenic polysaccharide, or both, and/or at least one of the one polypeptide antigens comprises a T cell response.

54. The immunogenic composition of claim 51, wherein the immune response to the at least first antigenic polysaccharide or the second antigenic polysaccharide, or both, and/or at least one of the one polypeptide antigens comprises a CD4+ T cell response, including Th1, Th2, or Th17 response, or a CD8+ T cell response, or a CD4+/CD8+ T cell response.

55. The immunogenic composition of claim 51, wherein the immune response to the at least first antigenic polysaccharide or the second antigenic polysaccharide, or both, and/or at least one of the one polypeptide antigens comprises:
an antibody or B cell response; and
a T cell response.

56. The immunogenic composition of any of claims 1-34, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) an antibody or B cell response to at least one of polypeptide antigens, in the subject.

57. The immunogenic composition of any of claims 1-34, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) an antibody or B cell response and a CD4+ T cell response, including Th1, Th2, or Th17 response, or a CD8+ T cell response, or a CD4+/CD8+ T cell response to at least one of polypeptide antigens, in the subject.

58. The immunogenic composition of any of claims 1-34, wherein the immunogenic composition, upon administration to a subject, elicits at least (i) an antibody or B cell response to the at least first antigenic polysaccharide, or second antigenic polysaccharide, or both the first and second antigenic polysaccharide, and (ii) a CD4+ T cell response, including Th1, Th2, or Th17 response, or a CD8+ T cell response, or a CD4+/CD8+ T cell response to at least one of polypeptide antigens, in the subject.

59. The immunogenic composition of any of claims 1-34, wherein a biotin-binding moiety (BBM) comprises an amino acid sequence of at least 80%, or 90%, or 95% sequence identity to SEQ ID NO: 1 that has any one or more of the amino acid modifications: N80, T108, N118, S119A, N138A.

60. The immunogenic composition of any of claims 1-34, wherein a biotin-binding moiety comprises an amino acid sequence of at least 80%, or 90%, or 95%, 98%, 99% or 100% sequence identity to SEQ ID NO: 8, wherein the biotin binding moiety has at least one or more of the amino acid modifications: N80, T108, N118, S119A, N138A.

61. A fusion protein comprising, or consisting of, in any order:
(i) SBD is selected from any of: SBD1, SBD2, SBD3, SBD4, NanH, NanH2, NanH3, VcNanH, or a SBD comprising an amino acid sequence that at least 80%, or at least 85%, or at least 90%, or at least 95% sequence identity to any of: SEQ ID NO: 3-12, and
(ii) a Rhizavidin polypeptide comprising an amino acid sequence having at least 80%, or at least 85%, or at least 90%, or at least 95% sequence identity to SEQ ID NO: 1 or SEQ ID NO: 2.

62. The fusion protein of claim 61, comprising, or consisting essentially of:
a. an amino acid sequence having at least 80%, or at least 85%, or at least 90%, or at least 95% sequence identity to SEQ ID NO: 1 or SEQ ID NO: 2 (Rhavi), and
b. a SBD polypeptide, selected from:
i. an amino acid sequence having at least 80%, or at least 85%, or at least 90%, or at least 95% sequence identity to SEQ ID NO: 3 (SBD1),
ii. an amino acid sequence having at least 80%, or at least 85%, or at least 90%, or at least 95% sequence identity to SEQ ID NO: 4 (SBD2),
iii. an amino acid sequence having at least 80%, or at least 85%, or at least 90%, or at least 95% sequence identity to SEQ ID NO: 5 (SBD3),
iv. an amino acid sequence having at least 80%, or at least 85%, or at least 90%, or at least 95% sequence identity to SEQ ID NO: 6 (SBD4),
v. an amino acid sequence having at least 80%, or at least 85%, or at least 90%, or at least 95% sequence identity to SEQ ID NO: 7 (NanH),
vi. an amino acid sequence having at least 80%, or at least 85%, or at least 90%, or at least 95% sequence identity to SEQ ID NO: 8 (NanH2), or
vii. an amino acid sequence having at least 80%, or at least 85%, or at least 90%, or at least 95% sequence identity to SEQ ID NO: 9 (NanH3).

63. The fusion protein of any of claims 60-62, is selected from any of the fusion proteins comprising an amino acid sequence having at least 80%, or at least 85%, or at least 90%, or at least 95% sequence identity to any of: SEQ ID NO: 9-20.

64. The fusion protein of any of claims 60-62, further comprising at least one linker sequence or at least one spacer sequence located between any of: the BBM and the SBD.

65. The fusion protein of any of claims 60-64, wherein the linker sequence or spacer sequence is selected from any of: GGGGSSS, TDPNSSS, SSS, AAA.

66. The fusion protein of any of claims 60-65, wherein the fusion protein comprises the polypeptide of SEQ ID NO: 13-20, or amino acid sequence having at least 80%, or at least 85%, or at least 90%, or at least 95% sequence identity to any of SEQ ID NOs: 13-20.

67. The fusion protein of any of claims 60-66, wherein the fusion protein consists of the polypeptide of SEQ ID NO: 13-20, or amino acid sequence having at least 80%, or at least 85%, or at least 90%, or at least 95% sequence identity to any of SEQ ID NOs: 13-20.

68. An expression vector comprising a nucleic acid encoding a fusion protein of any of claims 61-67, wherein the expression vector comprises a promoter operatively linked to a nucleic acid sequence encoding the fusion protein, wherein the nucleic acid sequence comprises, in any order,
(i) a nucleic acid sequence encoding a Rhizavidin protein comprising amino acids of SEQ ID NO: 1 or SEQ ID NO: 2 or a protein having at least 80% sequence identity to SEQ ID NO: 1 or SEQ ID NO: 2, and
(ii) a nucleic acid sequence encoding a at least one sialic acid binding domain (SBD) selected from any of: SBD1, SBD2, SBD3, SBD4, NanH, NanH2, NanH3, or VcNanH, or a SBD having an amino acid sequence that has at least 80%, or at least 85%, or at least 90%, or at least 95% sequence identity to any of: SEQ ID NO: 2-12.

69. The expression vector of claim 68, wherein the nucleic encoding a Rhizavidin protein comprises a nucleic acid sequence having at least 80%, or at least 85%, or at least 90%, or at least 95% sequence identity to SEQ ID NO: 78 or a codon optimized variant thereof.

70. The expression vector of claim 68, wherein the nucleic encoding a SBD protein comprises a nucleic acid sequence having at least 80%, or at least 85%, or at least 90%, or at least 95% sequence identity to any of: SEQ ID NO: 80-84, or codon optimized variants thereof.

71. A cell comprising the expression vector of any of claims 68-70.

72. A method of manufacturing a fusion protein of any of claims 61-67, using the expression vector of any of claims 68-70.
